(19) United States
(12) Patent Application Publication
(10) Pub. No.: US 2023/0091682 A1
(43) Pub. Date: Mar. 23, 2023

(54) COGNITIVE DISORDER PREVENTION AND THERAPY

(71) Applicant: ANeuroTech IP BV, Alken (BE)

(72) Inventor: Erik Buntinx, Alken (BE)

(21) Appl. No.: 17/790,408

(22) PCT Filed: Jan. 5, 2021
(86) PCT No.: PCT/EP2021/050091

§ 371 Date: Jun. 30, 2022

(30) Foreign Application Priority Data
Jan. 6, 2020 (EP) PCT/EP2020/050130

(51) Int. Cl. A61K 31/4545; A61P 25/28

(52) U.S. Cl. CPC A61K 31/4545; A61P 25/28

(57) ABSTRACT

The present invention relates to the prevention or treatment of cognitive disorders, in which subjects, in particular subjects at risk of developing a cognitive disorder are treated with dopamine D4 (and serotonin 5-HT2A) receptor antagonists, reverse agonists, or partial agonists.

### FIELD OF THE INVENTION

The invention relates to methods and compositions for preventing or treating cognitive disorders.

### BACKGROUND OF THE INVENTION

Cognitive disorders are a category of mental health disorders that primarily affect cognitive abilities including learning, memory, perception, and problem solving. Cognitive disorders can have numerous causes: genetics, brain trauma, stroke, metabolic and heart issues. The main causes are neurodegenerative diseases such as Alzheimer's disease, Parkinson's disease, and Huntington's disease because they affect or deteriorate brain functions. Other diseases and conditions that cause cognitive disorders include vascular dementia, frontotemporal degeneration, Lewy body disease, prion disease, normal pressure hydrocephalus, and dementia/neurocognitive issues due to HIV infection. They may also include dementia due to substance abuse or exposure to toxins. Cognitive disorder may also be caused by brain trauma, including concussions and Traumatic Brain Injuries, as well as post-traumatic stress and alcoholism. This is referred to as amnesia, and is characterized by damage to major memory encoding parts of the brain such as the hippocampus. Difficulty creating recent term memories is called anterograde amnesia and is caused by damage to the hippocampus part of the brain, which is a major part of the memory process. Retrograde amnesia is also caused by damage to the hippocampus, but the memories that were encoded or in the process of being encoded in long term memory are erased.

Alzheimer's disease is the most common cause of dementia, a general term for memory loss and other cognitive abilities serious enough to interfere with daily life. Alzheimer's disease accounts for 60 percent to 80 percent of dementia cases. Alzheimer's is not a normal part of aging. The greatest known risk factor is increasing age, and the majority of people with Alzheimer's are 65 and older. But Alzheimer's is not just a disease of old age. Other risk factors such as a lifetime history of diabetes mellitus and/or major depressive disorder has been detected as significant contributors to the risk of developing Alzheimer. Approximately 200,000 Americans under the age of 65 have younger-onset Alzheimer's disease (also known as early-onset Alzheimer's). Alzheimer's worsens over time.

Alzheimer's is a progressive disease, where dementia symptoms gradually worsen over a number of years. In its early stages, memory loss is mild, but with late-stage Alzheimer's, individuals lose the ability to carry on a conversation and respond to their environment. Alzheimer's is the sixth leading cause of death in the United States. On average, a person with Alzheimer's lives four to eight years after diagnosis, but can live as long as 20 years, depending on other factors. Alzheimer's has no current cure, but treatments for symptoms are available and research continues. Although current Alzheimer's treatments cannot stop Alzheimer's from progressing, they can temporarily slow the worsening of dementia symptoms and improve quality of life for those with Alzheimer's and their caregivers. Today, there is a worldwide effort under way to find better ways to treat the disease, delay its onset, and prevent it from developing.

The long duration of illness before death contributes significantly to the public health impact of Alzheimer's disease and cognitive disorders in general because much of that time is spent in a state of disability and dependence. Scientists have developed methods to measure and compare the burden of different diseases on a population in a way that takes into account not only the number of people with the condition, but also both the number of years of life lost due to that disease as well as the number of healthy years of life lost by virtue of being in a state of disability. These measures indicate that Alzheimer's is a very burdensome disease, not only to the patients but also to their families and informal caregivers, and that the burden of Alzheimer's has increased more dramatically in the United States than other diseases in recent years. The primary measure of disease burden is called disability-adjusted life years (DALYs), which is the sum of the number of years of life lost due to premature mortality and the number of years lived with disability, totaled across all those with the disease or injury. Using age-standardized DALYs, Alzheimer's rose from the 25th most burdensome disease or injury in the United States in 1990 to the 12th in 2015. In terms of years of life lost, Alzheimer's disease rose from 32nd to 9th, the largest increase for any disease. In terms of years lived with disability, Alzheimer's disease went from ranking 17th to 12th; only kidney disease equalled Alzheimer's in as high a jump in rank. Taken together, these statistics indicate that not only is Alzheimer's disease responsible for the deaths of more and more Americans, but also that the disease is contributing to more and more cases of poor health and disability in the United States.

Accordingly, there is a pressing need not only to develop therapies for Alzheimer's disease and cognitive disorders in general, but equally to develop treatments in order to prevent or delay the development or onset of cognitive disorders, or at least to slow down the progression of these disorders. These and other aspects are addressed by the present invention.

### SUMMARY OF THE INVENTION

The present inventors have surprisingly found that administration of dopamine D4 (and preferably also serotonin 5-HT2A) receptor antagonists, inverse agonists, or partial agonists are suitable for treating cognitive disorders, such as caused by Alzheimer's disease, as well as preventing or delaying the onset or progression of cognitive disorders, and/or for maintaining or improving cognitive function, in particular in subjects at risk of developing a cognitive disorder, and/or for treating an affective disorder, preferably major depressive disorder. The present inventors further have surprisingly found that—administration of dopamine D4 (and preferably also serotonin 5-HT2A) receptor antagonists, inverse agonists, or partial agonists are suitable for reducing suicidal thoughts in subjects, in particular subjects having (a medical history of) an affective disorder, in particular major depressive disorder. In certain embodiments, the subject has an affective disorder or has a medical history of an affective disorder, preferably major depressive disorder, and/or the subject has subjective cognitive decline, mild cognitive impairment, pre-prodromal Alzheimer's disease, prodromal Alzheimer's disease, or preclinical Alzheimer's disease. In certain preferred embodiments, the subject has an affective disorder or has a medical history of an affective disorder, preferably major depressive disorder, and further has subjective cognitive decline, mild cognitive impairment, pre-prodromal Alzheimer's disease, prodromal Alzheimer's disease, or preclinical Alzheimer's disease. In certain embodiments, the subject having (a medical history of) an affective disorder is under treatment with anti-affective disorder medication, such as an antidepressant.

In particular, the inventors have found that that administration of dopamine D4 (and serotonin 5-HT2A) receptor antagonists, inverse agonists, or partial agonists can prevent or delay, and even reverse cognitive impairment, and/or maintain or improve cognitive function, in particular in subjects at risk of developing a cognitive disorder. In certain embodiments, the subject at risk of developing a cognitive disorder has an affective disorder or has a medical history of an affective disorder, preferably major depressive disorder, and/or the subject has subjective cognitive decline, mild cognitive impairment, pre-prodromal Alzheimer's disease, prodromal Alzheimer's disease, or preclinical Alzheimer's disease. In certain preferred embodiments, the subject at risk of developing a cognitive disorder has an affective disorder or has a medical history of an affective disorder, preferably major depressive disorder, and further has subjective cognitive decline, mild cognitive impairment, pre-prodromal Alzheimer's disease, prodromal Alzheimer's disease, or preclinical Alzheimer's disease.

In certain preferred embodiments, the subjects to be treated according to the invention are subjects having an increased risk of developing a cognitive disorder, such as caused by Alzheimer's disease.

In a preferred embodiment, the subject to be treated according to the invention, preferably the subject having an increased risk of developing a cognitive disorder, is characterized by having one or more of, preferably all, (i) (a medical history of) an affective disorder, such as major depressive disorder, (ii) a mini mental state examination (MMSE) score ranging from 25 to 29 or 25 to 30, preferably 26 to 29 or 26 to 30, more preferably 27 to 29 or 27 to 30, (iii) a sleep disorder, such as insomnia, preferably acute insomnia, and/or at least one clinical relevant restlessness event at night or during sleep, and/or (iv) a wake after sleep onset (WASO) of at least 30 minutes, preferably at least 40 minutes, more preferably at least 50 minutes.

The present inventors have further surprisingly found that administration of dopamine D4 (and serotonin 5-HT2A) receptor antagonists, inverse agonists, or partial agonists and application of transcranial direct current stimulation act synergistically in treating cognitive disorders, such as caused by Alzheimer's disease, as well as preventing or delaying the onset or progression of cognitive disorders, and/or for maintaining or improving cognitive function.

In particular, the inventors have found that that concomitant administration of dopamine D4 (and serotonin 5-HT2A) receptor antagonists, inverse agonists, or partial agonists and application of transcranial direct current stimulation can prevent or delay, and even reverse cognitive impairment, and/or for maintain or improve cognitive function.

Furthermore, the inventors have found that administration of dopamine D4 (and serotonin 5-HT2A) receptor antagonists, inverse agonists, or partial agonists consolidates the beneficial effects of transcranial direct current stimulation, such that the beneficial effects of transcranial direct current stimulation on the development or evolution of cognitive impairment are prolonged and even maintained over time.

In this context, the inventors have found that not only the combined administration of dopamine D4 (and serotonin 5-HT2A) receptor antagonists, inverse agonists, or partial agonists and application of transcranial direct current stimulation has an increased effect in improving cognitive impairment, but also that after initial combined treatment, the beneficial effects are maintained by continued administration of the dopamine D4 (and serotonin 5-HT2A) receptor antagonists, inverse agonists, or partial agonists without concomitant application of transcranial direct current stimulation.

The present invention is in particular captured by the appended claims, which are hereby incorporated explicitly by reference.

In an aspect, the invention relates to a D4 (and 5-HT2A) receptor antagonist, reverse agonist, or partial agonist for use in preventing, delaying the onset or progression, and/or treating of a cognitive disorder in a subject, and/or for use in maintaining or improving cognitive function.

In an aspect, the invention relates to a D4 (and 5-HT2A) receptor antagonist, reverse agonist, or partial agonist for use in reducing suicidal thoughts or preventing suicide of a subject.

In certain embodiments, the invention relates to a D4 (and 5-HT2A) receptor antagonist, reverse agonist, or partial agonist for use in preventing, delaying the onset or progression, and/or treating of a cognitive disorder, and/or for use in maintaining or improving cognitive function in a subject which is, will be, or has been treated with transcranial direct current stimulation (tDCS).

In certain embodiments, the invention relates to a D4 (and 5-HT2A) receptor antagonist, reverse agonist, or partial agonist for use in preventing, delaying the onset or progression, and/or treating of a cognitive disorder in a subject which is characterised as having been treated previously with a D4 (and 5-HT2A) receptor antagonist, reverse agonist, or partial agonist and transcranial direct current stimulation (tDCS).

In certain preferred embodiments, the D4 (and 5-HT2A) receptor antagonist, reverse agonist, or partial agonist is pipamperone.

In certain preferred embodiments, tDCS is applied to the prefrontal cortex, such as the dorsolateral prefrontal cortex (DLPFC), preferably anodal tDCS applied to the left DLPFC. Preferably the anode is applied to the left DLPFC and the cathode is applied to the right DLPFC.

### BRIEF DESCRIPTION OF THE FIGURES

FIG. 1: Evolution of the MMSE score and number of qualified clinical restlessness events at night. Left bar: MMSE and number of qualified clinical restlessness events at night before treatment. Right bar: MMSE and number of qualified clinical restlessness events at night after three weeks of treatment with 20 mg daily pipamperone and 15 sessions (5 sessions per week) of anodal tDCS (2.0 mA) applied to the left dorsolateral prefrontal cortex (30 minutes per session).

FIG. 2: (A) Evolution of the relative gamma power at the frontal cortex during total REM sleep in subjects at risk of developing a cognitive disorder such as caused by Alzheimer's disease (PT 1 and PT3) compared to a subject having developed Alzheimer's disease (PT2), after 31 days of treatment with 20 mg pipamperone daily. (B) Evolution of the MMSE in subjects at risk of developing a cognitive disorder such as caused by Alzheimer's disease (PT 1 and PT3) compared to a subject having developed Alzheimer's disease (PT2), after 31 days of treatment with 20 mg pipamperone daily. (C) Percentage change of the relative gamma power at the frontal cortex during total REM sleep and MMSE in subjects at risk of developing a cognitive disorder such as caused by Alzheimer's disease (PT 1 and PT3) compared to a subject having developed Alzheimer's disease (PT2), after 31 days of treatment with 20 mg pipamperone daily.

FIG. 3: The international 10-20 system of electrode placement.

FIG. 4: Statistically significant superior effect (change from baseline score) of pipamperone compared to citalopram on cognitive enhancement (A) and reduction of suicidal thoughts (B). Cognitive enhancement and suicidal thoughts were evaluated respectively according to the Montgomery-Asberg Depression Rating Scale (MADRS) item 6 and item 10, after 10 weeks of study treatment as assessed by the clinical rater.

FIG. 5: Proportion of patients exhibiting a clinically relevant improvement of cognitive impairment (reduced ability to read/communicate) after administration of pipamperone (PIP), citalopram (CIT), and a combination of pipamperone and citalopram (PIP+CIT). Remission rate of reduced ability to read/communicate is defined as a MADRS Item 6 total score at Baseline (Day 1)>3 to (end of) Week 10 at Week 10 as assessed by the clinical rater.

FIG. 6: Change from baseline in total MADRS score after administration of pipamperone (PIP), citalopram (CIT), and a combination of pipamperone and citalopram (PIP+CIT) after the indicated periods, baseline (at the start of treatment), week 2, week 3, week 4, week 6, and week 10. Change from baseline in total score on MADRS after the indicated period of study treatment as assessed by the clinical rater.

FIG. 7: Proportion of patients exhibiting major depressive disorder (MDD) in remission after administration of pipamperone (PIP), citalopram (CIT), and a combination of pipamperone and citalopram (PIP+CIT) after the indicated periods, baseline (at the start of treatment), week 2, week 3, week 4, week 6, and week 10. Remission is defined as a MADRS total score ≤10 at the indicated period as assessed by the clinical rater.

### DETAILED DESCRIPTION OF THE INVENTION

Before the present system and method of the invention are described, it is to be understood that this invention is not limited to particular systems and methods or combinations described, since such systems and methods and combinations may, of course, vary. It is also to be understood that the terminology used herein is not intended to be limiting, since the scope of the present invention will be limited only by the appended claims.

As used herein, the singular forms “a”, “an”, and “the” include both singular and plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. It will be appreciated that the terms “comprising”, “comprises” and “comprised of” as used herein comprise the terms “consisting of”, “consists” and “consists of”, as well as the terms “consisting essentially of”, “consists essentially” and “consists essentially of”.

The recitation of numerical ranges by endpoints includes all numbers and fractions subsumed within the respective ranges, as well as the recited endpoints.

The term “about” or “approximately” as used herein when referring to a measurable value such as a parameter, an amount, a temporal duration, and the like, is meant to encompass variations of +/−20% or less, preferably +/−10% or less, more preferably +/−5% or less, and still more preferably +/−1% or less of and from the specified value, insofar such variations are appropriate to perform in the disclosed invention. It is to be understood that the value to which the modifier “about” or “approximately” refers is itself also specifically, and preferably, disclosed.

Whereas the terms “one or more” or “at least one”, such as one or more or at least one member(s) of a group of members, is clear per se, by means of further exemplification, the term encompasses inter alia a reference to any one of said members, or to any two or more of said members, such as, e.g., any ≥3, ≥4, ≥5, ≥6, or ≥7 etc. of said members, and up to all said members.

All references cited in the present specification are hereby incorporated by reference in their entirety. In particular, the teachings of all references herein specifically referred to are incorporated by reference.

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the present invention.

In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention, and form different embodiments, as would be understood by those in the art. For example, in the appended claims, any of the claimed embodiments can be used in any combination.

In the following detailed description of the invention, reference is made to the accompanying drawings that form a part hereof, and in which are shown by way of illustration only of specific embodiments in which the invention may be practiced. It is to be understood that other embodiments may be utilised and structural or logical changes may be made without departing from the scope of the present invention. The following detailed description, therefore, is not to be taken in a limiting sense, and the scope of the present invention is defined by the appended claims.

Preferred statements (features) and embodiments of this invention are set herein below. Each statements and embodiments of the invention so defined may be combined with any other statement and/or embodiments unless clearly indicated to the contrary. In particular, any feature indicated as being preferred or advantageous may be combined with any other feature or features or statements indicated as being preferred or advantageous. Hereto, the present invention is in particular captured by any one or any combination of one or more of the below numbered aspects and embodiments 1 to 65, with any other statement and/or embodiments.
1. A D4 (and preferably also 5-HT2A) receptor antagonist, reverse agonist, or partial agonist for use in preventing, delaying or postponing the onset or delaying or postponing progression, and/or treating of a cognitive disorder (such as subjective cognitive decline) or an affective disorder, preferably (mild) major depressive disorder, in a subject, and/or for use in maintaining or improving cognitive function in a subject, and/or for use in maintaining or increasing gamma power, preferably relative gamma power, preferably at the cortex, preferably at the frontal cortex, such as the (dorsolateral) pre-frontal cortex, preferably during sleep, preferably REM sleep, and/or for use in reducing suicidal thoughts in a subject or for preventing suicide of a subject, optionally wherein said subject has (a medical history of) an affective disorder, preferably (mild) major depressive disorder which is (to be) treated with an antidepressant.
2. The D4 (and 5-HT2A) receptor antagonist, reverse agonist, or partial agonist for use according to statement 1, wherein said subject is, will be, or has been treated with transcranial direct current stimulation (tDCS).
3. The D4 (and 5-HT2A) receptor antagonist, reverse agonist, or partial agonist for use according to statement 2, wherein said subject was previously treated with a D4 (and 5-HT2A) receptor antagonist, reverse agonist, or partial agonist and tDCS.
4. The D4 (and 5-HT2A) receptor antagonist, reverse agonist, or partial agonist for use according to statement 2 or 3,
   wherein said subject was previously treated with said D4 (and 5-HT2A) receptor antagonist, reverse agonist, or partial agonist and tDCS and exhibited an MMSE score increase of at least 1 point, preferably at least 2 points, and/or a decrease in qualified clinical restlessness events during sleep of at least 25%, preferably at least 50%.
5. A D4 (and 5-HT2A) receptor antagonist, reverse agonist, or partial agonist as an adjunctive therapy to tDCS for use in preventing, delaying or postponing the onset or delaying or postponing progression, and/or treating of a cognitive disorder in a subject, and/or for maintaining or improving cognitive function in a subject, and/or for use in maintaining or increasing gamma power, preferably relative gamma power, preferably at the cortex, preferably at the frontal cortex, such as the (dorsolateral) pre-frontal cortex, preferably during sleep, preferably REM sleep.
6. A D4 (and 5-HT2A) receptor antagonist, reverse agonist, or partial agonist for use as an adjunctive therapy to tDCS to prevent, delay or postpone the onset or delay or postpone progression, and/or treat a cognitive disorder in a subject, and/or for maintaining or improving cognitive function in a subject, and/or for use in maintaining or increasing gamma power, preferably relative gamma power, preferably at the cortex, preferably at the frontal cortex, such as the (dorsolateral) pre-frontal cortex, preferably during sleep, preferably REM sleep.
7. A D4 (and 5-HT2A) receptor antagonist, reverse agonist, or partial agonist for use in increasing the MMSE score and/or decreasing the number of qualified clinical restlessness events during sleep in a subject, and/or for use in maintaining or increasing gamma power, preferably relative gamma power, preferably at the cortex, preferably at the frontal cortex, such as the (dorsolateral) pre-frontal cortex, preferably during sleep, preferably REM sleep.
8. The D4 (and 5-HT2A) receptor antagonist, reverse agonist, or partial agonist for use according to statement 7, wherein said subject is, will be, or has been treated with tDCS.
9. A D4 (and 5-HT2A) receptor antagonist, reverse agonist, or partial agonist as an adjunctive therapy to tDCS for use in increasing the MMSE score and/or decreasing the number of qualified clinical restlessness events during sleep in a subject, and/or for use in maintaining or increasing gamma power, preferably relative gamma power, preferably at the cortex, preferably at the frontal cortex, such as the (dorsolateral) pre-frontal cortex, preferably during sleep, preferably REM sleep.
10. A D4 (and 5-HT2A) receptor antagonist, reverse agonist, or partial agonist for use as an adjunctive therapy to tDCS to increase the MMSE score and/or decrease the number of qualified clinical restlessness events during sleep in a subject, and/or for use in maintaining or increasing gamma power, preferably relative gamma power, preferably at the cortex, preferably at the frontal cortex, such as the (dorsolateral) pre-frontal cortex, preferably during sleep, preferably REM sleep.
11. The D4 (and 5-HT2A) receptor antagonist, reverse agonist, or partial agonist for use according to any of statements 7 to 10, wherein said MMSE score is increased with at least 1 point, preferably at least 2 points, and/or wherein the qualified clinical restlessness events during sleep are reduced with at least 25%, preferably at least 50%.
12. The D4 (and 5-HT2A) receptor antagonist, reverse agonist, or partial agonist for use according to any of statements 1 to 11 which is pipamperone.
13. The D4 (and 5-HT2A) receptor antagonist, reverse agonist, or partial agonist for use according to any of statements 1 to 12, wherein said cognitive disorder is dementia or is caused by dementia, (dementia) caused by Alzheimer's Disease, substance-related persisting dementia, vascular dementia, dementia due to HIV disease, dementia with Lewy bodies, dementia due to head trauma/chronic traumatic encephalopathy, dementia due to Parkinson Disease, dementia due to Huntington Disease, dementia due to Pick Disease (frontotemporal dementia), dementia due to prions (e.g. Creutzfedt-Jacob Disease), Normal Pressure Hydrocephalus, subdural hematoma, posterior cortical atrophy, corticobasal degeneration, progressive supranuclear palsy, mixed dementia, medication side effects, chronic alcoholism, tumors or infections in the brain, toxins (e.g. lead), Wernicke-Korsakoff, hypothyroidism, vitamin B12 deficiency, Lyme disease, and neurosyphillis, Behcet's disease, multiple sclerosis, sarcoidosis, Sjögren's syndrome, systemic lupus erythematosus, celiac disease, and non-celiac gluten sensitivity, vitamin B12 deficiency, folate deficiency, niacin deficiency, and infective causes including cryptococcal meningitis, AIDS, Lyme disease, progressive multifocal leukoencephalopathy, subacute sclerosing panencephalitis, syphilis, and Whipple's disease, Alexander disease, Canavan disease, Cerebrotendinous xanthomatosis, Dentatorubral-pallidoluysian atrophy, Epilepsy, Fatal familial insomnia, Fragile X-associated tremor/ataxia syndrome, Glutaric aciduria type 1, Krabbe's disease, Maple syrup urine disease, Niemann-Pick disease type C, Neuronal ceroid lipofuscinosis, Neuroacanthocytosis, Organic acidemias, Pelizaeus-Merzbacher disease, Sanfilippo syndrome type B, Spinocerebellar ataxia type 2, Urea cycle disorders, amnestic disorders due to a general medical condition, substance-induced persisting amnestic disorder, mild cognitive impairment disorder.
14. The D4 (and 5-HT2A) receptor antagonist, reverse agonist, or partial agonist for use according to any of statements 1 to 13, wherein said cognitive disorder is Alzheimer's Disease or dementia caused by Alzheimer's Disease or Alzheimer's Disease-caused dementia.
15. The D4 (and 5-HT2A) receptor antagonist, reverse agonist, or partial agonist for use according to any of statements 1 to 14, wherein said subject has an increased risk of developing said cognitive disorder.
16. The D4 (and 5-HT2A) receptor antagonist, reverse agonist, or partial agonist for use according to statement 15, wherein said increased risk of developing said cognitive disorder is selected from (medical history of) major depression, diabetes mellitus, renal function impairment, hypertension, hypercholesterolemia, hyperlipidemia, cardio-vascular disease (including coronary artery disease, atrial fibrillations, heart failure, or valvular heart disease), cerebro-vascular disease, gastrointestinal disorder, anemia, inflammation, oxidative stress, chronic obstructive pulmonary disease, liver function impairment (including cirrhosis), sleep disorder or any medical condition which is related to an increased incidence of developing a cognitive disorder, but also genetic predisposition, age above 60, preferably above 65.
17. The D4 (and 5-HT2A) receptor antagonist, reverse agonist, or partial agonist for use according to any of statements 1 to 16, wherein said subject has (a medical history of) an affective disorder.
18. The D4 (and 5-HT2A) receptor antagonist, reverse agonist, or partial agonist for use according to statement 17, wherein said affective disorder is (mild) major depressive disorder, bipolar disorder, or anxiety disorder, preferably (mild) major depressive disorder.
19. The D4 (and 5-HT2A) receptor antagonist, reverse agonist, or partial agonist for use according to any of statements 1 to 18, wherein said subject has an MMSE score ranging from 27 to 29, 26 to 29, or 25 to 29, or ranging from 27 to 30, 26 to 30, or 25 to 30.
20. The D4 (and 5-HT2A) receptor antagonist, reverse agonist, or partial agonist for use according to any of statements 1 to 19, wherein said subject exhibits sleep structure deterioration.
21. The D4 (and 5-HT2A) receptor antagonist, reverse agonist, or partial agonist for use according to any of statements 1 to 20, wherein said subject has or exhibits a sleep disorder, preferably (acute) insomnia.
22. The D4 (and 5-HT2A) receptor antagonist, reverse agonist, or partial agonist for use according to any of statements 1 to 21, wherein said subject exhibits at least one clinical relevant restlessness event during sleep (preferably during wake after sleep onset), preferably at least two, more preferably at least three.
23. The D4 (and 5-HT2A) receptor antagonist, reverse agonist, or partial agonist for use according to statement 22, wherein said clinical relevant restlessness events are determined by EEG and EMG.
24. The D4 (and 5-HT2A) receptor antagonist, reverse agonist, or partial agonist for use according to statement 23, wherein said clinical relevant restlessness events are determined by polysomnography (PSG).
25. The D4 (and 5-HT2A) receptor antagonist, reverse agonist, or partial agonist for use according to statement 17, wherein said clinical relevant restlessness events are determined by video imaging, preferably infrared video imaging and/or wherein said clinical restlessness event is or includes a visible movement, preferably agitation.
26. The D4 (and 5-HT2A) receptor antagonist, reverse agonist, or partial agonist for use according to statement 22, wherein said clinical relevant restlessness events are characterised by noise-making, preferably at least 50 dB.
27. The D4 (and 5-HT2A) receptor antagonist, reverse agonist, or partial agonist for use according to any of statements 22 to 26, wherein said clinical relevant restlessness events are or include movement artefacts.
28. The D4 (and 5-HT2A) receptor antagonist, reverse agonist, or partial agonist for use according to any of statements 22 to 27, wherein said clinical relevant restlessness events do not include (sleep) apnea.
29. The D4 (and 5-HT2A) receptor antagonist, reverse agonist, or partial agonist for use according to any of statements 1 to 28, wherein said subject has a wakefulness after sleep onset (WASO) score of at least 30 minutes, preferably at least 40 minutes, more preferably at least 50 minutes.
30. The D4 (and 5-HT2A) receptor antagonist, reverse agonist, or partial agonist for use according to any of statements 12 to 29, wherein said pipamperone is to be administered at a daily dose ranging from 5 to 20 mg or 4 to 20 mg, preferably 5 to 20 mg.
31. The D4 (and 5-HT2A) receptor antagonist, reverse agonist, or partial agonist for use according to any of statements 1 to 30, wherein said The D4 (and 5-HT2A) receptor antagonist, reverse agonist, or partial agonist is to be administered within 24 hours before or after tDCS.
32. The D4 (and 5-HT2A) receptor antagonist, reverse agonist, or partial agonist for use according to any of statements 1 to 31, wherein said subject is, will be, or has been treated with tDCS for at least one week, preferably at least two weeks, more preferably at least three weeks.
33. The D4 (and 5-HT2A) receptor antagonist, reverse agonist, or partial agonist for use according to any of statements 1 to 32, wherein said subject is, will be, or has been treated with tDCS for at most 12 weeks, preferably at most 10 weeks, more preferably at most 8 weeks.
34. The D4 (and 5-HT2A) receptor antagonist, reverse agonist, or partial agonist for use according to any of statements 1 to 33, wherein said subject is, will be, or has been treated daily with tDCS.
35. The D4 (and 5-HT2A) receptor antagonist, reverse agonist, or partial agonist for use according to any of statements 1 to 34, wherein said subject is, will be, or has been treated at least 5 times per week with tDCS
36. The D4 (and 5-HT2A) receptor antagonist, reverse agonist, or partial agonist for use according to any of statements 1 to 35, wherein said subject is treated with said D4 (and 5-HT2A) receptor antagonist, reverse agonist, or partial agonist and tDCS followed by treatment with said D4 (and 5-HT2A) receptor antagonist, reverse agonist, or partial agonist without concomitant tDCS.
37. The D4 (and 5-HT2A) receptor antagonist, reverse agonist, or partial agonist for use according to any of statements 1 to 36, wherein said subject is treated with said D4 (and 5-HT2A) receptor antagonist, reverse agonist, or partial agonist and tDCS for a period ranging from one to twelve weeks followed by treatment with said D4 (and 5-HT2A) receptor antagonist, reverse agonist, or partial agonist without concomitant tDCS.
38. The D4 (and 5-HT2A) receptor antagonist, reverse agonist, or partial agonist for use according to any of statements 1 to 37, wherein said subject has:
   (i) an MMSE score ranging from 25 to 29, preferably 26 to 29, more preferably 27 to 29, or an MMSE score ranging from 25 to 30, preferably 26 to 30, more preferably 27 to 30;
   (ii) a sleep disorder, preferably (acute) insomnia, and/or exhibits at least one clinical relevant restlessness event during sleep;
   (iii) (a medical history of) an affective disorder, preferably (mild) major depressive disorder; and
   (iv) a wake after sleep onset (WASO) score of at least 30 minutes, preferably at least 40 minutes, more preferably at least 50 minutes.
39. The D4 (and 5-HT2A) receptor antagonist, reverse agonist, or partial agonist for use according to any of statements 1 to 38, wherein the (relative) gamma power is increased with at least 1%, preferably at least 2%, more preferably at least 5%, such as at least 10%.
40. The D4 (and 5-HT2A) receptor antagonist, reverse agonist, or partial agonist for use according to any of statements 1 to 39, wherein said subject has subjective cognitive decline (SCD).
41. The D4 (and 5-HT2A) receptor antagonist, reverse agonist, or partial agonist for use according to any of statements 1 to 40, wherein said subject has a pre-prodromal neurodegenerative disease, preferably pre-prodromal Alzheimer's disease.
42. The D4 (and 5-HT2A) receptor antagonist, reverse agonist, or partial agonist for use according to any of statements 1 to 40, wherein said subject has a prodromal neurodegenerative disease, preferably prodromal Alzheimer's disease.
43. The D4 (and 5-HT2A) receptor antagonist, reverse agonist, or partial agonist for use according to any of statements 1 to 42, wherein said subject has (pre-)mild cognitive impairment (MCI).
44. The D4 (and 5-HT2A) receptor antagonist, reverse agonist, or partial agonist for use according to any of statements 1 to 43, wherein said subject has a pre-clinical or asymptomatic neurodegenerative disease, preferably pre-clinical or asymptomatic Alzheimer's disease.
45. The D4 (and 5-HT2A) receptor antagonist, reverse agonist, or partial agonist for use according to any of statements 1 to 44, wherein said subject has not been diagnosed with a neurodegenerative disease, preferably Alzheimer's disease.
46. The D4 (and 5-HT2A) receptor antagonist, reverse agonist, or partial agonist for use according to any of statements 1 to 45, wherein said subject has an increased risk of developing a (clinical) neurodegenerative disease, preferably (clinical) Alzheimer's disease.
47. The D4 (and 5-HT2A) receptor antagonist, reverse agonist, or partial agonist for use according to any of statements 1 to 46, wherein said subject has an increased risk of developing a neurocognitive disorder, preferably major neurocognitive disorder, preferably caused by Alzheimer's disease.
48. The D4 (and 5-HT2A) receptor antagonist, reverse agonist, or partial agonist for use according to any of statements 1 to 47, wherein said subject has an affective disorder, preferably (mild) major depressive disorder.
49. The D4 (and 5-HT2A) receptor antagonist, reverse agonist, or partial agonist for use according to any of statements 1 to 48, wherein said subject is at least 50 years old, preferably at least 60 years old, more preferably at least 65 years old, such as at least 70 years old.
50. The D4 (and 5-HT2A) receptor antagonist, reverse agonist, or partial agonist for use according to any of statements 1 to 49, wherein said subject has (mild) major depressive disorder in (full or partial) remission.
51. The D4 (and 5-HT2A) receptor antagonist, reverse agonist, or partial agonist for use according to any of statements 1 to 50, wherein said subject is (to be) treated with an antidepressant.
52. The D4 (and 5-HT2A) receptor antagonist, reverse agonist, or partial agonist for use according to any of statements 1 to 51, wherein said subject is (to be) treated with a medicament selected from selective serotonin reuptake inhibitors (SSRI) (such as citalopram (e.g. Celexa), escitalopram (e.g. Lexapro), fluoxetine (e.g. Prozac), fluvoxamine (e.g. Luvox), paroxetine (e.g. Paxil), sertraline (e.g. Zoloft), dapoxetine (e.g. Prilligy), indalpine (e.g. Upstène), zimelidine (e.g. Zelmid), alaproclate (GEA-654), centpropazine, cericlamine (JO-1017), femoxetine (Malexil; FG-4963), ifoxetine (CGP-15210), omiloxetine, panuramine (WY-26002), pirandamine (AY-23713), seproxetine ((S)-norfluoxetine)), serotonin-norepinephrine reuptake inhibitors (SNRI) (such as atomoxetine (e.g. Strattera), desvenlafaxine (e.g. Pristiq, Khedezla), duloxetine (e.g. Cymbalta, Irenka), levomilnacipran (e.g. Fetzima), milnacipran (e.g. Ixel, Savella, Impulsor), sibutramine (e.g. Meridia), tramadol (e.g. Ultram), venlafaxine (e.g. Effexor)), serotonin modulators and stimulators (SMS) (such as vortioxetine, vilazodone), serotonin antagonists and reuptake inhibitors (SARI) (such as etoperidone (e.g. Axiomin, Etonin), lorpiprazole (e.g. Normarex), mepiprazole (e.g. Psigodal), nefazodone (e.g. Serzone, Nefadar), trazodone (e.g. Desyrel), vilazodone (e.g. Viibryd), vortioxetine (e.g. Trintellix), niaprazine (e.g. Nopron), medifoxamine (e.g.Cledial, Gerdaxyl), lubazodone), norepinephrine reuptake inhibitors (NRI or NERI) (such as amedalin (UK-3540-1), atomoxetine (e.g. Strattera), CP-39,332, daledalin (UK-3557-15), edivoxetine (LY-2216684), esreboxetine, lortalamine (LM-1404), nisoxetine (LY-94,939), reboxetine (e.g. Edronax, Vestra), talopram (e.g. tasulopram) (Lu 3-010), talsupram (Lu 5-005), tandamine (AY-23,946), viloxazine (Vivalan), including NRIs with activity at other sites such as bupropion (e.g. Wellbutrin, Zyban), ciclazindol (Wy-23,409), duloxetine, manifaxine (GW-320,659), maprotiline (e.g. Deprilept, Ludiomil, Psymion), radafaxine (GW-353,162), tapentadol (e.g. Nucynta), teniloxazine (e.g. Lucelan, Metatone), protriptyline (e.g. Vivactil), nortriptyline (e.g. Pamelor), desipramine (e.g. Norpramin)), norepinephrine-dopamine reuptake inhibitors (NDRI) (such as bupropion), tricyclic antidepressants (TCA) (such as butriptyline (e.g. Evadyne), clomipramine (e.g. Anafranil), imipramine (e.g. Tofranil, Janimine, Praminil), trimipramine (e.g. Surmontil), desipramine (e.g. Norpramin, Pertofrane), dibenzepin (e.g. Noveril, Victoril), lofepramine (e.g. Lomont, Gamanil), maprotiline (e.g. Ludiomil), nortriptyline (e.g. Pamelor, Aventyl, Norpress), protriptyline (e.g.Vivactil), amitriptyline (e.g.Elavil, Endep), amitriptylinoxide (e.g.Amioxid, Ambivalon, Equilibrin), amoxapine (e.g. Asendin), demexiptiline (e.g. Deparon, Tinoran), dimetacrine (e.g. Istonil, Istonyl, Miroistonil), dosulepin (e.g. Prothiaden), doxepin (e.g. Adapin, Sinequan), fluacizine (e.g. Phtorazisin), imipraminoxide (e.g. Imiprex, Elepsin), melitracen (e.g. Deanxit, Dixeran, Melixeran, Trausabun), metapramine (e.g. Timaxel), nitroxazepine (e.g. Sintamil), noxiptiline (e.g. Agedal, Elronon, Nogedal), pipofezine (e.g. Azafen/Azaphen), propizepine (e.g. Depressin, Vagran), quinupramine (e.g. Kevopril, Kinupril, Adeprim, Quinuprine), amineptine (e.g. Survector, Maneon, Directim), iprindole (e.g. Prondol, Galatur, Tetran), opipramol (e.g. Insidon, Pramolan, Ensidon, Oprimol), tianeptine), tetracyclic antidepressants (TeCA) (such as maprotiline (e.g. Ludiomil), mianserin (e.g. Tolvon), mirtazapine (e.g. Remeron), setiptiline (e.g. Tecipul), amoxapine (e.g. Asendin), benzoctamine (e.g. Tacitin), loxapine (e.g. Adasuve, Loxitane), mazindol (e.g. Mazanor, Sanorex, aptazapine (CGS-7525A), esmirtazapine (ORG-50,081), oxaprotiline (C 49-802 BDA), ciclazindol (WY-23,409)), monoamine oxidase inhibitors (MAOI) (such as isocarboxazid (e.g. Marplan), nialamide (e.g. Niamid), phenelzine (e.g. Nardil, Nardelzine), hydracarbazine, tranylcypromine (e.g. Parnate, Jatrosom), bifemelane (e.g. Alnert, Celeport), moclobemide (e.g. Aurorix, Manerix), pirlindole (e.g. Pirazidol, toloxatone (e.g. Humoryl), rasagiline (e.g. Azilect), selegiline (e.g. Deprenyl, Eldepryl, Emsam, Zelapar), safinamide (e.g. Xadago), linezolid, benmoxin (e.g. Nerusil, Neuralex), iproclozide (e.g. Sursum), iproniazid (e.g. Marsilid, Iprozid, Ipronid, Rivivol, Propilniazida), mebanazine (e.g. Actomol), octamoxin (e.g. Ximaol, Nimaol), pheniprazine (e.g. Catron), phenoxypropazine (e.g. Drazine), pivalylbenzhydrazine (e.g. Tersavid), safrazine (e.g. Safra), caroxazone (e.g. Surodil, Timostenil), minaprine (e.g. Cantor), brofaromine (e.g. Consonar), caroxazone (e.g. Surodil, Timostenil), eprobemide (e.g. Befol), methylene blue, metralindole (e.g. Inkazan), minaprine (e.g. Cantor), moclobemide (e.g. Aurorix, Manerix), pirlindole (e.g. Pirazidol), toloxatone (e.g. Humoryl), curcumin, harmaline, harmine, amiflamine (FLA-336), befloxatone (MD-370,503), cimoxatone (MD-780,515), esuprone, sercloremine (CGP-4718-A), tetrindole, CX157 (TriRima)), and NMDA receptor antagonists (e.g. ketamine, esketamine), preferably said medicament is a selective serotonin reuptake inhibitors (SSRI), preferably citalopram or escitalopram.
53. The D4 (and 5-HT2A) receptor antagonist, reverse agonist, or partial agonist for use according to any of statements 1 to 52, wherein said subject does not have psychotic features, preferably delusions, hallucinations, disorganized speech, and/or grossly disorganized or catatonic behavior (as for instance assessed by the Mini International Neuropsychiatric Interview (MINI)).
54. The D4 (and 5-HT2A) receptor antagonist, reverse agonist, or partial agonist for use according to any of statements 1 to 53, wherein said subject does not have psychosis.
55. The D4 (and 5-HT2A) receptor antagonist, reverse agonist, or partial agonist for use according to any of statements 1 to 54, wherein said subject has (mild) mild depressive symptoms.
56. The D4 (and 5-HT2A) receptor antagonist, reverse agonist, or partial agonist for use according to any of statements 1 to 55, wherein said D4 (and preferably also 5-HT2A) receptor antagonist, reverse agonist, or partial agonist, preferably pipamperone, is (to be) administered ad noctum.
57. The D4 (and 5-HT2A) receptor antagonist, reverse agonist, or partial agonist according to any of statements 1 to 56, for use of treating an affective disorder, preferably (mild) major depressive disorder.
58. The D4 (and 5-HT2A) receptor antagonist, reverse agonist, or partial agonist for use according to statement 57, wherein the (total) MADRS score is reduced by at least 40%, preferably at least 45%, more preferably at least 50% after 10 weeks.
59. The D4 (and 5-HT2A) receptor antagonist, reverse agonist, or partial agonist for use according to statement 57 or 58, wherein the (total) MADRS score is reduced by at least 40%, preferably at least 45%, more preferably at least 50% after 6 weeks.
60. The D4 (and 5-HT2A) receptor antagonist, reverse agonist, or partial agonist for use according to any of statements 57 to 59, wherein at least 35%, preferably at least 40% of subjects achieve remission after 10 weeks, preferably as determined by a (total) MADRS score of at most 10.
61. The D4 (and 5-HT2A) receptor antagonist, reverse agonist, or partial agonist for use according to any of statements 57 to 60, wherein at least 32% of subjects achieve remission after 6 weeks, preferably as determined by a (total) MADRS score of at most 10.
62. The D4 (and 5-HT2A) receptor antagonist, reverse agonist, or partial agonist for use according to any of statements 57 to 61, wherein cognitive function (e.g. concentration difficulties) is improved by at least 20%, preferably at least 30%, preferably as determined according to the MADRS item 6.
63. The D4 (and 5-HT2A) receptor antagonist, reverse agonist, or partial agonist for use according to any of statements 57 to 61, wherein cognitive function (e.g. concentration difficulties) is improved by at least 1 point, preferably at least 2 points according to the MADRS item 6.
64. The D4 (and 5-HT2A) receptor antagonist, reverse agonist, or partial agonist for use according to any of statements 57 to 61, wherein suicidal thoughts are reduced by at least 20%, preferably at least 30%, preferably as determined according to the MADRS item 6.
65. The D4 (and 5-HT2A) receptor antagonist, reverse agonist, or partial agonist for use according to any of statements 57 to 61, wherein suicidal thoughts are reduced by at least 1 point, preferably at least 2 points according to the MADRS item 6.

In an aspect, the invention relates to a D4 (and preferably also 5-HT2A) receptor antagonist, reverse agonist, or partial agonist, preferably pipamperone, preferably at a daily dose of 5 to 20 mg or 4 to 20 mg, for use in preventing, delaying or postponing the onset or progression, and/or treating, preferably for use in preventing, delaying or postponing the onset or progression of a cognitive disorder or an affective disorder, preferably major depressive disorder, which may be mild major depressive disorder, in a subject, preferably major (neuro)cognitive disorder or dementia, preferably caused by Alzheimer's disease, and/or for use in maintaining or improving cognitive function in a subject, and/or for preventing suicide or reducing suicidal thought in a subject, in particular a subject having (a medical history of) an affective disorder, in particular (mild) major depressive disorder.

In another aspect, the invention relates to a method for preventing, delaying or postponing the onset or progression, and/or treating, preferably for use in preventing, delaying or postponing the onset or progression of a cognitive disorder, preferably major (neuro)cognitive disorder or dementia, preferably caused by Alzheimer's disease, or an affective disorder, preferably (mild) major depressive disorder, in a subject and/or for maintaining or improving cognitive function in a subject and/or for preventing suicide or reducing suicidal thought in a subject, in particular a subject having (a medical history of) an affective disorder, in particular (mild) major depressive disorder comprising administering a D4 (and preferably also 5-HT2A) receptor antagonist, reverse agonist, preferably pipamperone, preferably at a daily dose of 5 to 20 mg or 4 to 20 mg.

In another aspect, the invention relates to the use of a D4 (and preferably also 5-HT2A) receptor antagonist, reverse agonist, or partial agonist, preferably pipamperone, preferably at a daily dose of 5 to 20 mg or 4 to 20 mg, for the manufacture of a medicament to prevent, delay or postpone the onset or progression, and/or treat, preferably to prevent, delay or postpone the onset or progression of a cognitive disorder, preferably major (neuro)cognitive disorder or dementia, preferably caused by Alzheimer's disease, or an affective disorder, preferably (mild) major depressive disorder, in a subject and/or to maintain or improve cognitive function in a subject and/or for preventing suicide or reducing suicidal thought in a subject, in particular a subject having (a medical history of) an affective disorder, in particular (mild) major depressive disorder.

In an aspect, the invention relates to a D4 (and preferably also 5-HT2A) receptor antagonist, reverse agonist, or partial agonist, preferably pipamperone, preferably at a daily dose of 5 to 20 mg or 4 to 20 mg, for use in treating an affective disorder, preferably (mild) major depressive disorder, in a subject, in particular for use in improving cognitive function and/or reducing suicidal thoughts in a subject having an affective disorder, preferably (mild) major depressive disorder.

Improvement of cognitive function and reduction of suicidal thoughts can be evaluated as described herein elsewhere, such as according to the MMSE (for cognitive function) or the MADRS (item 6 for cognitive function or item 10 for suicidal thoughts). Preferably, improvement or reduction refers respectively to an improvement or reduction of at least 20%, preferably at least 30%.

In another aspect, the invention relates to a D4 (and preferably also 5-HT2A) receptor antagonist, reverse agonist, or partial agonist, preferably pipamperone, preferably at a daily dose of 5 to 20 mg, or 4 to 20 mg, for use in increasing the MMSE score and/or decreasing the number of qualified clinical restlessness events (during sleep or at night) in a subject.

In another aspect, the invention relates to a method for increasing the MMSE score and/or decreasing the number of qualified clinical restlessness events (during sleep or at night) in a subject comprising administering a D4 (and preferably also 5-HT2A) receptor antagonist, reverse agonist, or partial agonist, preferably pipamperone, preferably at a daily dose of 5 to 20 mg, or 4 to 20 mg.

In another aspect, the invention relates to the use of a D4 (and preferably also 5-HT2A) receptor antagonist, reverse agonist, or partial agonist, preferably pipamperone, preferably at a daily dose of 5 to 20 mg, or 4 to 20 mg, for the manufacture of a medicament to increase the MMSE score and/or decrease the number of qualified clinical restlessness events (during sleep or at night) in a subject

In another aspect, the invention relates to a D4 (and preferably also 5-HT2A) receptor antagonist, reverse agonist, or partial agonist, preferably pipamperone, preferably at a daily dose of 5 to 20 mg, or 4 to 20 mg, for use in maintaining or increasing (relative) gamma power in a subject, preferably at the (frontal) cortex and/or during (REM) sleep.

In another aspect, the invention relates to a method for maintaining or increasing (relative) gamma power in a subject, preferably at the (frontal) cortex and/or during (REM) sleep, comprising administering a D4 (and preferably also 5-HT2A) receptor antagonist, reverse agonist, preferably pipamperone, preferably at a daily dose of 5 to 20 mg, or 4 to 20 mg.

In another aspect, the invention relates to the use of a D4 (and preferably also 5-HT2A) receptor antagonist, reverse agonist, or partial agonist, preferably pipamperone, preferably at a daily dose of 5 to 20 mg, or 4 to 20 mg, for the manufacture of a medicament to maintain or increase (relative) gamma power in a subject, preferably at the (frontal) cortex and/or during (REM) sleep.

In certain embodiments, the invention relates to a D4 (and preferably also 5-HT2A) receptor antagonist, reverse agonist, or partial agonist for use in preventing, delaying or postponing the onset or progression, and/or treating of a cognitive disorder in a subject, and/or for use in maintaining or improving cognitive function in a subject, and/or for maintaining or increasing (relative) gamma power in a subject, preferably at the (frontal) cortex and/or during (REM) sleep, which is, will be, or has been treated with transcranial direct current stimulation (tDCS).

In certain embodiments, the invention relates to a D4 (and preferably also 5-HT2A) receptor antagonist, reverse agonist, or partial agonist for use in preventing, delaying or postponing the onset or progression, and/or treating of a cognitive disorder in a subject, and/or for use in maintaining or improving cognitive function in a subject, and/or for maintaining or increasing (relative) gamma power in a subject, preferably at the (frontal) cortex and/or during (REM) sleep, simultaneously with or subsequent to, in either order, transcranial direct current stimulation (tDCS).

In certain embodiments, the invention relates to a method for preventing, delaying or postponing the onset or progression, and/or treating of a cognitive disorder in a subject, and/or for maintaining or improving cognitive function in a subject, and/or for maintaining or increasing (relative) gamma power in a subject, preferably at the (frontal) cortex and/or during (REM) sleep, comprising administering a D4 (and preferably also 5-HT2A) receptor antagonist, reverse agonist, or partial agonist simultaneously or sequentially in either order with transcranial direct current stimulation (tDCS).

In certain embodiments, the invention relates to the use of a D4 (and preferably also 5-HT2A) receptor antagonist, reverse agonist, or partial agonist for the manufacture of a medicament to prevent, delay or postpone the onset or progression, and/or treat of a cognitive disorder in a subject, and/or to maintain or improve cognitive function in a subject, and/or for maintaining or increasing (relative) gamma power in a subject, preferably at the (frontal) cortex and/or during (REM) sleep, which is, will be, or has been treated with transcranial direct current stimulation (tDCS).

In certain embodiments, the invention relates to the use of a D4 (and preferably also 5-HT2A) receptor antagonist, reverse agonist, or partial agonist for the manufacture of a medicament to prevent, delay or postpone the onset or progression, and/or treat of a cognitive disorder in a subject, and/or to maintain or improve cognitive function in a subject, and/or for maintaining or increasing (relative) gamma power in a subject, preferably at the (frontal) cortex and/or during (REM) sleep, simultaneously with or subsequent to, in either order, transcranial direct current stimulation (tDCS).

In certain embodiments, the invention relates to a D4 (and preferably also 5-HT2A) receptor antagonist, reverse agonist, or partial agonist for use in increasing the MMSE score and/or decreasing the number of qualified clinical restlessness events (during sleep or at night) in a subject, and/or for maintaining or increasing (relative) gamma power in a subject, preferably at the (frontal) cortex and/or during (REM) sleep, which is, will be, or has been treated with tDCS.

In certain embodiments, the invention relates to a D4 (and preferably also 5-HT2A) receptor antagonist, reverse agonist, or partial agonist for use in increasing the MMSE score and/or decreasing the number of qualified clinical restlessness events (during sleep or at night) in a subject, and/or for maintaining or increasing (relative) gamma power in a subject, preferably at the (frontal) cortex and/or during (REM) sleep, simultaneously with or subsequent to, in either order, tDCS.

In certain embodiments, the invention relates to a method for increasing the MMSE score and/or decreasing the number of qualified clinical restlessness events (during sleep or at night) in a subject, and/or for maintaining or increasing (relative) gamma power in a subject, preferably at the (frontal) cortex and/or during (REM) sleep, comprising administering a D4 (and preferably also 5-HT2A) receptor antagonist, reverse agonist, or partial agonist simultaneously with or subsequent to, in either order, tDCS.

In certain embodiments, the invention relates to the use of a D4 (and preferably also 5-HT2A) receptor antagonist, reverse agonist, or partial agonist for the manufacture of a medicament to increase the MMSE score and/or decrease the number of qualified clinical restlessness events during sleep in a subject, and/or for maintaining or increasing (relative) gamma power in a subject, preferably at the (frontal) cortex and/or during (REM) sleep, which is, will be, or has been treated with tDCS.

In certain embodiments, the invention relates to the use of a D4 (and preferably also 5-HT2A) receptor antagonist, reverse agonist, or partial agonist for the manufacture of a medicament to increase the MMSE score and/or decrease the number of qualified clinical restlessness events (during sleep or at night) in a subject, and/or for maintaining or increasing (relative) gamma power in a subject, preferably at the (frontal) cortex and/or during (REM) sleep, simultaneously with or subsequent to, in either order, tDCS.

In certain embodiments, the invention relates to transcranial direct current stimulation (tDCS) for use in preventing, delaying or postponing the onset or progression, and/or treating of a cognitive disorder in a subject, and/or for use in maintaining or improving cognitive function in a subject, and/or for maintaining or increasing (relative) gamma power in a subject, preferably at the (frontal) cortex and/or during (REM) sleep, which is, will be, or has been treated with a D4 (and preferably also 5-HT2A) receptor antagonist, reverse agonist, or partial agonist.

In certain embodiments, the invention relates to transcranial direct current stimulation (tDCS) for use in preventing, delaying or postponing the onset or progression, and/or treating of a cognitive disorder in a subject, and/or for use in maintaining or improving cognitive function in a subject, and/or for maintaining or increasing (relative) gamma power in a subject, preferably at the (frontal) cortex and/or during (REM) sleep, simultaneously with or subsequent to, in either order, a D4 (and preferably also 5-HT2A) receptor antagonist, reverse agonist, or partial agonist.

In certain embodiments, the invention relates to a method for preventing, delaying or postponing the onset or progression, and/or treating of a cognitive disorder in a subject, and/or for maintaining or improving cognitive function in a subject, and/or for maintaining or increasing (relative) gamma power in a subject, preferably at the (frontal) cortex and/or during (REM) sleep, comprising administering, applying, or performing transcranial direct current stimulation (tDCS) simultaneously or sequentially, in either order, with administration of a D4 (and preferably also 5-HT2A) receptor antagonist, reverse agonist, or partial agonist.

In certain embodiments, the invention relates to tDCS for use in increasing the MMSE score and/or decreasing the number of qualified clinical restlessness events (during sleep or at night) in a subject, and/or for maintaining or increasing (relative) gamma power in a subject, preferably at the (frontal) cortex and/or during (REM) sleep, which is, will be, or has been treated with a D4 (and preferably also 5-HT2A) receptor antagonist, reverse agonist, or partial agonist.

In certain embodiments, the invention relates to tDCS for use in increasing the MMSE score and/or decreasing the number of qualified clinical restlessness events (during sleep or at night) in a subject, and/or for maintaining or increasing (relative) gamma power in a subject, preferably at the (frontal) cortex and/or during (REM) sleep, simultaneously with or subsequent to, in either order, a D4 (and preferably also 5-HT2A) receptor antagonist, reverse agonist, or partial agonist.

In certain embodiments, the invention relates to a method for increasing the MMSE score and/or decreasing the number of qualified clinical restlessness events (during sleep or at night) in a subject, and/or for maintaining or increasing (relative) gamma power in a subject, preferably at the (frontal) cortex and/or during (REM) sleep, comprising administering, applying, or performing tDCS simultaneously with or subsequent to, in either order, a D4 (and preferably also 5-HT2A) receptor antagonist, reverse agonist, or partial agonist.

In certain embodiments, the invention relates to tDCS for use in preventing, delaying or postponing the onset or progression, and/or treating of a cognitive disorder in a subject, and/or for use in maintaining or improving cognitive function in a subject, and/or for maintaining or increasing (relative) gamma power in a subject, preferably at the (frontal) cortex and/or during (REM) sleep.

In certain embodiments, the invention relates to a method for preventing, delaying or postponing the onset or progression, and/or treating of a cognitive disorder in a subject and/or for maintaining or improving cognitive function in a subject, and/or for maintaining or increasing (relative) gamma power in a subject, preferably at the (frontal) cortex and/or during (REM) sleep, comprising applying tDCS.

In certain embodiments, the invention relates to tDCS for use in increasing the MMSE score and/or decreasing the number of qualified clinical restlessness events (during sleep or at night) in a subject, and/or for maintaining or increasing (relative) gamma power in a subject, preferably at the (frontal) cortex and/or during (REM) sleep.

In certain embodiments, the invention relates to a method for increasing the MMSE score and/or decreasing the number of qualified clinical restlessness events (during sleep or at night) in a subject, and/or for maintaining or increasing (relative) gamma power in a subject, preferably at the (frontal) cortex and/or during (REM) sleep, comprising applying tDCS.

In certain embodiments, the invention relates to a D4 (and preferably also 5-HT2A) receptor antagonist, reverse agonist, or partial agonist as an adjunctive therapy to tDCS for use in increasing the MMSE score and/or decreasing the number of qualified clinical restlessness events (during sleep or at night) in a subject, and/or for maintaining or increasing (relative) gamma power in a subject, preferably at the (frontal) cortex and/or during (REM) sleep.

In certain embodiments, the invention relates to a D4 (and preferably also 5-HT2A) receptor antagonist, reverse agonist, or partial agonist for use as an adjunctive therapy to tDCS to increase the MMSE score and/or decreasing the number of qualified clinical restlessness events (during sleep or at night) in a subject, and/or for maintaining or increasing (relative) gamma power in a subject, preferably at the (frontal) cortex and/or during (REM) sleep.

In certain embodiments, the invention relates to a method for increasing the MMSE score and/or decreasing the number of qualified clinical restlessness events (during sleep or at night) in a subject, and/or for maintaining or increasing (relative) gamma power in a subject, preferably at the (frontal) cortex and/or during (REM) sleep, comprising administering a D4 (and preferably also 5-HT2A) receptor antagonist, reverse agonist, or partial agonist as an adjunctive therapy to tDCS.

In certain embodiments, the invention relates to the use of a D4 (and preferably also 5-HT2A) receptor antagonist, reverse agonist, or partial agonist for the manufacture of a medicament as an adjunctive therapy to tDCS to increase the MMSE score and/or decrease the number of qualified clinical restlessness events (during sleep or at night) in a subject, and/or for maintaining or increasing (relative) gamma power in a subject, preferably at the (frontal) cortex and/or during (REM) sleep.

In certain embodiments, the invention relates to a D4 (and preferably also 5-HT2A) receptor antagonist, reverse agonist, or partial agonist as an adjunctive therapy to tDCS for use in preventing, delaying or postponing the onset or progression, or treating a cognitive disorder in a subject, and/or for use in maintaining or improving cognitive function in a subject, and/or for maintaining or increasing (relative) gamma power in a subject, preferably at the (frontal) cortex and/or during (REM) sleep.

In certain embodiments, the invention relates to a D4 (and preferably also 5-HT2A) receptor antagonist, reverse agonist, or partial agonist for use as an adjunctive therapy to tDCS to prevent, delay or postpone the onset or progression, or treat a cognitive disorder in a subject, and/or to maintain or improve cognitive function in a subject, and/or for maintaining or increasing (relative) gamma power in a subject, preferably at the (frontal) cortex and/or during (REM) sleep.

In certain embodiments, the invention relates to a method for preventing, delaying or postponing the onset or progression, or treating a cognitive disorder in a subject, and/or for maintaining or improving cognitive function in a subject, and/or for maintaining or increasing (relative) gamma power in a subject, preferably at the (frontal) cortex and/or during (REM) sleep, comprising administering a D4 (and 5-HT2A) receptor antagonist, reverse agonist, or partial agonist as an adjunctive therapy to tDCS.

In certain embodiments, the invention relates to the use of a D4 (and preferably also 5-HT2A) receptor antagonist, reverse agonist, or partial agonist for the manufacture of a medicament as an adjunctive therapy to tDCS to prevent, delay or postpone the onset or progression, or treat a cognitive disorder in a subject, and/or to maintain or improve cognitive function in a subject, and/or for maintaining or increasing (relative) gamma power in a subject, preferably at the (frontal) cortex and/or during (REM) sleep.

It will be understood that in embodiments where subjects are treated simultaneously with the D4 (and preferably also 5-HT2A) receptor antagonist, reverse agonist, or partial agonist and tDCS need not necessarily be treated at the exact same time with the D4 (and 5-HT2A) receptor antagonist, reverse agonist, or partial agonist and tDCS. For instance, in a single day, but at different times a subject may be treated with the D4 (and 5-HT2A) receptor antagonist, reverse agonist, or partial agonist and tDCS. Simultaneous treatment may also encompass treatment with both the D4 (and 5-HT2A) receptor antagonist, reverse agonist, or partial agonist and tDCS during a predefined period, but not necessarily at the same day. For instance, a subject may be treated for a defined period daily with the D4 (and 5-HT2A) receptor antagonist, reverse agonist, or partial agonist and every other day with tDCS; or a subject may be treated for a defined period every other day with the D4 (and 5-HT2A) receptor antagonist, reverse agonist, or partial agonist and on intermittent days with tDCS.

In certain embodiments, the aspects described above both encompass treatment with the D4 (and 5-HT2A) receptor antagonist, reverse agonist, or partial agonist and tDCS. In certain embodiments, the aspects described above only encompass treatment with the D4 (and 5-HT2A) receptor antagonist, reverse agonist, or partial agonist, while treatment with tDCS does not form part of the subject-matter of the aspect per se, but rather defines the patient population. Accordingly, when referring herein to a “subject”, the subject may be defined as a subject having undergone, undergoing, or scheduled to undergo treatment with tDCS.

As used herein, the subject to be treated according to the invention may be a human subject but is not restricted to humans. A subject to be treated according to the invention may also include other mammals, for instance, domestic animals.

As used herein, the terms “D4 (and preferably also 5-HT2A) receptor antagonist, reverse agonist, or partial agonist” or “D4 (and 5-HT2A) receptor antagonist, reverse agonist, or partial agonist” refer to a compound, such as a pharmaceutically active ingredient, which is an antagonist, reverse agonist, or partial agonist of the dopamine D4 receptor and optionally but preferably the serotonin 5-HT2A receptor. Preferably, such compound has a selective affinity for D4 and optionally a selective affinity for the 5-HT2A receptor. By the expression “selective affinity for the 5-HT2A receptor” is meant that the receptor has a higher affinity for the 5-HT2A receptor than for other 5-HT receptors. The expression “selective affinity for the D4 receptor” means that the receptor has a higher affinity for the dopamine D4 receptor than for other dopamine receptors. Preferably, such compound has an affinity for the D4 receptor with a pKi value equal or higher than 8 towards the D4 receptor and/or has an affinity for the 5-HT2A receptor with a pKi value equal or higher than 8 towards the 5-HT2A receptor. Preferably, such compound has a selective affinity for the D4 receptor with a pKi value equal or higher than 8 towards the D4 receptor and/or has a selective affinity for the 5-HT2A receptor with a pKi value equal or higher than 8 towards the 5-HT2A receptor. Preferably, compound has an affinity for the D4 receptor with a pKi value equal or higher than 8 towards the D4 receptor and less than 8 towards other dopamine receptors and/or has an affinity for the 5-HT2A receptor with a pKi value equal or higher than 8 towards the 5-HT2A receptor and less than 8 towards other serotonin receptors. Preferably, compound has a selective affinity for the D4 receptor with a pKi value equal or higher than 8 towards the D4 receptor and less than 8 towards other dopamine receptors and/or has a selective affinity for the 5-HT2A receptor with a pKi value equal or higher than 8 towards the 5-HT2A receptor and less than 8 towards other serotonin receptors. In certain embodiments, the pKi of the D4 (and 5-HT2A) receptor antagonist, reverse agonist, or partial agonist is at least one unit higher for the D4 and/or 5-HT2A receptor(s) than (respectively) for other dopamine and/or serotonin receptors. The term “other 5HT receptors” as used herein refers to, for instance, 5-HT1 receptors (e.g. 5-HT1A, 5-HT1B, 5-HT1D, 5-HT1E, 5-HT1F), 5-HT2B, 5-HT2C, 5-HT6 (rat) and 5-HT7 (rat). The term “other dopamine receptors” as used herein refers to, for instance, D1, D2 and D3 dopamine receptors. In certain preferred embodiments, the D4 (and 5-HT2A) receptor antagonist, reverse agonist, or partial agonist is a D4 (and 5-HT2A) receptor antagonist. The selectivity of the D4 (and 5-HT2A) receptor antagonist, reverse agonist, or partial agonist for the respective receptors may be dose dependent. It will be understood that the D4 (and 5-HT2A) receptor antagonist, reverse agonist, or partial agonist preferably is or is to be administered at a dose appropriate to ensure D4 (and 5-HT2A) receptor antagonism, reverse agonism, or partial agonism, preferably while at the same time not exerting such effects on other receptors such as other dopamine or serotonin receptors. Accordingly, in certain embodiments the dose of the D4 (and 5-HT2A) receptor antagonist, reverse agonist, or partial agonist, such as the daily dose, is chosen such that the D4 (and 5-HT2A) receptor antagonist, reverse agonist, or partial agonist has an affinity for the D4 receptor with a pKi value equal or higher than 8 towards the D4 receptor and less than 8 towards other dopamine receptors and/or has a selective affinity for the 5-HT2A receptor with a pKi value equal or higher than 8 towards the 5-HT2A receptor and less than 8 towards other serotonin receptors.

In certain embodiments, the expression “the D4 and 5-HT2A antagonist, inverse agonist or partial agonist” is used herein to indicate a single compound having both activities or alternatively to indicate a composition combining the activities of separate compounds (such as a first compound comprising selective D4 receptor antagonism, inverse agonism, or partial agonism in combination with a second compound comprising selective 5-HT2A receptor antagonism, reverse agonism, or partial agonism). Accordingly, when the 5-HT2A and D4 antagonist, inverse agonist or partial agonist activity reside in separate compounds, the term “composition” may be used.

As used herein, the term “agonist” relates to an agent which both binds to a receptor and has an intrinsic effect. An agonist binds to and activates a receptor. This can be be full, partial or inverse. A full agonist has high efficacy, producing a full response while occupying a relatively low proportion of receptors. A partial agonist has lower efficacy than a full agonist. It produces sub-maximal activation even when occupying the total receptor population, therefore cannot produce the maximal response, irrespective of the concentration applied. An inverse agonist produces an effect opposite to that of an agonist, yet binds to the same receptor binding-site as an agonist.

As used herein, the term “antagonist” refers to an interaction between chemicals in which one partially or completely inhibits the effect of the other, in particular agents having high affinity for a given receptor, but which do not activate this receptor and preferably inhibit activation of the receptor. An antagonist attenuates the effect of an agonist. This can be competitive or non-competitive, each of which can be reversible or irreversible. A competitive antagonist binds to the same site as the agonist but does not activate it, thus blocks the agonist's action. A non-competitive antagonist binds to an allosteric (non-agonist) site on the receptor to prevent activation of the receptor. A reversible antagonist binds non-covalently to the receptor, therefore can be “washed out”. An irreversible antagonist cannot be displaced by either competing ligands or washing, either by forming a covalent bond to or near the active site, or alternatively just by binding so tightly that the rate of dissociation is effectively zero at relevant time scales.

As used herein, the term pKi refers to the negative logarithm (base 10) of the Ki. “Ki” refers to the inhibition constant for a ligand, which denotes the affinity of the ligand for a receptor. Measured using a radioligand competition binding assay, it is the molar concentration of the competing ligand that would occupy 50% of the receptors if no radioligand was present. It is calculated from the IC50 value using the Cheng-Prusoff equation. In a functional assay, IC50 refers to the molar concentration of an agonist or antagonist which produces 50% of its maximum possible inhibition. In a radioligand binding assay, the molar concentration of competing ligand which reduces the specific binding of a radioligand by 50%. The Cheng-Prusoff Equation used to determine the Ki value from an IC50 value measured in a competition radioligand binding assay:

$$Ki = \frac{IC 50}{1 + \frac{[ L ]}{Kd}}$$

Where [L] is the concentration of free radioligand, and Kd is the dissociation constant of the radioligand for the receptor. The dissociation constant for a radiolabeled drug is determined by saturation analysis. It is the molar concentration of radioligand which, at equilibrium, occupies 50% of the receptors.

Ki values of test compounds for dopamine receptors as well as 5-HT2A receptors can be measured using commonly known assays or can be consulted in databases. For instance, the “NIMH Psychoactive Drug Screening Program (PDSP)” Ki database (https://pdsp.unc.edu/databases/kidb.php) is a unique resource in the public domain which provides information on the abilities of drugs to interact with an expanding number of molecular targets. The PDSP Ki database serves as a data warehouse for published and internally-derived pKi, or affinity, values fora large number of drugs and drug candidates at an expanding number of G-protein coupled receptors, ion channels, transporters and enzymes. The PDSP internet site also provides for commonly used protocols and assays for measuring pKi values of 5-HT and dopamine receptors.

In certain embodiments, the D4 (and 5-HT2A) receptor antagonist, reverse agonist, or partial agonist is or is to be administered daily. In certain embodiments, the D4 (and 5-HT2A) receptor antagonist, reverse agonist, or partial agonist is to be administered multiple times per day. In certain embodiments, the D4 (and 5-HT2A) receptor antagonist, reverse agonist, or partial agonist is or is to be administered ad noctum (i.e. in the evening, before going to sleep). In certain embodiments, the D4 (and 5-HT2A) receptor antagonist, reverse agonist, or partial agonist is to be administered as multiple partial doses per day. In certain embodiments, the D4 (and 5-HT2A) receptor antagonist, reverse agonist, or partial agonist is to be administered once per several days, such as once per two or three days. It will be understood that in such case, also a “theoretical” daily dose can be calculated (e.g. divide the dose administered by the number of days between consecutive administrations). Also in such discontinuous administration regime (i.e. not daily administration) the dose may be split in partial doses. Preferably however, the D4 (and 5-HT2A) receptor antagonist, reverse agonist, or partial agonist is or is to be administered daily, either as a single dose, or as multiple partial doses.

The skilled person will understand that that the D4 (and 5-HT2A) receptor antagonist, reverse agonist, or partial agonist may need to be administered indefinitely, such as lifelong. However, in certain cases, the D4 (and 5-HT2A) receptor antagonist, reverse agonist, or partial agonist may be (possibly only for a certain period) discontinued once a particular desired therapeutic effect is achieved, such as for instance a particular improvement in cognitive function(s), as can be determined for instance with MMSE as described herein elsewhere.

In certain embodiments, the D4 (and 5-HT2A) receptor antagonist, reverse agonist, or partial agonist is a D4 receptor antagonist, reverse agonist, or partial agonist. Preferably however, the D4 (and 5-HT2A) receptor antagonist, reverse agonist, or partial agonist is a D4 and 5-HT2A receptor antagonist, reverse agonist, or partial agonist.

In a preferred embodiment the D4 and 5-HT2A receptor antagonist, reverse agonist, or partial agonist is pipamperone.

Pipamperone (1′-[3-(p-fluorobenzoyl)propyl]-[1,4′-bipiperidine]-4′-carboxamide) or 1-[4-(4-fluorophenyl)-4-oxobutyl]-4-piperidin-1-ylpiperidine-4-carboxamide), also known as carpiperone and floropipamide or fluoropipamide, and as floropipamide hydrochloride, is a typical antipsychotic of the butyrophenone family used in the treatment of schizophrenia. It is or has been marketed under brand names including Dipiperon, Dipiperal, Piperonil, Piperonyl, and Propitan. The chemical formula of pipamperone is provided below.

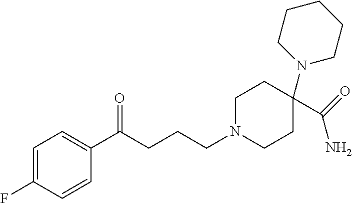 [SMILES: NC(=O)C1(N2CCCCC2)CCN(CCCC(=O)c2ccc(F)cc2)CC1]

It will be understood that pharmaceutically acceptable derivatives, such as salts are also included. In certain embodiments, pipamperone is pipamperone (di)hydrochloride (pipamperone HCl). In will be understood that when pipamperone is formulated as a pharmaceutically acceptable derivative, such as a salt, such as pipamperone HCl, the dose may be adjusted to the equivalent of pipamperone.

Pipamperone acts as an antagonist of the 5-HT2A, 5-HT2B, 5-HT2C, D2, D3, D4, α1-adrenergic, and a2-adrenergic receptors. It shows much higher affinity for the 5-HT2A and D4 receptors over the D2 receptor (15-fold in the case of the D4 receptor, and even higher in the case of the 5-HT2A receptor), being regarded as “highly selective” for the former two sites at low doses. Pipamperone has low and likely insignificant affinity for the H1 and mACh receptors, as well as for other serotonin and dopamine receptors.

In certain embodiments, pipamperone is or is to be administered in a daily dose ranging from 2.5 to 30 mg, preferably 5 to 20 mg, or 4 to 20 mg, such as 4, 5, 6, 7, 8, 9, 10, 11, 12, 13, 14, 15, 16, 17, 18, 19, or 20 mg. In certain embodiments, pipamperone is or is to be administered in a daily dose ranging from 4 to 15 mg. In certain embodiments, pipamperone is or is to be administered in a daily dose ranging from 5 to 15 mg. In certain embodiments, pipamperone is or is to be administered in a daily dose ranging from 10 to 20 mg. In certain embodiments, pipamperone is or is to be administered in a daily dose ranging from 15 to 20 mg. The daily dose may be administered at once or alternatively may be administered as multiple partial doses, such as bi-daily administration or tridaily administration, whereby the multiple partial doses may be the same or different. In the dose range from 4 or 5 to 20 mg daily, pipamperone exhibits selective D4 and 5-HT2A receptor antagonism, while not exhibiting significant antagonism towards other dopamine and serotonin receptors.

In certain other embodiments the D4 (and 5-HT2A) receptor antagonist, reverse agonist, or partial agonist is selected from L-745,870, NGD 94-1, PNU-101,387, CP-293,019, PD-172,938, U-101,958, S-18126, NRA0045, NRA0160.

As used herein, the term “cognitive disorder” also known as “neurocognitive disorder” refers to a category of mental health disorders that primarily affect cognitive abilities including learning, memory, perception, and problem solving. Neurocognitive disorders include delirium and mild and major neurocognitive disorder (also known as dementia). They are defined by deficits in cognitive ability that are acquired (as opposed to developmental), typically represent decline, and may have an underlying brain pathology. The DSM-5 defines six key domains of cognitive function: executive function, learning and memory, perceptual-motor function, language, complex attention, and social cognition. Neurocognitive disorders are described in DSM-5 as those with “a significant impairment of cognition or memory that represents a marked deterioration from a previous level of function”.

Although Alzheimer's disease accounts for the majority of cases of neurocognitive disorders, there are various medical conditions that affect mental functions such as memory, thinking, and the ability to reason, including frontotemporal degeneration, Huntington's disease, Lewy body disease, traumatic brain injury (TBI), Parkinson's disease, prion disease, and dementia/neurocognitive issues due to HIV infection. Neurocognitive disorders are diagnosed as mild and major based on the severity of their symptoms. While anxiety disorders, mood disorders, and psychotic disorders can also have an effect on cognitive and memory functions, the DSM-5 does not consider these cognitive disorders, because loss of cognitive function is not the primary (causal) symptom. Additionally, developmental disorders such as Autism spectrum disorder are typically developed at birth or early in life as opposed to the acquired nature of neurocognitive disorders.

Delirium develops rapidly over a short period of time and is characterized by a disturbance in cognition, manifested by confusion, excitement, disorientation, and a clouding of consciousness. Hallucinations and illusions are common, and some individuals may experience acute onset change of consciousness. It is a disorder that makes situational awareness and processing new information very difficult for those diagnosed. It usually has a high rate of onset ranging from minutes to hours and sometimes days, but it does not last for very long, only a few hours to weeks. Delirium can also be accompanied by a shift in attention, mood swings, violent or unordinary behaviours, and hallucinations. It can be caused by a pre-existing medical condition. Delirium during a hospital stay can result in a longer stay and more risk of complications and long terms stays.

Mild and major neurocognitive disorders are usually associated with but not restricted to the elderly. Unlike delirium, conditions under these disorders develop slowly and are characterized by memory loss. In addition to memory loss and cognitive impairment, other symptoms include aphasia, apraxia, agnosia, loss of abstract thought, behavioural/personality changes, and impaired judgment. There may also be behavioural disturbances including psychosis, mood, and agitation.

Mild and major neurocognitive disorders are differentiated based on the severity of their symptoms. Previously known as dementia, major neurocognitive disorder is characterized by significant cognitive decline and interference with independence, while mild neurocognitive disorder is characterized by moderate cognitive decline and does not interfere with independence. To be diagnosed, it must not be due to delirium or other mental disorder. They are also usually accompanied by another cognitive dysfunction. For non-reversible causes of dementia such as age, the slow decline of memory and cognition is lifelong. It can be diagnosed by screening tests such as the Mini Mental State Examination (MMSE).

Neurocognitive disorders can have numerous causes: genetics, brain trauma, stroke, metabolic, and heart issues. The main causes are neurodegenerative diseases such as Alzheimer's disease, Parkinson's disease, and Huntington's disease because they affect or deteriorate brain functions.

Other diseases and conditions that cause NDCs include vascular dementia, frontotemporal degeneration, Lewy body disease, prion disease, normal pressure hydrocephalus, and dementia/neurocognitive issues due to HIV infection. They may also include dementia due to substance abuse or exposure to toxins.

Neurocognitive disorder may also be caused by brain trauma, including concussions and Traumatic Brain Injuries, as well as post-traumatic stress and alcoholism. This is referred to as amnesia, and is characterized by damage to major memory encoding parts of the brain such as the hippocampus. Difficulty creating recent term memories is called anterograde amnesia and is caused by damage to the hippocampus part of the brain, which is a major part of the memory process. Retrograde amnesia is also caused by damage to the hippocampus, but the memories that were encoded or in the process of being encoded in long term memory are erased.

Preferably, the cognitive disorder as used herein is a major neurocognitive disorder or dementia, preferably as defined according to DSM-5 (Diagnostic and Statistical Manual of Mental Disorders).

In certain embodiments, the cognitive disorder is caused by a neurodegenerative disease, preferably a chronic neurodegenerative disease. In certain embodiments, the (major) neurocognitive disorder or dementia is caused by a neurodegenerative disease, preferably a chronic neurodegenerative disease such as Alzheimer's disease.

Major neurocognitive disorder or dementia is a broad category of brain diseases that cause a long-term and often gradual decrease in the ability to think and remember that is great enough to affect a person's daily functioning. Other common symptoms include emotional problems, difficulties with language, and a decrease in motivation. A person's consciousness is usually not affected. A dementia diagnosis requires a change from a person's usual mental functioning and a greater decline than one would expect due to aging.

The most common type of dementia is (caused by) Alzheimer's disease, which makes up 50% to 70% of cases. Other common types include vascular dementia (25%), Lewy body dementia (15%), and frontotemporal dementia. Less common causes include normal pressure hydrocephalus, Parkinson's disease dementia, syphilis, and Creutzfeldt-Jakob disease among others. More than one type of dementia may exist in the same person. A small proportion of cases run in families. In the DSM-5, dementia was reclassified as a neurocognitive disorder, with various degrees of severity. Diagnosis is usually based on history of the illness and cognitive testing with medical imaging and blood tests used to rule out other possible causes. The mini mental state examination is one commonly used cognitive test.

The symptoms of dementia vary across types and stages of the diagnosis. The most common affected areas include memory, visual-spatial, language, attention and problem solving. Most types of dementia are slow and progressive. By the time the person shows signs of the disorder, the process in the brain has been happening for a long time. It is possible for a patient to have two types of dementia at the same time. About 10% of people with dementia have what is known as mixed dementia, which is usually a combination of Alzheimer's disease and another type of dementia such as frontotemporal dementia or vascular dementia.

In the early stage of dementia, the person begins to show symptoms noticeable to the people around them. In addition, the symptoms begin to interfere with daily activities. The person usually scores between a 20 and 25 on the MMSE. The symptoms are dependent on the type of dementia a person has. The person may begin to have difficulty with more complicated chores and tasks around the house or at work. The person can usually still take care of him or herself but may forget things like taking pills or doing laundry and may need prompting or reminders.

The symptoms of early dementia usually include memory difficulty, but can also include some word-finding problems (anomia) and problems with planning and organizational skills (executive function). One very good way of assessing a person's impairment is by asking if they are still able to handle their finances independently. This is often one of the first things to become problematic. Other signs might be getting lost in new places, repeating things, personality changes, social withdrawal and difficulties at work.

When evaluating a person for dementia, it is important to consider how the person was able to function five or ten years earlier. It is also important to consider a person's level of education when assessing for loss of function. For example, an accountant who can no longer balance a checkbook would be more concerning than a person who had not finished high school or had never taken care of his/her own finances.

In Alzheimer's dementia the most prominent early symptom is memory difficulty. Others include word-finding problems and getting lost. In other types of dementia, like dementia with Lewy bodies and frontotemporal dementia, personality changes and difficulty with organization and planning may be the first signs.

As dementia progresses, the symptoms first experienced in the early stages of the dementia generally worsen. The rate of decline is different for each person. A person with moderate dementia typically scores between 10-18 on the MMSE. For example, people with Alzheimer's dementia in the moderate stages lose almost all new information very quickly. People with dementia may be severely impaired in solving problems, and their social judgment is usually also impaired. They cannot usually function outside their own home, and generally should not be left alone. They may be able to do simple chores around the house but not much else, and begin to require assistance for personal care and hygiene other than simple reminders.

People with late-stage dementia typically turn increasingly inward and need assistance with most or all of their personal care. Persons with dementia in the late stages usually need 24-hour supervision to ensure personal safety, as well as to ensure that basic needs are being met. If left unsupervised, a person with late-stage dementia may wander or fall, may not recognize common dangers around them such as a hot stove, may not realize that they need to use the bathroom or become unable to control their bladder or bowels (incontinent).

Changes in eating frequently occur. Caregivers of people with late-stage dementia often provide pureed diets, thickened liquids, and assistance in eating, to prolong their lives, to cause them to gain weight, to reduce the risk of choking, and to make feeding the person easier. The person's appetite may decline to the point that the person does not want to eat at all. They may not want to get out of bed, or may need complete assistance doing so. Commonly, the person no longer recognizes familiar people. They may have significant changes in sleeping habits or have trouble sleeping at all.

In certain embodiments, the cognitive disorder is selected from (dementia caused by) Alzheimer Disease, dementia, substance-related persisting dementia, vascular dementia, dementia due to HIV disease, dementia due to head trauma, dementia due to Parkinson Disease, dementia due to Huntington Disease, dementia due to Pick Disease, dementia due to Creutzfedt-Jacob Disease, amnestic disorders due to a general medical condition, substance-induced persisting amnestic disorder, mild cognitive impairment disorder, or delirium.

In certain embodiments, the cognitive disorder is selected from major cognitive disorder, mild cognitive disorder, or delirium.

In certain embodiments, the cognitive disorder is selected from major cognitive disorder or mild cognitive disorder.

In certain embodiments, the cognitive disorder is major cognitive disorder.

In certain embodiments, the cognitive disorder is selected from (dementia caused by) Alzheimer's disease, Huntington's Disease, Parkinson's Disease, or Pick Disease.

In certain embodiments, the cognitive disorder is dementia caused by or associated with Alzheimer's disease, Huntington's Disease, Parkinson's Disease, or Pick Disease.

In certain embodiments, the (major) neurocognitive disorder is or is (dementia) caused by Alzheimer's Disease, substance-related persisting dementia, vascular dementia, dementia due to HIV disease, dementia with Lewy bodies, dementia due to head trauma/chronic traumatic encephalopathy, dementia due to Parkinson Disease, dementia due to Huntington Disease, dementia due to Pick Disease (frontotemporal dementia), dementia due to prions (e.g. Creutzfedt-Jacob Disease), Normal Pressure Hydrocephalus, subdural hematoma, posterior cortical atrophy, corticobasal degeneration, progressive supranuclear palsy, mixed dementia, medication side effects, chronic alcoholism, tumors or infections in the brain, toxins (e.g. lead), Wernicke-Korsakoff, hypothyroidism, vitamin B12 deficiency, Lyme disease, and neurosyphillis, Behcet's disease, multiple sclerosis, sarcoidosis, Sjögren's syndrome, systemic lupus erythematosus, celiac disease, and non-celiac gluten sensitivity, vitamin B12 deficiency, folate deficiency, niacin deficiency, and infective causes including cryptococcal meningitis, AIDS, Lyme disease, progressive multifocal leukoencephalopathy, subacute sclerosing panencephalitis, syphilis, and Whipple's disease, Alexander disease, Canavan disease, Cerebrotendinous xanthomatosis, Dentatorubral-pallidoluysian atrophy, Epilepsy, Fatal familial insomnia, Fragile X-associated tremor/ataxia syndrome, Glutaric aciduria type 1, Krabbe's disease, Maple syrup urine disease, Niemann-Pick disease type C, Neuronal ceroid lipofuscinosis, Neuroacanthocytosis, Organic acidemias, Pelizaeus-Merzbacher disease, Sanfilippo syndrome type B, Spinocerebellar ataxia type 2, Urea cycle disorders, amnestic disorders due to a general medical condition, substance-induced persisting amnestic disorder, mild cognitive impairment disorder.

In certain embodiments, the (major) neurocognitive disorder is or is (dementia) caused by Alzheimer's Disease, vascular dementia, dementia with Lewy bodies, dementia due to Parkinson Disease, dementia due to Huntington Disease, dementia due to Pick Disease (frontotemporal dementia), posterior cortical atrophy, corticobasal degeneration, progressive supranuclear palsy.

In certain embodiments, the (major) neurocognitive disorder is or is (dementia) caused by Alzheimer's Disease, vascular dementia, dementia with Lewy bodies, or dementia due to Pick Disease (frontotemporal dementia).

In a preferred embodiment, the (major) neurocognitive disorder is or is (dementia) caused by Alzheimer's Disease. In a preferred embodiment, the cognitive disorder is or is (dementia) caused by Alzheimer's Disease. In a preferred embodiment, the cognitive disorder is Alzheimer's Disease. In a preferred embodiment, the cognitive disorder is dementia caused by Alzheimer's Disease.

Alzheimer's disease (AD), also referred to simply as Alzheimer's, is a chronic neurodegenerative disease that usually starts slowly and worsens over time. It is the cause of 60-70% of cases of dementia. The most common early symptom is difficulty in remembering recent events (short-term memory loss). As the disease advances, symptoms can include problems with language, disorientation (including easily getting lost), mood swings, loss of motivation, not managing self-care, and behavioural issues. As a person's condition declines, they often withdraw from family and society. Gradually, bodily functions are lost, ultimately leading to death. Although the speed of progression can vary, the typical life expectancy following diagnosis is three to nine years. The cause of Alzheimer's disease is poorly understood. About 70% of the risk is believed to be genetic with many genes usually involved. Other risk factors include a history of head injuries, depression, or hypertension. The disease process is associated with plaques and tangles in the brain. A probable diagnosis is based on the history of the illness and cognitive testing with medical imaging and blood tests to rule out other possible causes.

Vascular dementia, also known as multi-infarct dementia (MID) and vascular cognitive impairment (VCI), is dementia caused by problems in the supply of blood to the brain, typically a series of minor strokes, leading to worsening cognitive decline that occurs step by step. The term refers to a syndrome consisting of a complex interaction of cerebrovascular disease and risk factors that lead to changes in the brain structures due to strokes and lesions, and resulting changes in cognition. The temporal relationship between a stroke and cognitive deficits is needed to make the diagnosis. People with vascular dementia present with progressive cognitive impairment, acutely or subacutely as in mild cognitive impairment, frequently step-wise, after multiple cerebrovascular events. Vascular dementia can be caused by ischemic or hemorrhagic infarcts affecting multiple brain areas, including the anterior cerebral artery territory, the parietal lobes, or the cingulate gyrus. On rare occasion, infarcts in the hippocampus or thalamus are the cause of dementia.

Dementia with Lewy bodies (DLB) is a type of dementia accompanied by changes in behaviour, cognition and movement. Memory loss is not always present early. Dementia steadily worsens over time and the condition is diagnosed when cognitive decline interferes with normal daily functioning.

A core feature is REM sleep behavior disorder (RBD), in which individuals lose normal muscle paralysis during REM sleep, and act out their dreams. RBD may appear years or decades before other symptoms. Other frequent symptoms include visual hallucinations; marked fluctuations in attention or alertness; and slowness of movement, trouble walking, or rigidity. The autonomic nervous system is usually affected, resulting in changes in blood pressure, heart and gastrointestinal function, with constipation as a common symptom. Mood changes such as depression and apathy are common.

Frontotemporal dementia (frontotemporal lobar degeneration) is an umbrella term for a diverse group of uncommon disorders that primarily affect the frontal and temporal lobes of the brain—the areas generally associated with personality, behaviour and language. In frontotemporal dementia, portions of these lobes shrink (atrophy). Signs and symptoms vary, depending upon the portion of the brain affected. The frontotemporal dementias (FTD) encompass six types of dementia involving the frontal or temporal lobes. They are: behavioural variant of FTD, semantic variant primary progressive aphasia, nonfluent agrammatic variant primary progressive aphasia, corticobasal syndrome, progressive supranuclear palsy, and FTD associated with motor neuron disease.

Some people with frontotemporal dementia undergo dramatic changes in their personality and become socially inappropriate, impulsive or emotionally indifferent, while others lose the ability to use language.

In a preferred embodiment, the D4 (and 5-HT2A) receptor antagonist, reverse agonist, or partial agonist as used herein is pipamperone and the cognitive disorder as used herein is subjective cognitive decline.

In a preferred embodiment, the D4 (and 5-HT2A) receptor antagonist, reverse agonist, or partial agonist as used herein is pipamperone and the cognitive disorder as used herein is major neurocognitive disorder, preferably (caused by) Alzheimer's Disease, vascular dementia, dementia with Lewy bodies, or dementia due to Pick Disease (frontotemporal dementia).

In a preferred embodiment, the D4 (and 5-HT2A) receptor antagonist, reverse agonist, or partial agonist as used herein is pipamperone and the cognitive disorder as used herein is (dementia caused by) Alzheimer's Disease, vascular dementia, dementia with Lewy bodies, or dementia due to Pick Disease (frontotemporal dementia).

In a preferred embodiment, the D4 (and 5-HT2A) receptor antagonist, reverse agonist, or partial agonist as used herein is pipamperone and the cognitive disorder as used herein is major neurocognitive disorder, preferably (caused by) Alzheimer's Disease.

In a preferred embodiment, the D4 (and 5-HT2A) receptor antagonist, reverse agonist, or partial agonist as used herein is pipamperone and the cognitive disorder as used herein is (dementia caused by) Alzheimer's Disease.

In certain embodiments, the subject to be treated according to the invention has subjective cognitive decline (SCD).

In certain embodiments, the cognitive disorder as described herein elsewhere is subjective cognitive decline (SCD).

Subjective cognitive decline (SCD), also known as subjective cognitive impairment or subjective cognitive concerns, is characterized by a person's subjective experience of worsening in cognition (in particular in, though not limited to, the memory domain), in the absence of any objective cognitive deficits such as with detection through formal neuropsychological testing (e.g. through MMSE, where a score of at least 26, such as at least 27, at least 28, at least 29, or 30 may be obtained). SCD can also be considered as pre-mild cognitive impairment (pre-MCI) in the absence of any objective cognitive deficits such as with detection through formal neuropsychological testing or in cases where deterioration in cognitive performance cannot be detected objectively through formal neuropsychological testing. The rationale and meaning of the term of subjective cognitive decline are the following (Jenssen et al. (2014), Alzheimers Dement, 10(6):844-52). “Subjective” refers to the self-perception or self-experience of cognitive performance. It is conceptually independent of performance on a cognitive test. No “validation” of the subjective experience of cognitive capability by means of cognitive testing is required. In the context of SCD in preclinical AD, cognitive testing is required to establish a normal objective performance level, which defines preclinical AD. If SCD is studied in other conditions than preclinical AD, the respective criteria set for these conditions (e.g. MCI) need to be applied. “Cognitive” refers to any cognitive domain. It includes, but is not restricted to memory. Cognitive as opposed to memory was chosen for the following reasons: (1) the first symptoms of for instance AD are not limited to memory decline and (2) lay people may report memory decline when they actually experience decline in other cognitive domains such as executive function and vice versa (e.g., reporting a “speech problem” when the difficulty is really memory retrieval). This notwithstanding, SCD is sometimes also referred to as subjective memory impairment, complaint, or disorder. “Decline” refers to a subjectively experienced worsening of cognitive capacities. It reflects the progressive nature of cognitive deterioration, such as for instance in AD. It refers to the idea of progressive deterioration or a change from the previous level of functioning and not just an isolated complaint. The criteria for SCD can be (Net and Nitrini (2016), Dement Neuropsychol, 10(3):170-77): (1) self-experienced persistent decline in cognitive abilities compared with previously normal status and not related to an acute event; and (2) normal performance on standardized cognitive tests (for age, gender and education); while exclusion criteria are: (1) mild cognitive impairment or dementia diagnosis; and (2) decline explained by psychiatric disorders, neurological diseases (except (pre-clinical) Alzheimer's disease), other medical disorders, medication or other substance use. Additional criteria can be onset of decline within the last five years, age at onset above 60 years, associated concerns about decline and confirmation by an informant (such as a relative). Epidemiological data have shown that individuals with subjective cognitive decline are at increased risk of progression to (AD) dementia. SCD may or may not be associated with the prevalence of positive biomarkers for instance for amyloidosis (neuropathologic β-amyloid plaque and tau accumulation) and neurodegeneration. Furthermore, individuals with subjective cognitive decline in combination with major depressive disorder even have a higher increased risk of progression to (AD) dementia (Liew (2019), Alzheimers Res Ther, 11(1):70; doi 10.1186/s13195-019-0527-7).

In certain embodiments, the subject to be treated according to the invention has mild cognitive impairment (MCI) or pre-mild cognitive impairment (pre-MCI).

Mild cognitive impairment (MCI), also known as incipient dementia and isolated memory impairment, is the stage between the expected cognitive decline of normal aging and the more serious decline of dementia. It is a neurological disorder that occurs in older adults which involves cognitive impairments with minimal impairment in instrumental activities of daily living. It can involve problems with memory, language, thinking and judgment that are greater than normal age-related changes. When having mild cognitive impairment, a subject may be aware that his memory or mental function has “slipped.” Family and close friends also may notice a change. But these changes aren't severe enough to significantly interfere with daily life and usual activities. Mild cognitive impairment may increase the risk of later developing dementia caused by Alzheimer's disease or other neurological conditions. But some people with mild cognitive impairment never get worse, and a few eventually get better. There's no single cause of mild cognitive impairment (MCI), just as there's no single outcome for the disorder. Symptoms of MCI may remain stable for years, progress to Alzheimer's disease or another type of dementia, or improve overtime. Current evidence indicates that MCI often, but not always, develops from a lesser degree of the same types of brain changes seen in Alzheimer's disease or other forms of dementia. Some of these changes have been identified in autopsy studies of people with MCI. These changes include: abnormal clumps of beta-amyloid protein (plaques) and microscopic protein clumps of tau characteristic of Alzheimer's disease (tangles); Lewy bodies, which are microscopic clumps of another protein associated with Parkinson's disease, dementia with Lewy bodies and some cases of Alzheimer's disease; Small strokes or reduced blood flow through brain blood vessels. Pre-MCI can be considered as an intermediate stage between no cognitive impairment (NCI) and mild cognitive impairment (MCI), and can be characterized by cognitive, functional, motor, behavioural and/or imaging features that are intermediate between NCI and MCI states. Pre-MCI can be SCD in the absence of any objective cognitive deficits such as with detection through formal neuropsychological testing or if deterioration in cognitive performance cannot be detected objectively through formal neuropsychological testing (e.g. only imaging or biomarkers reveal impairment).

In certain embodiments, the subject to be treated according to the invention has a prodromal neurodegenerative disease, preferably prodromal Alzheimer's disease, or a pre-prodromal neurodegenerative disease, preferably pre-prodromal Alzheimer's disease.

A prodrome is an early sign or symptom (or set of signs and symptoms), which often indicate the onset of a disease before more diagnostically specific signs and symptoms develop. The prodrome is a period during which an individual experiences some symptoms and/or a change in functioning, which can signal the impending onset of a disease or disorder. It is otherwise known as the prodromal phase when referring to the subsyndromal stage or the early abnormalities, for instance in behaviour, mood, and/or cognition before illness onset. In the very early stages, for instance in the case of neurodegenerative disorders such as Alzheimer's disease, when the neuropathology is confined, the neuropsychological symptoms may not be apparent at all (latent phase), but as the pathology cumulates overtime early symptoms emerge (prodrome stage) followed later by fully manifest clinical disease (dementia stage). Prodromal AD can be defined as the symptomatic predementia phase of AD, generally included in the MCI category; this stage is characterised by symptoms not severe enough to meet currently accepted diagnostic criteria for AD. Typically, prodromal Alzheimer's disease is the very early form of Alzheimer's when memory is deteriorating but a person remains functionally independent. MCI can be considered a (symptom of) prodromal neurodegenerative disease such as pre-prodromal Alzheimer's disease, in particular if positive for the relevant biomarkers, such as in the case of Alzheimer's disease amyloidosis or neurodegeneration, and where deterioration in cognitive performance can detected objectively through formal neuropsychological testing, but not severe enough to be classified as dementia. Subjectively perceived memory problems (such as SCD) can be an indicator of a pre-prodromal stage of a neurodegenerative disease such as Alzheimer's disease. SCD can be considered (a symptom of) a pre-prodromal neurodegenerative disease such as pre-prodromal Alzheimer's disease, in particular if positive for the relevant biomarkers, such as in the case of Alzheimer's disease amyloidosis (neuropathologic β-amyloid plaque and tau accumulation) or neurodegeneration, but in the absence of any objective cognitive deficits such as with detection through formal neuropsychological testing or where deterioration in cognitive performance cannot detected objectively through formal neuropsychological testing.

In certain embodiments, the subject to be treated according to the invention has a pre-clinical or asymptomatic neurodegenerative disease, preferably pre-clinical or asymptomatic Alzheimer's disease.

Subjects having a pre-clinical or asymptomatic neurodegenerative disease, such as Alzheimer's disease, are characterized by the presence of biomarkers for the disease, without being impaired by the disease of without showing the characteristic symptoms of the disease. In particular, individuals with preclinical or asymptomatic AD have AD pathology without clinical symptoms yet. Accordingly, preclinical or asymptomatic AD is characterised by positive biomarkers, such as amyloidosis (neuropathologic β-amyloid plaque and tau accumulation) and neurodegeneration, without cognitive impairment or in the absence of any objective cognitive deficits such as with detection through formal neuropsychological testing or if deterioration in cognitive performance cannot detected objectively through formal neuropsychological testing, such as for instance in subjects with SCD, pre-MCI, and/or pre-prodromal AD. Vice versa, subjects with SCD, pre-MCI, and/or pre-prodromal AD may be characterized as having preclinical or asymptomatic AD.

As used herein, “transcranial direct current stimulation” or tDCS refers to a non-invasive form of neurostimulation that uses constant, low direct current delivered via electrodes on the head (in contrast to cranial electrotherapy stimulation, which generally uses alternating current the same way). A fixed current between 1 and 2 mA is typically applied. tDCS works by applying a positive (anodal) or negative (cathodal) current via electrodes to an area, facilitating the depolarization or hyperpolarization of neurons, respectively. The positioning of the anode and cathode electrodes is used to influence how current flows, and where in the brain it does. The current delivered by tDCS is not considered strong enough to trigger an action potential in a neuron; its sub-threshold effect works by bringing the neurons closer to, or farther from firing. Plainly, tDCS augments the resting voltage of the neuronal membrane to prod a neuron's activity in a desired direction. In this way, tDCS may work by strengthening or weakening synaptic transmission between neurons by augmenting synaptic plasticity which is, in turn, the cellular basis of learning. Accordingly, one of the aspects of tDCS is its ability to achieve cortical changes even after the stimulation is ended. The duration of this change depends on the length of stimulation as well as the intensity of stimulation. The effects of stimulation increase as the duration of stimulation increases or the strength of the current increases. The way that the stimulation changes brain function is either by causing the neuron's resting membrane potential to depolarize or hyperpolarize. When positive stimulation (anodal tDCS) is delivered, the current causes a depolarization of the resting membrane potential, which increases neuronal excitability and allows for more spontaneous cell firing. When negative stimulation (cathodal tDCS) is delivered, the current causes a hyperpolarization of the resting membrane potential. This decreases neuron excitability due to the decreased spontaneous cell firing.

To set up the tDCS device, the electrodes and the skin need to be prepared. This ensures a low resistance connection between the skin and the electrode. The careful placement of the electrodes may be crucial to successful tDCS technique. The electrode pads come in various sizes with benefits to each size. A smaller sized electrode achieves a more focused stimulation of a site while a larger electrode ensures that the entirety of the region of interest is being stimulated. One of the electrodes is placed over the region of interest and the other electrode, the reference electrode, is placed in another location in order to complete the circuit. This reference electrode is usually placed on the neck or shoulder of the opposite side of the body than the region of interest. Since the region of interest may be small, it is often useful to locate this region before placing the electrode by using a brain imaging technique such as fMRI or PET. Once the electrodes are placed correctly, the stimulation can be started. Many devices have a built-in capability that allows the current to be “ramped up” or increased gradually until the necessary current is reached. This decreases the amount of stimulation effects felt by the person receiving the tDCS. After the stimulation has been started, the current will continue for the amount of time set on the device and then will automatically be shut off.

In certain embodiments, the tDCS is anodal tDSC. In certain embodiments, the tDCS is cathodal tDCS. Preferably, the tDCS is anodal tDCS.

In certain embodiments, a current ranging from 0.5 to 2.5 mA is applied during tDCS, preferably ranging from 1 to 2 mA, such as 1.0 to 2.0 mA, more preferably ranging from 1.5 to 2 mA, such as 1.5 to 2.0 mA. In certain embodiments, the current is applied on a surface (or the scalp) ranging from 10 to 50 cm², preferably ranging from 20 to 40 cm², such as about 30 cm² or 30 cm². In certain embodiments, a current ranging from 0.015 to 0.075 mA/cm² is applied during tDCS, preferably ranging from 0.03 to 0.06 mA/cm², more preferably ranging from 0.045 to 0.06 mA/cm².

In certain embodiments, tDCS is applied continuously for a time ranging from 5 minutes to 1 hour, preferably ranging from 10 minutes to 45 minutes, more preferably ranging from 20 minutes to 40 minutes, such as ranging from 25 minutes to 35 minutes, such as 30 minutes. In certain embodiments, tDCS is applied continuously for about 30 minutes or for 30 minutes. It will be appreciated that such continuous application of tDCS constitutes a single tDCS session.

In certain embodiments, multiple sessions of tDCS have been, are, or will be applied, or a subject has been, is, or will be subjected to multiple sessions. In certain embodiments, multiple sessions are at least 2 sessions, preferably at least 5 sessions, more preferably at least 10 sessions, such as at least 15 sessions or 15 sessions. In certain embodiments, multiple sessions are at most 50 sessions, preferably at most 40 sessions, such as 15 sessions. In certain embodiments, multiple sessions are from 5 to 50 sessions, preferably from 5 to 40 sessions, more preferably from 10 to 40 sessions, such as from 10 to 30 sessions or from 10 to 20 sessions. In certain embodiments, the multiple sessions are daily consecutive sessions. In certain embodiments, the multiple sessions are bidaily consecutive sessions. In certain embodiments, the multiple sessions are on non-consecutive days. In certain embodiments, the multiple sessions are every other day. In certain embodiments, the multiple sessions are every three days. In certain embodiments, the multiple sessions are from 1 to 7 sessions per week. In certain embodiments, the multiple sessions are from 2 to 7 sessions per week. In certain embodiments, the multiple sessions are from 3 to 7 sessions per week. In certain embodiments, the multiple sessions are from 4 to 7 sessions per week. In certain embodiments, the multiple sessions are from 5 to 7 sessions per week. In certain embodiments, the multiple sessions are from 6 to 7 sessions per week. In certain embodiments, the multiple sessions are 2 sessions per week. In certain embodiments, the multiple sessions are 3 sessions per week. In certain embodiments, the multiple sessions are 4 sessions per week. In certain embodiments, the multiple sessions are 5 sessions per week. In certain embodiments, the multiple sessions are 6 sessions per week. In certain embodiments, the multiple sessions are 7 sessions per week. In certain embodiments, the multiple sessions are multiple sessions as defined above for at least 1 week or 1 week, preferably at least 2 weeks or 2 weeks, more preferable at least 3 weeks or 3 weeks. In certain embodiments, the multiple sessions are multiple sessions as defined above for at most 12 weeks or 12 weeks, preferably at most 10 weeks or 10 weeks, more preferable at most 8 weeks or 8 weeks. In certain embodiments, the multiple sessions are multiple sessions as defined above for at least 1 week or 1 week and at most 12 weeks or 12 weeks, preferably at least 2 weeks or 2 weeks and at most 10 weeks or 10 weeks, more preferable at least 3 weeks or 3 weeks and at most 8 weeks or 8 weeks. In will be understood that the sessions may or may not be on consecutive days. It will be further understood that the multiple sessions may or may not be equally spread over the entire period. For example and without limitation, the multiple sessions may be every other day for the entire period or the multiple sessions may be daily or every other day for a certain period followed by one or several days without a session and then again followed by sessions every day or every other day (e.g. daily sessions during week days only, and not during weekend days).

In certain embodiments, a subject has been, is, or will be subjected to tDCS at least 10 times, preferably at least 15 times or 15 times, for between 15 and 45 minutes per session, preferably about 30 minutes or 30 minutes, per three weeks. Preferably, the amount of sessions per three weeks is at most 40, more preferably at most 30, even more preferably at most 25 or 20. It will be understood that the total treatment period may last for at least three weeks, such as 3 weeks, 4 weeks, 5 weeks, 6 weeks, 7 weeks, 8 weeks, or more, preferably at most 12 weeks, more preferably at most 10 weeks.

In certain embodiments, a subject has been, is, or will be subjected to anodal tDCS at least 10 times, preferably at least 15 times or 15 times, for between 15 and 45 minutes per session, preferably about 30 minutes or 30 minutes, per three weeks. Preferably, the amount of sessions per three weeks is at most 40, more preferably at most 30, even more preferably at most 25 or 20. It will be understood that the total treatment period may last for at least three weeks, such as 3 weeks, 4 weeks, 5 weeks, 6 weeks, 7 weeks, 8 weeks, or more, preferably at most 12 weeks, more preferably at most 10 weeks.

In certain embodiments, a subject has been, is, or will be subjected to cathodal tDCS at least 10 times, preferably at least 15 times or 15 times, for between 15 and 45 minutes per session, preferably about 30 minutes or 30 minutes, per three weeks. Preferably, the amount of sessions per three weeks is at most 40, more preferably at most 30, even more preferably at most 25 or 20. It will be understood that the total treatment period may last for at least three weeks, such as 3 weeks, 4 weeks, 5 weeks, 6 weeks, 7 weeks, 8 weeks, or more, preferably at most 12 weeks, more preferably at most 10 weeks.

In certain embodiments, a subject has been, is, or will be subjected to tDCS at least 10 times, preferably at least 15 times or 15 times, for between 15 and 45 minutes per session, preferably about 30 minutes or 30 minutes, per three weeks. Preferably, the amount of sessions per three weeks is at most 40, more preferably at most 30, even more preferably at most 25 or 20. It will be understood that the total treatment period may last for at least three weeks, such as 3 weeks, 4 weeks, 5 weeks, 6 weeks, 7 weeks, 8 weeks, or more, preferably at most 12 weeks, more preferably at most 10 weeks; wherein a current ranging from 0.5 to 2.5 mA is applied during tDCS, preferably ranging from 1 to 2 mA, such as 1.0 to 2.0 mA, more preferably ranging from 1.5 to 2 mA, such as 1.5 to 2.0 mA or about 2 or 2.0 mA or 2 or 2.0 mA, preferably per 30 cm².

In certain embodiments, a subject has been, is, or will be subjected to anodal tDCS at least 10 times, preferably at least 15 times or 15 times, for between 15 and 45 minutes per session, preferably about 30 minutes or 30 minutes, per three weeks. Preferably, the amount of sessions per three weeks is at most 40, more preferably at most 30, even more preferably at most 25 or 20. It will be understood that the total treatment period may last for at least three weeks, such as 3 weeks, 4 weeks, 5 weeks, 6 weeks, 7 weeks, 8 weeks, or more, preferably at most 12 weeks, more preferably at most 10 weeks; wherein a current ranging from 0.5 to 2.5 mA is applied during tDCS, preferably ranging from 1 to 2 mA, such as 1.0 to 2.0 mA, more preferably ranging from 1.5 to 2 mA, such as 1.5 to 2.0 mA or about 2 or 2.0 mA or 2 or 2.0 mA, preferably per 30 cm².

In certain embodiments, a subject has been, is, or will be subjected to cathodal tDCS at least 10 times, preferably at least 15 times or 15 times, for between 15 and 45 minutes per session, preferably about 30 minutes or 30 minutes, per three weeks. Preferably, the amount of sessions per three weeks is at most 40, more preferably at most 30, even more preferably at most 25 or 20. It will be understood that the total treatment period may last for at least three weeks, such as 3 weeks, 4 weeks, 5 weeks, 6 weeks, 7 weeks, 8 weeks, or more, preferably at most 12 weeks, more preferably at most 10 weeks; wherein a current ranging from 0.5 to 2.5 mA is applied during tDCS, preferably ranging from 1 to 2 mA, such as 1.0 to 2.0 mA, more preferably ranging from 1.5 to 2 mA, such as 1.5 to 2.0 mA or about 2 or 2.0 mA or 2 or 2.0 mA, preferably per 30 cm².

In certain embodiments, a subject has been, is, or will be subjected to anodal tDCS at least 15 times and at most 25 times per three weeks, for between 15 and 45 minutes per session; wherein the total treatment period ranges from 3 to 12 weeks; and wherein a current ranging from 1.8 to 2.2 is applied, preferably per 30 cm².

In certain embodiments, a subject has been, is, or will be subjected to cathodal tDCS at least 15 times and at most 25 times per three weeks, for between 15 and 45 minutes per session; wherein the total treatment period ranges from 3 to 12 weeks; and wherein a current ranging from 1.8 to 2.2 is applied, preferably per 30 cm².

In certain preferred embodiments, tDCS is anodal tDCS.

The skilled person will understand that the brain region to be subjected to tDCS (to a certain extent) may depend on the desired outcome and/or the type of cognitive disorder. Etiology and pathophysiological conditions or diverse cognitive disorders are well known in the art. The skilled person based hereon can design tDCS and in particular the relevant brain regions to be treated accordingly. Similarly, depending on the cognitive function to be improved, which are well known in the art to reside or be governed by particular brain regions, the skilled person based hereon can design tDCS and in particular the relevant brain regions to be treated accordingly.

It will be understood that as tDCS is non-invasive, tDCS is essentially applied to the (cerebral) cortex. In certain embodiments, tDCS is applied to the parietal lobe. In certain embodiments, tDCS is applied to the temporal lobe. In certain embodiments, tDCS is applied to the frontal lobe. In certain embodiments, tDCS is applied to the occipital lobe. In certain embodiments, tDCS is applied to the left parietal lobe. In certain embodiments, tDCS is applied to the left temporal lobe. In certain embodiments, tDCS is applied to the left frontal lobe. In certain embodiments, tDCS is applied to the left occipital lobe. In certain embodiments, tDCS is applied to the right parietal lobe. In certain embodiments, tDCS is applied to the right temporal lobe. In certain embodiments, tDCS is applied to the right frontal lobe. In certain embodiments, tDCS is applied to the right occipital lobe. In certain embodiments, tDCS is applied to the left and right parietal lobe (i.e. bilateral). In certain embodiments, tDCS is applied to the left and right temporal lobe. In certain embodiments, tDCS is applied to the left and right frontal lobe. In certain embodiments, tDCS is applied to the left and right occipital lobe. In certain preferred embodiments, the anode is placed over the left lobe (or hemisphere or subsection) and the cathode is placed over the right lobe (or hemisphere or subsection). In certain embodiments, the anode is placed over the right lobe (or hemisphere or subsection) and the cathode is placed over the left lobe (or hemisphere or subsection). The skilled person will understand that tDCS can also be applied to certain subsections of the respective lobes or to multiple lobes or boundary regions of different lobes, such as temperoparietal or frontotemporal or frontoparietal regions. It will also be understood that different regions may be subjected to tDCS on the left and right hemisphere. In certain preferred embodiments, tDCS is applied to the prefrontal cortex. In certain embodiments, tDCS is applied to left prefrontal cortex. In certain embodiments, tDCS is applied to the right prefrontal cortex. In certain embodiments, tDCS is applied to the left and right prefrontal cortex. In certain preferred embodiments, tDCS is applied to the dorsolateral prefrontal cortex (DLPFC). In certain embodiments, tDCS is applied to the left DLPFC. In certain embodiments, tDCS is applied to the right DLPFC. In certain embodiments, tDCS is applied to the left and right DLPFC. Preferably, tDCS is applied bilaterally. Preferably, the anode is placed over the left prefrontal cortex, such as preferably the left DLPFC, and the cathode is placed over the right prefrontal cortex, such as preferably the right DLPFC. The excitatory stimulation (anode) may be delivered to the left dorsolateral prefrontal cortex in order to increase neuronal activation in the area. The inhibitory stimulation (cathode) may be delivered to the right dorsolateral prefrontal cortex with the aim of suppressing hyperactivity in the area.

In certain embodiments, a subject has been, is, or will be subjected to tDCS of the left and/or right DLPFC at least 10 times, preferably at least 15 times or 15 times, for between 15 and 45 minutes per session, preferably about 30 minutes or 30 minutes, per three weeks. Preferably, the amount of sessions per three weeks is at most 40, more preferably at most 30, even more preferably at most 25 or 20. It will be understood that the total treatment period may last for at least three weeks, such as 3 weeks, 4 weeks, 5 weeks, 6 weeks, 7 weeks, 8 weeks, or more, preferably at most 12 weeks, more preferably at most 10 weeks.

In certain embodiments, a subject has been, is, or will be subjected to anodal tDCS of the left and/or right DLPFC at least 10 times, preferably at least 15 times or 15 times, for between 15 and 45 minutes per session, preferably about 30 minutes or 30 minutes, per three weeks. Preferably, the amount of sessions per three weeks is at most 40, more preferably at most 30, even more preferably at most 25 or 20. It will be understood that the total treatment period may last for at least three weeks, such as 3 weeks, 4 weeks, 5 weeks, 6 weeks, 7 weeks, 8 weeks, or more, preferably at most 12 weeks, more preferably at most 10 weeks.

In certain embodiments, a subject has been, is, or will be subjected to cathodal tDCS of the left and/or right DLPFC at least 10 times, preferably at least 15 times or 15 times, for between 15 and 45 minutes per session, preferably about 30 minutes or 30 minutes, per three weeks. Preferably, the amount of sessions per three weeks is at most 40, more preferably at most 30, even more preferably at most 25 or 20. It will be understood that the total treatment period may last for at least three weeks, such as 3 weeks, 4 weeks, 5 weeks, 6 weeks, 7 weeks, 8 weeks, or more, preferably at most 12 weeks, more preferably at most 10 weeks.

In certain embodiments, a subject has been, is, or will be subjected to tDCS of the left and/or right DLPFC at least 10 times, preferably at least 15 times or 15 times, for between 15 and 45 minutes per session, preferably about 30 minutes or 30 minutes, per three weeks. Preferably, the amount of sessions per three weeks is at most 40, more preferably at most 30, even more preferably at most 25 or 20. It will be understood that the total treatment period may last for at least three weeks, such as 3 weeks, 4 weeks, 5 weeks, 6 weeks, 7 weeks, 8 weeks, or more, preferably at most 12 weeks, more preferably at most 10 weeks; wherein a current ranging from 0.5 to 2.5 mA is applied during tDCS, preferably ranging from 1 to 2 mA, such as 1.0 to 2.0 mA, more preferably ranging from 1.5 to 2 mA, such as 1.5 to 2.0 mA or about 2 or 2.0 mA or 2 or 2.0 mA, preferably per 30 cm².

In certain embodiments, a subject has been, is, or will be subjected to anodal tDCS of the left and/or right DLPFC at least 10 times, preferably at least 15 times or 15 times, for between 15 and 45 minutes per session, preferably about 30 minutes or 30 minutes, per three weeks. Preferably, the amount of sessions per three weeks is at most 40, more preferably at most 30, even more preferably at most 25 or 20. It will be understood that the total treatment period may last for at least three weeks, such as 3 weeks, 4 weeks, 5 weeks, 6 weeks, 7 weeks, 8 weeks, or more, preferably at most 12 weeks, more preferably at most 10 weeks; wherein a current ranging from 0.5 to 2.5 mA is applied during tDCS, preferably ranging from 1 to 2 mA, such as 1.0 to 2.0 mA, more preferably ranging from 1.5 to 2 mA, such as 1.5 to 2.0 mA or about 2 or 2.0 mA or 2 or 2.0 mA, preferably per 30 cm².

In certain embodiments, a subject has been, is, or will be subjected to cathodal tDCS of the left and/or right DLPFC at least 10 times, preferably at least 15 times or 15 times, for between 15 and 45 minutes per session, preferably about 30 minutes or 30 minutes, per three weeks. Preferably, the amount of sessions per three weeks is at most 40, more preferably at most 30, even more preferably at most 25 or 20. It will be understood that the total treatment period may last for at least three weeks, such as 3 weeks, 4 weeks, 5 weeks, 6 weeks, 7 weeks, 8 weeks, or more, preferably at most 12 weeks, more preferably at most 10 weeks; wherein a current ranging from 0.5 to 2.5 mA is applied during tDCS, preferably ranging from 1 to 2 mA, such as 1.0 to 2.0 mA, more preferably ranging from 1.5 to 2 mA, such as 1.5 to 2.0 mA or about 2 or 2.0 mA or 2 or 2.0 mA, preferably per 30 cm².

In certain embodiments, a subject has been, is, or will be subjected to anodal tDCS of the left and/or right DLPFC at least 15 times and at most 25 times per three weeks, for between 15 and 45 minutes per session; wherein the total treatment period ranges from 3 to 12 weeks; and wherein a current ranging from 1.8 to 2.2 is applied, preferably per 30 cm².

In certain embodiments, a subject has been, is, or will be subjected to cathodal tDCS of the left and/or right DLPFC at least 15 times and at most 25 times per three weeks, for between 15 and 45 minutes per session; wherein the total treatment period ranges from 3 to 12 weeks; and wherein a current ranging from 1.8 to 2.2 is applied, preferably per 30 cm².

The compounds, methods, and uses according to the invention as described herein are suitable for preventing, delaying or postponing the onset and/or delaying, postponing, or preventing progression, and/or treating a cognitive disorder, and/or maintaining or improving cognitive function in a subject and/or for preventing suicide or preventing or reducing suicidal thoughts, and/or for preventing, delaying or postponing the onset and/or delaying, postponing, or preventing progression, and/or treating an affective disorder, preferably (mild) major depressive disorder.

As used herein, the terms “treatment”, “treating”, and the like relate to amelioration or elimination of a developed cognitive disorder once it has been established or alleviation of the characteristic symptoms of such disorder. It is to be understood that compete elimination of the disorder need not be achieved for the term treatment to apply. Partial treatment is hence also encompassed by the term treatment. Also a future recurrence of the cognitive disorder does not preclude the term treatment to apply if at one point at least the disease is eliminated or ameliorated.

As used herein the terms “preventing”, “prevention”, and the like relate to preventing the onset of a cognitive disorder or of symptoms associated with a disorder, including partial prevention by reducing the severity of the disorder or symptoms associated therewith by treatment prior to affliction with said disease or condition. Such prevention or reduction prior to affliction refers to administration of the compound or composition or applying the methods of the invention to a patient that is not at the time of administration afflicted with the cognitive disorder. “Preventing” also encompasses (partially) preventing the recurrence or relapse-prevention of a cognitive disorder or of symptoms associated therewith, for instance after a period of improvement.

As used herein the terms “delaying the onset” and the like relate to postponement of the onset of a cognitive disorder or of symptoms associated with the disorder. The onset of the disorder may for instance be postponed by at least half a year, such as at least one year. The exact time of the onset of the disease may possibly not be unequivocally determined. However, the time of diagnosis of the cognitive disorder may serve as a proxy.

As used herein the terms “delaying the progression” or “preventing the progression” and the like relate to delaying or preventing the cognitive disorder (once it has been established) or the characteristic symptoms of such disorder from getting more severe over time. It will be understood that delay or prevention may not need to be indefinite. It will be further understood that such delay or prevention need not be complete, but may also be partial.

It should be clear that cognitive disorders may be responsible for physical complaints. In this respect, the terms specified above also include prevention, delaying the onset and/or progression, or treatment of a physical disease or condition or amelioration or elimination of the developed physical disease, characteristic, or condition once it has been established or alleviation of the characteristic symptoms of such conditions.

As used herein, the term “medicament” also encompasses the terms “drug”, “therapeutic”, “potion” or other terms which are used in the field of medicine to indicate a preparation with therapeutic or prophylactic effect.

In certain preferred embodiments, the compounds, methods, and uses according to the invention as described herein prevent or delay or postpone the onset or progression of cognitive disorders, and/or maintain or improve cognitive function in a subject, as for instance evidenced by or accompanied by an improvement of MMSE, a reduction of clinical restlessness events, and/or an increase in gamma power.

In certain embodiments, the D4 (and 5-HT2a) receptor antagonist, reverse agonist, or partial agonist can be or is to be administered simultaneously with (but separate from) or sequentially to tDCS, in either order. As indicated herein, simultaneous administration of the D4 (and 5-HT2a) receptor antagonist, reverse agonist, or partial agonist and application of tDCS does not require both to happen at the exact same time. Rather, “simultaneous” refers to a particular time frame in which both the D4 (and 5-HT2a) receptor antagonist, reverse agonist, or partial agonist is administered (preferably according to the required treatment regime as described herein elsewhere) and tDCS is performed (preferably for the required number of sessions and duration of the sessions over the required time period as described herein elsewhere). In contrast, “sequentially” refers to non-overlapping time frames during which the D4 (and 5-HT2a) receptor antagonist, reverse agonist, or partial agonist is or is to be administered and tDCS is applied.

In certain embodiments, the D4 (and 5-HT2a) receptor antagonist, reverse agonist, or partial agonist is or is to be administered to a subject undergoing tDCS, i.e. the D4 (and 5-HT2a) receptor antagonist, reverse agonist, or partial agonist is administered simultaneously with tDCS.

In certain embodiments, the D4 (and 5-HT2a) receptor antagonist, reverse agonist, or partial agonist is or is to be administered to a subject having undergone tDCS, i.e. the D4 (and 5-HT2a) receptor antagonist, reverse agonist, or partial agonist is administered sequentially to and after tDCS.

In certain embodiments, the D4 (and 5-HT2a) receptor antagonist, reverse agonist, or partial agonist is or is to be administered to a subject that will undergo tDCS, i.e. the D4 (and 5-HT2a) receptor antagonist, reverse agonist, or partial agonist is administered sequentially to and prior to tDCS.

In certain embodiments administration of the D4 (and 5-HT2a) receptor antagonist, reverse agonist, or partial agonist and tDCS are adjunctive prophylactic or therapeutic treatments.

In certain embodiments, the D4 (and 5-HT2a) receptor antagonist, reverse agonist, or partial agonist is or is to be administered simultaneously to applying tDCS for a first period of time followed by sequential administration (optionally for a second time period or indefinitely) of the D4 (and 5-HT2a) receptor antagonist, reverse agonist, or partial agonist without applying tDCS. In certain embodiments, the D4 (and 5-HT2a) receptor antagonist, reverse agonist, or partial agonist is or is to be administered as an adjunctive prophylactic or therapeutic treatment to tDCS for a first time period, followed by discontinuation of tDCS and maintaining administration of the D4 (and 5-HT2a) receptor antagonist, reverse agonist, or partial agonist.

If the D4 (and 5-HT2a) receptor antagonist, reverse agonist, or partial agonist and tDCS are administered/applied sequentially the time between administration of the D4 (and 5-HT2a) receptor antagonist, reverse agonist, or partial agonist and application of tDCS is typically short, such as preferably at most 1 week, more preferably at most 4 days, even more preferably at most 2 days, most preferably at most 1 day or 1 day. This is also applicable in the case where, the D4 (and 5-HT2a) receptor antagonist, reverse agonist, or partial agonist is or is to be administered simultaneously to applying tDCS for a first period of time followed by sequential administration of the D4 (and 5-HT2a) receptor antagonist, reverse agonist, or partial agonist without applying tDCS. In such case, administration of the D4 (and 5-HT2a) receptor antagonist, reverse agonist, or partial agonist may just be continued, while tDCS is discontinued.

In certain embodiments, the subject to be treated according to the invention with the D4 (and 5-HT2a) receptor antagonist, reverse agonist, or partial agonist is characterised by previously having been treated (prior to treatment with the D4 (and 5-HT2a) receptor antagonist, reverse agonist, or partial agonist) with tDCS, preferably for a period ranging from 1 to 12 weeks, more preferably 2 to 10 weeks, such as 3 to 8 weeks, and preferably according to the required treatment regime as described herein elsewhere.

In certain embodiments, the subject to be treated according to the invention with the D4 (and 5-HT2a) receptor antagonist, reverse agonist, or partial agonist is characterised by previously having been (simultaneously) treated (prior to treatment with the D4 (and 5-HT2a) receptor antagonist, reverse agonist, or partial agonist) with a D4 (and 5-HT2a) receptor antagonist, reverse agonist, or partial agonist (which may be the same or a different D4 (and 5-HT2a) receptor antagonist, reverse agonist, or partial agonist) and tDCS, preferably for a period ranging from 1 to 12 weeks, more preferably 2 to 10 weeks, such as 3 to 8 weeks, and preferably according to the required treatment regime as described herein elsewhere.

In certain embodiments, the subject to be treated according to the invention with the D4 (and 5-HT2a) receptor antagonist, reverse agonist, or partial agonist is characterised by previously having been treated (prior to treatment with the D4 (and 5-HT2a) receptor antagonist, reverse agonist, or partial agonist) with tDCS, preferably for a period until the subject exhibited an MMSE score increase of at least 1 point and/or a decrease in qualified clinical restlessness events during sleep of at least 25%, preferably at least 50%.

In certain embodiments, the subject to be treated according to the invention with the D4 (and 5-HT2a) receptor antagonist, reverse agonist, or partial agonist is characterised by previously having been (simultaneously) treated (prior to treatment with the D4 (and 5-HT2a) receptor antagonist, reverse agonist, or partial agonist) with a D4 (and 5-HT2a) receptor antagonist, reverse agonist, or partial agonist (which may be the same or a different D4 (and 5-HT2a) receptor antagonist, reverse agonist, or partial agonist) and tDCS, preferably for a period until the subject exhibited an MMSE score increase of at least 1 point and/or a decrease in qualified clinical restlessness events during sleep of at least 25%, preferably at least 50%.

In certain embodiments, the subject to be treated according to the invention with the D4 (and 5-HT2a) receptor antagonist, reverse agonist, or partial agonist is characterised by previously having been treated (prior to treatment with the D4 (and 5-HT2a) receptor antagonist, reverse agonist, or partial agonist) with tDCS, preferably wherein the subject exhibited an MMSE score increase of at least 1 point and/or a decrease in qualified clinical restlessness events during sleep of at least 25%, preferably at least 50%.

In certain embodiments, the subject to be treated according to the invention with the D4 (and 5-HT2a) receptor antagonist, reverse agonist, or partial agonist is characterised by having been (simultaneously) treated (prior to treatment with the D4 (and 5-HT2a) receptor antagonist, reverse agonist, or partial agonist) with a D4 (and 5-HT2a) receptor antagonist, reverse agonist, or partial agonist (which may be the same or a different D4 (and 5-HT2a) receptor antagonist, reverse agonist, or partial agonist) and tDCS, preferably wherein the subject exhibited an MMSE score increase of at least 1 point and/or a decrease in qualified clinical restlessness events during sleep of at least 25%, preferably at least 50%.

In certain embodiments, the subject to be treated according to the invention with the D4 (and 5-HT2a) receptor antagonist, reverse agonist, or partial agonist is characterised by having been treated (prior to treatment with the D4 (and 5-HT2a) receptor antagonist, reverse agonist, or partial agonist) with tDCS, preferably fora period ranging from 1 to 12 weeks, more preferably 2 to 10 weeks, such as 3 to 8 weeks, and preferably according to the required treatment regime as described herein elsewhere; and wherein the subject exhibited an MMSE score increase of at least 1 point and/or a decrease in qualified clinical restlessness events during sleep of at least 25%, preferably at least 50%.

In certain embodiments, the subject to be treated according to the invention with the D4 (and 5-HT2a) receptor antagonist, reverse agonist, or partial agonist is characterised by having been (simultaneously) treated (prior to treatment with the D4 (and 5-HT2a) receptor antagonist, reverse agonist, or partial agonist) with a D4 (and 5-HT2a) receptor antagonist, reverse agonist, or partial agonist (which may be the same or a different D4 (and 5-HT2a) receptor antagonist, reverse agonist, or partial agonist) and tDCS, preferably for a period ranging from 1 to 12 weeks, more preferably 2 to 10 weeks, such as 3 to 8 weeks, and preferably according to the required treatment regime as described herein elsewhere; and wherein the subject exhibited an MMSE score increase of at least 1 point and/or a decrease in qualified clinical restlessness events during sleep of at least 25%, preferably at least 50%.

In certain embodiments, the D4 (and 5-HT2a) receptor antagonist, reverse agonist, or partial agonist is administered within one month after discontinuation of tDCS, preferably within 3 weeks after discontinuation of tDCS, more preferably within 2 weeks after discontinuation of tDCS, such as 2 weeks after discontinuation of tDCS or 1 week after discontinuation of tDCS.

As used herein, the term “gamma power” refers to the magnitude or amplitude of gamma brain oscillations or waves, as is known in the art. The human mental stage in which gamma waves are typically involved is higher mental activity (such as simultaneous processing of information from different brain areas and creating the unity of conscious perception), cognitive regeneration, recovery, or recuperation, and motor function. Gamma waves are also implicated during rapid eye movement sleep and anesthesia, which involves visualizations. The term gamma power can be used interchangeously with gamma power spectral. By means of further guidance, and without limitation, gamma power can be determined by spectral analysis of brain waves, as is known in the art, and which involves decomposition of EEG data into functionally distinct frequency bands, such as delta (0.5-4 Hz), theta (4-8 Hz), alpha (8-12 Hz), beta (12-30 Hz), and gamma (30-100 Hz), which is commonly achieved through Fourier transform. The power is expressed in V², typically pV². Gamma power can be expressed as the sum of all the magnitudes measured in the specific Hz band, for instance during a specified time period, such as for instance during (total) REM sleep. “Relative gamma power” is a representation of the percentage of gamma power over total brain wave power.

In a preferred embodiment, relative gamma power is determined.

Maintaining (relative) gamma power indicates that over time (relative) gamma power is not (substantially) altered, in particular, that over time (relative) gamma power is not (substantially) decreased, such as not statistically significantly decreased. Increasing (relative) gamma power indicates that over time (relative) gamma power is increased, preferably by at least 1%, such as at least 2%, at least 3%, at least 4%, or at least 5%, such as at least 10%. It will be understood that maintenance or increase in (relative) gamma power is compared between similar conditions (e.g. in both cases during REM sleep and in both cases at the frontal cortex).

In certain embodiments, (relative) gamma power is determined at the cortex, preferably the frontal cortex, more preferably the prefrontal cortex, most preferably the dorsolateral prefrontal cortex.

In certain embodiments, (relative) gamma power is determined at the left and right cortex, preferably the left and right frontal cortex, more preferably the left and right prefrontal cortex, most preferably the left and right dorsolateral prefrontal cortex.

In certain embodiments, (relative) gamma power is determined during sleep, preferably during REM sleep, more preferable during total REM sleep.

In certain embodiments, (relative) gamma power is determined at the cortex, preferably the frontal cortex, more preferably the prefrontal cortex, most preferably the dorsolateral prefrontal cortex and during sleep, preferably during REM sleep, more preferable during total REM sleep.

In certain embodiments, (relative) gamma power is determined at the dorsolateral prefrontal cortex and during total REM sleep.

In certain embodiments, (relative) gamma power is determined at the left and right cortex, preferably the left and right frontal cortex, more preferably the left and right prefrontal cortex, most preferably the left and right dorsolateral prefrontal cortex and during sleep, preferably during REM sleep, more preferable during total REM sleep.

In certain embodiments, (relative) gamma power is determined at the left and right dorsolateral prefrontal cortex and during total REM sleep.

It will be understood that for determining the relative gamma power, the percentage of gamma power over total brain wave power is determined over the same time period (e.g. during REM sleep) and at the same location (e.g. at the frontal cortex).

As used herein the term “Mini-Mental State Examination” or “MMSE” refers to the Folstein test as originally described in Folstein M F, Folstein S E, McHugh P R. “Mini-mental state”: a practical method for grading the cognitive state of patients for the clinician. J Psychiatr Res. (1975); 12(3):189-98. doi: 10.1016/0022-3956(75)90026-6; incorporated herein by reference in its entirety. It is used extensively in clinical and research settings to measure cognitive impairment, as is well known in the art. It is commonly used in medicine and allied health to screen for dementia. It is also used to estimate the severity and progression of cognitive impairment and to follow the course of cognitive changes in an individual over time; thus making it an effective way to document an individual's response to treatment.

The test is a 30-point questionnaire and administration of the test takes between 5 and 10 minutes and examines functions including registration (repeating named prompts), attention and calculation, recall, language, ability to follow simple commands and orientation. The MMSE test includes simple questions and problems in a number of areas: the time and place of the test, repeating lists of words, arithmetic such as the serial sevens, language use and comprehension, and basic motor skills. In certain preferred embodiments, the MMSE score as referred to herein is determined according to MMSE version 1 (i.e. the original edition), preferably standardized (Molloy and Standish (1997) “A guide to the standardized Mini-Mental State Examination” Int Psychogeriatr. 1997; 9 Suppl 1:87-94; discussion 143-50; incorporated herein by reference in its entirety).

Typically, the test is structured as follows:

| | Possible | |
|---|---|---|
| Category | points | Description |
| Orientation to | 5 | From broadest to most narrow. Orientation |
| time | | to time has been correlated with future |
| | | decline. |
| Orientation to | 5 | From broadest to most narrow. This is |
| place | | sometimes narrowed down to streets, and |
| | | sometimes to floor. |
| Registration | 3 | Repeating named prompts |
| Attention and | 5 | Serial sevens, or spelling “world” backwards. |
| calculation | | It has been suggested that serial sevens may |
| | | be more appropriate in a population where |
| | | English is not the first language. |
| Recall | 3 | Registration recall |
| Language | 2 | Naming a pencil and a watch |
| Repetition | 1 | Speaking back a phrase |
| Complex | 6 | Varies. Can involve drawing figure shown. |
| commands | | |

The results can be interpreted based on normative values for a person's age and education (see also O-Bryant et al. “Detecting dementia with the mini-mental state examination (MMSE) in highly educated individuals”. Arch Neurol. (2008); 65(7):963-967. doi:10.1001/archneur.65.7.963; incorporated herein by reference in its entirety. The following table provides median MMSE scores by age and educational level. The larger the difference between the patient's score and the age/education associated median, the more likely significant cognitive impairment exists. The skilled person will understand that depending on the age and/or education level of the subject, a different cut-off MMSE score may need to be applied. For instance, a subject having an MMSE score of 25 may be qualified to be treated according to the invention (and for instance being qualified as being at risk of developing a cognitive disorder) if having low education level and/or high age. However, a subject having an MMSE score of 25 may not be qualified to be treated according to the invention (and for instance being qualified as being at risk of developing a cognitive disorder) if having high education level and/or low age. In such case, an MMSE cut-off of 26 or 27 may be more appropriate for a subject to be qualified to be treated according to the invention.

| | Education | | | | |
|---|---|---|---|---|---|
| Age | 0-4 y | 5-8 y | 9-12 y | >=12 y | Total |
| 18-24 | 23 | 28 | 29 | 30 | 29 |
| 25-29 | 23 | 27 | 29 | 30 | 29 |
| 30-34 | 25 | 26 | 29 | 30 | 29 |
| 35-39 | 26 | 27 | 29 | 30 | 29 |
| 40-44 | 23 | 27 | 29 | 30 | 29 |
| 45-49 | 23 | 27 | 29 | 30 | 29 |
| 50-54 | 23 | 27 | 29 | 29 | 29 |
| 55-59 | 22 | 27 | 29 | 29 | 29 |
| 60-64 | 22 | 27 | 28 | 29 | 28 |
| 65-69 | 22 | 28 | 28 | 29 | 28 |
| 70-74 | 21 | 26 | 28 | 29 | 27 |
| 75-79 | 21 | 26 | 27 | 28 | 26 |
| 80-84 | 19 | 25 | 26 | 28 | 25 |
| >=85 | 20 | 24 | 26 | 28 | 25 |
| Total | 22 | 26 | 29 | 29 | 29 |

MMSE may be used to determine cognitive function or cognitive state. Cognitive functions are those mental processes that lead to the acquisition of knowledge and allow us to carry out our daily tasks. They allow the subject to have an active role in the processes of receiving, choosing, transforming, storing, processing and retrieval of information, allowing the subject to navigate the world around him. The most important cognitive functions are attention, orientation, memory, gnosis, executive functions, praxis, language, social cognition and visuospatial skills. Accordingly, in certain embodiments wherein the compositions or methods of the invention maintain or improve cognitive function, such may be determined by obtaining the MMSE score, wherein an improvement of cognitive function correlates with an improvement of MMSE score and wherein maintaining cognitive function correlates with maintaining an MMSE score or at least not a decrease of MMSE score. In certain embodiments, an improvement of cognitive function is an increase of the MMSE score with at least 1 point, preferably at least 2 points, such as at least 3 points. In certain embodiments, cognitive function is compared or evaluated after at least 4 weeks of treatment, such as at least 6 weeks of treatment, preferably at least 8 weeks of treatment, such as at least 12, 16, 20, 24, 28, or 32 weeks of treatment. In certain embodiments, the improvement of cognitive function (such as an increase in MMSE score) is higher in subjects (to be treated) according to the invention than in subjects not treated according to the invention. In certain embodiments, an improvement of cognitive function in subjects (to be treated) according to the invention is an increase of the MMSE score with at least 1 point more, preferably at least 2 points more, such as at least 3 points more than in subjects not treated according to the invention. In certain embodiments, the improvement of cognitive function (such as an increase in MMSE score) is faster in subjects (to be treated) according to the invention than in subjects not treated according to the invention. In certain embodiments, an improvement of cognitive function in subjects (to be treated) according to the invention is an increase of the MMSE score with at least 1 point, preferably at least 2 points, such as at least 3 points in a time frame which is at least 5%, preferably at least 10%, such as at least 20% shorter than in subjects not treated according to the invention. In certain embodiments, the improvement in cognitive function (such as an increase in MMSE score) is maintained over a longer time period in subjects (to be treated) according to the invention than in subjects not treated according to the invention. In certain embodiments, an improvement of cognitive function in subjects (to be treated) according to the invention is an increase of the MMSE score with at least 1 point, preferably at least 2 points, such as at least 3 points for a time period which is at least 5%, preferably at least 10%, such as at least 20% longer than in subjects not treated according to the invention.

As used herein, the term “qualified clinical restlessness event” preferably refers to a movement artefact during sleep (e.g. at night) or wake after sleep onset (WASO). The movement artefact may be associated with agitation. Underlying the movement artefact may be a sleep disorder, such as insomnia.

As used herein, the term “qualified clinical restlessness event during sleep” may be used interchangeously with “qualified clinical restlessness event during (a period of) wake after sleep onset” or “qualified clinical restlessness event during (a period of) sleep”.

In certain embodiments, the movement artefact is associated with and/or can be determined by occurrence of noise making of the subject, preferably at least 50 dB. Noise making can for instance be determined or detected by sound sensors.

In certain embodiments, the qualified clinical restlessness event does not include (sleep) apnea.

In certain embodiments, the movement artefact is associated with and/or can be determined by occurrence of (skeletal) muscle activity or activation, which can for instance be registered by an EMG and/or (infrared) video imaging. EMG measures the electrical activity of a muscle during rest, slight contraction, and forceful contraction.

Applied surface EMG assesses muscle function by recording muscle activity from the surface above the muscle on the skin and is recorded by a pair of electrodes. More than one electrode is applied because EMG recordings display the potential difference (voltage difference) between two separate electrodes. A healthy muscle typically will show no electrical activity (no signs of action potential) during rest, only when it contracts. As one contract a muscle more forcefully, more and more muscle fibers are activated, producing action potentials. So any signal on the EMG recording is detected as a movement artefact since during ‘normal’ rest there is typically no activity.

In certain embodiments, the movement artefact is associated with and/or can be determined by occurrence of noise making of the subject, preferably at least 50 dB; and muscle activity or activation, which can for instance be registered by an EMG and/or (infrared) video imaging.

As used herein, “during sleep” refers to the period starting from when a subject first falls asleep to when he becomes fully awake and does not attempt to go back to sleep. It corresponds to a period where a subject is supposed, expected, or scheduled to sleep or where the person desires to sleep. The skilled person will understand that such period may be and is preferably at night. However such period may equally be during daytime (e.g. in cases where a subject works during night-time). In any case, “during sleep” refers to a period following first falling asleep.

As used herein, the term “wake (time) after sleep onset” or “WASO” refers to the amount of time a person spends awake, starting from when they first fall asleep to when they become fully awake and do not attempt to go back to sleep. The normal unit of measure for this statistic is minutes. WASO can for instance be objectively determined by sleep studies such as polysomnography or EEG, in particular by the appearance of or increase in alpha waves, as is well known in the art.

The movement artefact can for instance be observed by an (infrared) video image, possibly associated with noise-making (preferably of at least 50 dB) and/or an EMG signal (which can also be part of polysomnography, as is well known in the art). As indicated above, the awake status can objectively detected by an EEG alpha signal which precedes, is part of or follows the period of movement artefact.

In certain embodiments, the movement artefact, preferably during wake after sleep onset (WASO), is determined by EEG and EMG.

In certain embodiments, the movement artefact, preferably during wake after sleep onset (WASO), is determined by EEG and (infrared) video imaging.

In certain embodiments, the movement artefact, preferably during wake after sleep onset (WASO), is determined by EEG and noise-making, preferably at least 50 dB.

In certain embodiments, the movement artefact, preferably during wake after sleep onset (WASO), is determined by EEG, EMG, and (infrared) video imaging.

In certain embodiments, the movement artefact, preferably during wake after sleep onset (WASO), is determined by EEG, EMG, and noise-making, preferably at least 50 dB.

In certain embodiments, the movement artefact, preferably during wake after sleep onset (WASO), is determined by EEG, (infrared) video imaging, and noise-making, preferably at least 50 dB.

In certain embodiments, the movement artefact, preferably during wake after sleep onset (WASO), is determined by EEG, EMG, (infrared) video imaging, and noise-making, preferably at least 50 dB.

In certain embodiments, the movement artefact is determined by EMG.

In certain embodiments, the movement artefact is determined by (infrared) video imaging.

In certain embodiments, the movement artefact is determined by noise-making, preferably at least 50 dB.

In certain embodiments, the movement artefact is determined by EMG and (infrared) video imaging.

In certain embodiments, the movement artefact is determined by EMG and noise-making, preferably at least 50 dB.

In certain embodiments, the movement artefact is determined by (infrared) video imaging and noise-making, preferably at least 50 dB.

In certain embodiments, the movement artefact is determined by EMG, (infrared) video imaging, and noise-making, preferably at least 50 dB.

In certain embodiments, the movement artefact (during wake after sleep onset) is determined by polysomnography.

In certain embodiments, the movement artefact (during wake after sleep onset) is determined by polysomnography and (infrared) video imaging.

Polysomnography (PSG), a type of sleep study, is a multi-parametric test used in the study of sleep and as a diagnostic tool in sleep medicine. The test result is called a polysomnogram, also abbreviated PSG. Polysomnography is a comprehensive recording of the biophysiological changes that occur during sleep. The PSG monitors many body functions, including brain activity (EEG), eye movements (EOG), muscle activity or skeletal muscle activation (EMG), and heart rhythm (ECG), during sleep.

By means of example, but without limitation, a polysomnogram will typically record a minimum of 12 channels requiring a minimum of 22 wire attachments to the patient. These channels vary in every lab and may be adapted to meet the doctors requests. There is a minimum of three channels for the EEG, one or two measure airflow, one or two are for chin muscle tone, one or more for leg movements, two for eye movements (EOG), one or two for heart rate and rhythm, one for oxygen saturation, and one each for the belts, which measure chest wall movement and upper abdominal wall movement. The movement of the belts is typically measured with piezoelectric sensors or respiratory inductance plethysmography. This movement is equated to effort and produces a low-frequency sinusoidal waveform as the patient inhales and exhales. Because movement is equated to effort, this system of measurement can produce false positives. It is possible, especially during obstructive apneas, for effort to be made without measurable movement.

Wires for each channel of recorded data lead from the patient and converge into a central box, which in turn is connected to a computer system for recording, storing and displaying the data. During sleep the computer monitor can display multiple channels continuously. In addition, most labs have a small video camera in the room so the technician can observe the patient visually from an adjacent room.

The electroencephalogram (EEG) will generally use six “exploring” electrodes and two “reference” electrodes, unless a seizure disorder is suspected, in which case more electrodes will be applied to document the appearance of seizure activity. The exploring electrodes are usually attached to the scalp near the frontal, central (top) and occipital (back) portions of the brain via a paste that will conduct electrical signals originating from the neurons of the cortex. These electrodes will provide a readout of the brain activity that can be “scored” into different stages of sleep (N1, N2, and N3—which combined are referred to as NREM sleep—and Stage R, which is rapid eye movement sleep, or REM, and Wakefulness). The EEG electrodes are placed according to the International 10-20 system.

The electrooculogram (EOG) uses two electrodes; one that is placed 1 cm above the outer canthus of the right eye and one that is placed 1 cm below the outer canthus of the left eye. These electrodes pick up the activity of the eyes in virtue of the electropotential difference between the cornea and the retina (the cornea is positively charged relative to the retina). This helps to determine when REM sleep occurs, of which rapid eye movements are characteristic, and also essentially aids in determining when sleep occurs.

The electromyogram (EMG) typically uses four electrodes to measure muscle tension in the body as well as to monitor for an excessive amount of leg movements during sleep (which may be indicative of periodic limb movement disorder, PLMD). Two leads are placed on the chin with one above the jawline and one below. This, like the EOG, helps determine when sleep occurs as well as REM sleep. Sleep generally includes relaxation and so a marked decrease in muscle tension occurs. A further decrease in skeletal muscle tension occurs in REM sleep. A person becomes partially paralyzed to make acting out of dreams impossible, although people that do not have this paralysis can suffer from REM behaviour disorder. Finally, two more leads are placed on the anterior tibialis of each leg to measure leg movements.

Though a typical electrocardiogram (ECG or EKG) would use ten electrodes, only two or three are used for a polysomnogram. They can either be placed under the collarbone on each side of the chest or one under the collarbone and the other six inches above the waist on either side of the body. These electrodes measure the electrical activity of the heart as it contracts and expands, recording such features as the “P” wave, “QRS” complex, and “T” wave. These can be analyzed for any abnormalities that might be indicative of an underlying heart pathology.

Nasal and oral airflow can be measured using pressure transducers, and/or a thermocouple, fitted in or near the nostrils; the pressure transducer is considered the more sensitive. This allows the clinician/researcher to measure the rate of respiration and identify interruptions in breathing. Respiratory effort is also measured in concert with nasal/oral airflow by the use of belts. These belts expand and contract upon breathing effort. However, this method of respiration may also produce false positives. Some patients will open and close their mouth while obstructive apneas occur. This forces air in and out of the mouth while no air enters the airway and lungs. Thus, the pressure transducer and thermocouple will detect this diminished airflow and the respiratory event may be falsely identified as a hypopnea, or a period of reduced airflow, instead of an obstructive apnea.

Pulse oximetry determines changes in blood oxygen levels that often occur with sleep apnea and other respiratory problems. The pulse oximeter fits over a fingertip or an earlobe.

Snoring may be recorded with a sound probe over the neck, though more commonly the sleep technician will just note snoring as “mild”, “moderate” or “loud” or give a numerical estimate on a scale of 1 to 10. Also, snoring indicates airflow and can be used during hypopneas to determine whether the hypopnea may be an obstructive apnea.

In will be understood that not all parameters of PSG are relevant in the context of the present invention. The most relevant parameters are the EEG and EMG.

As used herein, the term “subject” preferably refers to a subject, preferably human, in need of the prophylactic or therapeutic treatments of the invention as described herein or which will benefit from the prophylactic or therapeutic treatments of the invention as described herein.

In certain embodiments, the subjects to be prophylactically or therapeutically treated according to the invention have an increased risk of developing a cognitive disorder or an increased risk of cognitive disorder progression or worsening.

Risk factors for developing a cognitive disorder include (medical history of) major depression, diabetes mellitus, renal function impairment, hypertension, hypercholesterolemia, hyperlipidemia, cardio-vascular disease (including coronary artery disease, atrial fibrillations, heart failure, or valvular heart disease), cerebro-vascular disease, gastrointestinal disorder, anemia, inflammation, oxidative stress, chronic obstructive pulmonary disease, liver function impairment (including cirrhosis), sleep disorder or any medical condition which is related to an increased incidence of developing a cognitive disorder, but also genetic predisposition, age, sex, education, etc.

Accordingly, in certain embodiments, the subject has one or more of (medical history of) major depression, diabetes mellitus, renal function impairment, hypertension, hypercholesterolemia, hyperlipidemia, cardio-vascular disease (including coronary artery disease, atrial fibrillations, heart failure, or valvular heart disease), cerebro-vascular disease, gastrointestinal disorder, anemia, inflammation, oxidative stress, chronic obstructive pulmonary disease, liver function impairment (including cirrhosis), sleep disorder or any medical condition which is related to an increased incidence of developing a cognitive disorder, but also genetic predisposition, age above 60, preferably above 65.

In certain embodiments, the subjects to be treated according to the invention have a (medical history) of affective disorder, preferably (mild) major depressive disorder, preferably during adult life, preferably as specified in and according to the criteria of DSM-5.

It will be understood that “medical history of affective disorder” refers to the occurrence of such affective disorder (such as major depressive disorder) during the lifetime of the subject, preferably during adult life of the subject (such as from 16, 17, or 18 years of age onward). Included are recurrent or remittent affective disorders. In certain embodiments, a subject having a medical history of affective disorder has an affective disorder, i.e. at the time of initiating the treatment according to the invention as described herein the subject is afflicted with an affective disorder. In certain embodiments, a subject having a medical history of affective disorder does not have an affective disorder, i.e. at the time of initiating the treatment according to the invention as described herein the subject is not afflicted with an affective disorder or is in (full or partial) remission. In certain embodiments, a subject having an affective disorder does not have a medical history of an affective disorder, i.e. at the time of initiating the treatment according to the invention as described herein the subject does not have a medical history of an affective disorder, i.e. the affective disorder is not recurrent (at the time of diagnosis). In certain embodiments, a subject having an affective disorder has a medical history of an affective disorder, i.e. at the time of initiating the treatment according to the invention as described herein the subject also has a medical history of an affective disorder. In certain embodiments, the subjects to be treated according to the invention have an affective disorder in remission. In certain embodiments, the subjects to be treated according to the invention have major depressive disorder in remission. It will be understood that the subjects to be treated according to the invention, when having (a medical history op an affective disorder (such as major depressive disorder), may simultaneously receive therapy for an affective disorder (such as major depressive disorder). In certain embodiments, the subjects to be treated according to the invention receive a combination of an effective amount of an antidepressant and a D4 (and 5-HT2A) receptor antagonist, reverse agonist, or partial agonist, such as pipamperone, preferably in a daily dose of 5-20 mg or 4 to 20 mg.

By means of further guidance, and without limitation, affective disorders (or mood disorders or affective spectrum (disorder)) include attention deficit hyperactivity disorder, bipolar disorder, body dysmorphic disorder, bulimia nervosa and other eating disorders, cataplexy, dysthymia, generalized anxiety disorder, hypersexuality, irritable bowel syndrome, impulse-control disorders, kleptomania, migraine, major depressive disorder, obsessive-compulsive disorder, oppositional defiant disorder, panic disorder, posttraumatic stress disorder, premenstrual dysphoric disorder, social anxiety disorder, fibromyalgia, chronic pain, intermittent explosive disorder, pathological gambling, personality disorder, pyromania, substance abuse and addiction (includes alcoholism), trichotillomania.

In certain embodiments, the affective disorder is selected from attention deficit hyperactivity disorder, bipolar disorder, body dysmorphic disorder, bulimia nervosa and other eating disorders, cataplexy, dysthymia, generalized anxiety disorder, hypersexuality, irritable bowel syndrome, impulse-control disorders, kleptomania, migraine, major depressive disorder, obsessive-compulsive disorder, oppositional defiant disorder, panic disorder, posttraumatic stress disorder, premenstrual dysphoric disorder, social anxiety disorder, fibromyalgia, chronic pain, intermittent explosive disorder, pathological gambling, personality disorder, pyromania, substance abuse and addiction (includes alcoholism), trichotillomania.

In certain embodiments, the affective disorder is selected from attention deficit hyperactivity disorder, bipolar disorder, body dysmorphic disorder, bulimia nervosa and other eating disorders, cataplexy, dysthymia, generalized anxiety disorder, hypersexuality, irritable bowel syndrome, impulse-control disorders, kleptomania, migraine, major depressive disorder, obsessive-compulsive disorder, oppositional defiant disorder, panic disorder, posttraumatic stress disorder, premenstrual dysphoric disorder, social anxiety disorder, fibromyalgia.

In certain embodiments, the affective disorder is selected from (major) depressive disorder, bipolar disorder, and anxiety disorder. In a preferred embodiment, the affective disorder is major depressive disorder.

Major depressive disorder (MDD) can be diagnosed according to the criteria for MDD in DSM-5 (Diagnostic and Statistical Manual of Mental Disorders). MDD in full remission is defined as no significant MDD symptoms being present directly following the most recent depressed episode for at least 2 months directly following the most recent depressed episode. This can be evaluated based upon clinical assessment and confirmed by the Mini International Neuropsychiatric Interview (MINI) and the attending psychiatrist. Partial remission can be the persistence of residual symptoms (without meeting the criteria for a diagnosis of MDD) or can be the absence of significant MDD symptoms for a period of less than two months following the most recent depressed episode. Remission can also be determined by an IDS-C30 total score of less than 34 (30 item Inventory of Depressive Symptomatology; Rush et al. (1986), Psychiatry Research, 18:65-87; Rush et al. (1996), Psychological Medicine, 26:477-486). Preferably, MDD is evaluated according to the Montgomery-Asberg Depression Rating Scale (MADRS). The Montgomery-Asberg Depression Rating Scale (MADRS) is a 10-item clinician-administered measure indication the severity of the depressive symptoms within a subject and with a maximum total score of 60. The MADRS contains following items and related scores:

1. Apparent Sadness

Representing despondency, gloom and despair, (more than just ordinary transient low spirits) reflected in speech, facial expression, and posture. Rate by depth and inability to brighten up.
(0) No sadness.
(1)
(2) Looks dispirited but does brighten up without difficulty.
(3)
(4) Appears sad and unhappy most of the time.
(5)
(6) Looks miserable all the time. Extremely despondent.

2. Reported Sadness

Representing reports of depressed mood, regardless of whether it is reflected in appearance or not. Includes low spirits, despondency or the feeling of being beyond help and without hope. Rate according to intensity, duration and the extent to which the mood is reported to be influenced by events.
(0) Occasional sadness in keeping with the circumstances.
(1)
(2) Sad or low but brightens up without difficulty.
(3)
(4) Pervasive feelings of sadness or gloominess. The mood is still influenced by external circumstances.
(5)
(6) Continuous or unvarying sadness, misery or despondency.

3. Inner Tension

Representing feelings of ill-defined discomfort, edginess, inner turmoil, mental tension mounting to either panic, dread or anguish. Rate according to intensity, frequency, duration and the extent of reassurance called for.
(0) Placid. Only fleeting inner tension.
(1)
(2) Occasional feelings of edginess and ill-defined discomfort.
(3)
(4) Continuous feelings of inner tension or intermittent panic which the patient can only master with some difficulty.
(5)
(6) Unrelenting dread or anguish. Overwhelming panic.

4. Reduced Sleep

Representing the experience of reduced duration or depth of sleep compared to the subject's own normal pattern when well.
(0) Sleeps as usual.
(1)
(2) Slight difficulty dropping off to sleep or slightly reduced, light or fitful sleep.
(3)
(4) Sleep reduced or broken by at least two hours.
(5)
(6) Less than two or three hours sleep.

5. Reduced Appetite

Representing the feeling of a loss of appetite compared with when well. Rate by loss of desire for food or the need to force oneself to eat.
(0) Normal or increased appetite.
(1)
(2) Slightly reduced appetite.
(3)
(4) No appetite. Food is tasteless.
(5)
(6) Needs persuasion to eat at all.

6. Concentration Difficulties Representing difficulties in collecting one's thoughts mounting to incapacitating lack of concentration. Rate according to intensity, frequency, and degree of incapacity produced.
(0) No difficulties in concentrating.
(1)
(2) Occasional difficulties in collecting one's thoughts.
(3)
(4) Difficulties in concentrating and sustaining thought which reduces ability to read or hold a conversation.
(5)
(6) Unable to read or converse without great difficulty.

7. Lassitude

Representing a difficulty getting started or slowness initiating and performing everyday activities.
(0) Hardly any difficulties in getting started. No sluggishness.
(1)
(2) Difficulties in starting activities.
(3)
(4) Difficulties in starting simple routine activities, which are carried out with effort.
(5)
(6) Complete lassitude. Unable to do anything without help.

8. Inability to Feel

Representing the subjective experience of reduced interest in the surroundings, or activities that normally give pleasure. The ability to react with adequate emotion to circumstances or people is reduced.
(0) Normal interest in the surroundings and in other people.
(1)
(2) Reduced ability to enjoy usual interests.
(3)
(4) Loss of interest in the surroundings. Loss of feelings for friends and acquaintances.
(5)
(6) The experience of being emotionally paralyzed, inability to feel anger, grief or pleasure and a complete or even painful failure to feel for close relatives and friends.

9. Pessimistic Thoughts

Representing thoughts of guilt, inferiority, self-reproach, sinfulness, remorse and ruin.
(0) No pessimistic thoughts.
(1)
(2) Fluctuating ideas of failure, self-reproach or self-depreciation.
(3)
(4) Persistent self-accusations, or definite but still rational ideas of guilt or sin. Increasingly pessimistic about the future.
(5)
(6) Delusions of ruin, remorse and irredeemable sin. Self-accusations which are absurd and unshakable.

10. Suicidal Thoughts

Representing the feeling that life is not worth living, that a natural death would be welcome, suicidal thoughts, and preparations for suicide. Suicidal attempts should not in themselves influence the rating.
(0) Enjoys life or takes it as it comes.
(1)
(2) Weary of life. Only fleeting suicidal thoughts.
(3)
(4) Probably better off dead. Suicidal thoughts are common, and suicide is considered as a possible solution, but without specific plans or intention.
(5)
(6) Explicit plans for suicide when there is an opportunity. Active preparations for suicide.

A subject is considered to be in remission (or a subject is considered not to have MDD (anymore)) if the total MADRS score is ≤10. A subject is considered to have mild depressive symptoms (or has mild (major) depressive disorder) as indicated by a total score of 18 or less on the Montgomery-Asberg Depression Rating Scale (MADRS). Accordingly, in certain embodiments, a subject having mild depressive symptoms or a subject having mild MDD or having MDD with mild (depressive) symptoms has a total score ranging from 11 to 18 on the Montgomery-Asberg Depression Rating Scale (MADRS). In certain embodiments, a subject not having MDD (anymore) or having MDD in remission has a total score ≤10 on the Montgomery-Asberg Depression Rating Scale (MADRS). In certain embodiments, a subject having MDD has a total score >10 on the Montgomery-Asberg Depression Rating Scale (MADRS).

Suicidal thoughts (or suicidality) can be evaluated according to item 10 of the MADRS. A reduction of suicidal thoughts can be evaluated as a reduction in the score of item 10 of the MADRS.

Cognitive function can be evaluated according to item 6 of the MADRS. An improvement or enhancement of cognitive function can be evaluated as a reduction in the score of item 6 of the MADRS.

Medication for treating affective disorders, as well as suitable effective dosage regimes are well known in the art. By means of example, and without limitation, medication of treating affective disorders, in particular major depressive disorder, include selective serotonin reuptake inhibitors (SSRI) (such as citalopram (e.g. Celexa), escitalopram (e.g. Lexapro), fluoxetine (e.g. Prozac), fluvoxamine (e.g. Luvox), paroxetine (e.g. Paxil), sertraline (e.g. Zoloft), dapoxetine (e.g. Prilligy), indalpine (e.g. Upstène), zimelidine (e.g. Zelmid), alaproclate (GEA-654), centpropazine, cericlamine (JO-1017), femoxetine (Malexil; FG-4963), ifoxetine (CGP-15210), omiloxetine, panuramine (WY-26002), pirandamine (AY-23713), seproxetine ((S)-norfluoxetine)), serotonin-norepinephrine reuptake inhibitors (SNRI) (such as atomoxetine (e.g. Strattera), desvenlafaxine (e.g. Pristiq, Khedezla), duloxetine (e.g. Cymbalta, Irenka), levomilnacipran (e.g. Fetzima), milnacipran (e.g. Ixel, Savella, Impulsor), sibutramine (e.g. Meridia), tramadol (e.g. Ultram), venlafaxine (e.g. Effexor)), serotonin modulators and stimulators (SMS) (such as vortioxetine, vilazodone), serotonin antagonists and reuptake inhibitors (SARI) (such as etoperidone (e.g. Axiomin, Etonin), lorpiprazole (e.g. Normarex), mepiprazole (e.g. Psigodal), nefazodone (e.g. Serzone, Nefadar), trazodone (e.g. Desyrel), vilazodone (e.g. Viibryd), vortioxetine (e.g. Trintellix), niaprazine (e.g. Nopron), medifoxamine (e.g.Cledial, Gerdaxyl), lubazodone), norepinephrine reuptake inhibitors (NRI or NERI) (such as amedalin (UK-3540-1), atomoxetine (e.g. Strattera), CP-39,332, daledalin (UK-3557-15), edivoxetine (LY-2216684), esreboxetine, lortalamine (LM-1404), nisoxetine (LY-94,939), reboxetine (e.g. Edronax, Vestra), talopram (e.g. tasulopram) (Lu 3-010), talsupram (Lu 5-005), tandamine (AY-23,946), viloxazine (Vivalan), including NRIs with activity at other sites such as bupropion (e.g. Wellbutrin, Zyban), ciclazindol (Wy-23,409), duloxetine, manifaxine (GW-320,659), maprotiline (e.g. Deprilept, Ludiomil, Psymion), radafaxine (GW-353,162), tapentadol (e.g. Nucynta), teniloxazine (e.g. Lucelan, Metatone), protriptyline (e.g. Vivactil), nortriptyline (e.g. Pamelor), desipramine (e.g. Norpramin)), norepinephrine-dopamine reuptake inhibitors (NDRI) (such as bupropion), tricyclic antidepressants (TCA) (such as butriptyline (e.g. Evadyne), clomipramine (e.g. Anafranil), imipramine (e.g. Tofranil, Janimine, Praminil), trimipramine (e.g. Surmontil), desipramine (e.g. Norpramin, Pertofrane), dibenzepin (e.g. Noveril, Victoril), lofepramine (e.g. Lomont, Gamanil), maprotiline (e.g. Ludiomil), nortriptyline (e.g. Pamelor, Aventyl, Norpress), protriptyline (e.g.Vivactil), amitriptyline (e.g.Elavil, Endep), amitriptylinoxide (e.g.Amioxid, Ambivalon, Equilibrin), amoxapine (e.g. Asendin), demexiptiline (e.g. Deparon, Tinoran), dimetacrine (e.g. Istonil, Istonyl, Miroistonil), dosulepin (e.g. Prothiaden), doxepin (e.g. Adapin, Sinequan), fluacizine (e.g. Phtorazisin), imipraminoxide (e.g. Imiprex, Elepsin), melitracen (e.g. Deanxit, Dixeran, Melixeran, Trausabun), metapramine (e.g. Timaxel), nitroxazepine (e.g. Sintamil), noxiptiline (e.g. Agedal, Elronon, Nogedal), pipofezine (e.g. Azafen/Azaphen), propizepine (e.g. Depressin, Vagran), quinupramine (e.g. Kevopril, Kinupril, Adeprim, Quinuprine), amineptine (e.g. Survector, Maneon, Directim), iprindole (e.g. Prondol, Galatur, Tetran), opipramol (e.g. Insidon, Pramolan, Ensidon, Oprimol), tianeptine), tetracyclic antidepressants (TeCA) (such as maprotiline (e.g. Ludiomil), mianserin (e.g. Tolvon), mirtazapine (e.g. Remeron), setiptiline (e.g. Tecipul), amoxapine (e.g. Asendin), benzoctamine (e.g. Tacitin), loxapine (e.g. Adasuve, Loxitane), mazindol (e.g. Mazanor, Sanorex, aptazapine (CGS-7525A), esmirtazapine (ORG-50,081), oxaprotiline (C 49-802 BDA), ciclazindol (WY-23,409)), monoamine oxidase inhibitors (MAOI) (such as isocarboxazid (e.g. Marplan), nialamide (e.g. Niamid), phenelzine (e.g. Nardil, Nardelzine), hydracarbazine, tranylcypromine (e.g. Parnate, Jatrosom), bifemelane (e.g. Alnert, Celeport), moclobemide (e.g. Aurorix, Manerix), pirlindole (e.g. Pirazidol, toloxatone (e.g. Humoryl), rasagiline (e.g. Azilect), selegiline (e.g. Deprenyl, Eldepryl, Emsam, Zelapar), safinamide (e.g. Xadago), linezolid, benmoxin (e.g. Nerusil, Neuralex), iproclozide (e.g. Sursum), iproniazid (e.g. Marsilid, Iprozid, Ipronid, Rivivol, Propilniazida), mebanazine (e.g. Actomol), octamoxin (e.g. Ximaol, Nimaol), pheniprazine (e.g. Catron), phenoxypropazine (e.g. Drazine), pivalylbenzhydrazine (e.g. Tersavid), safrazine (e.g. Safra), caroxazone (e.g. Surodil, Timostenil), minaprine (e.g. Cantor), brofaromine (e.g. Consonar), caroxazone (e.g. Surodil, Timostenil), eprobemide (e.g. Befol), methylene blue, metralindole (e.g. Inkazan), minaprine (e.g. Cantor), moclobemide (e.g. Aurorix, Manerix), pirlindole (e.g. Pirazidol), toloxatone (e.g. Humoryl), curcumin, harmaline, harmine, amiflamine (FLA-336), befloxatone (MD-370,503), cimoxatone (MD-780,515), esuprone, sercloremine (CGP-4718-A), tetrindole, CX157 (TriRima)), and NMDA receptor antagonists (e.g. ketamine, esketamine).

Psychosis is characterized by a loss of contact with reality. The symptoms of psychosis include delusions, or false beliefs and false perceptions, and hallucinations, or seeing and hearing things that aren't present, and can be assessed by the MINI (Mini International Neuropsychiatric Interview).

In certain embodiments, the invention relates to a D4 (and preferably also 5-HT2A) receptor antagonist, reverse agonist, or partial agonist as described herein elsewhere in combination with an antidepressant as described herein elsewhere for use in preventing, delaying or postponing the onset or delaying or postponing progression, and/or treating of a cognitive disorder or an affective disorder, preferably major depressive disorder, and/or for preventing suicide or preventing or reducing suicidal thoughts in a subject having (a medical history of) an affective disorder, preferably (mild) major depressive disorder, and/or for use in maintaining or improving cognitive function and/or for preventing suicide or preventing or reducing suicidal thoughts in a subject having (a medical history of) an affective disorder, preferably (mild) major depressive disorder, and/or for use in maintaining or increasing gamma power, preferably relative gamma power, preferably at the cortex, preferably at the frontal cortex, such as the (dorsolateral) pre-frontal cortex, preferably during sleep, preferably REM sleep.

In certain embodiments, the invention relates to a D4 (and preferably also 5-HT2A) receptor antagonist, reverse agonist, or partial agonist as described herein elsewhere for use in preventing, delaying or postponing the onset or delaying or postponing progression, and/or treating of a cognitive disorder, or an affective disorder, preferably (mild) major depressive disorder, and/or for preventing suicide or preventing or reducing suicidal thoughts in a subject having (a medical history of) an affective disorder, preferably (mild) major depressive disorder which is treated with an antidepressant as described herein elsewhere, and/or for use in maintaining or improving cognitive function, and/or for preventing suicide or preventing or reducing suicidal thoughts in a subject having (a medical history of) an affective disorder, preferably (mild) major depressive disorder which is treated with an antidepressant as described herein elsewhere, and/or for use in maintaining or increasing gamma power, preferably relative gamma power, preferably at the cortex, preferably at the frontal cortex, such as the (dorsolateral) pre-frontal cortex, preferably during sleep, preferably REM sleep.

It will be understood that the combination of the D4 (and preferably also 5-HT2A) receptor antagonist, reverse agonist, or partial agonist and the antidepressant may be a single composition or may be separate compositions and/or may be for simultaneous or subsequent administration. The skilled person will understand that the term “combination” in this context refers to at least partially overlapping treatment regimens over time or that the subject to be treated for a specified period is to be treated during at least part of that period both with the D4 (and preferably also 5-HT2A) receptor antagonist, reverse agonist, or partial agonist and the antidepressant. The skilled person will understand that the D4 (and preferably also 5-HT2A) receptor antagonist, reverse agonist, or partial agonist and the antidepressant may or may not be administered at the same time. The skilled person will understand that the D4 (and preferably also 5-HT2A) receptor antagonist, reverse agonist, or partial agonist and the antidepressant may or may not be administered at the same frequency (e.g. one drug may be administered daily while the other drug may also be administered daily or may for instance be administered bidaily or during the first weeks biweekly and after a defined period once every two weeks).

In certain embodiments, the subjects at risk of developing a cognitive disorder (to be treated according to the invention) have subjective cognitive decline, (pre-)mild cognitive impairment, a (pre-)prodromal neurodegenerative disease, preferably (pre-)prodromal Alzheimer's disease, and/or a pre-clinical or asymptomatic neurodegenerative disease, preferably pre-clinical or asymptomatic Alzheimer's disease, as defined herein elsewhere in this specification. In certain embodiments, the subjects at risk of developing a cognitive disorder (to be treated according to the invention) has not been diagnosed with Alzheimer's disease. In certain embodiments, the subjects at risk of developing a cognitive disorder (to be treated according to the invention) has been diagnosed with Alzheimer's disease. In certain embodiments, the subjects at risk of developing a cognitive disorder (to be treated according to the invention) has positive AD biomarkers, for instance for amyloidosis (neuropathologic β-amyloid plaque and tau accumulation) and neurodegeneration. In certain embodiments, the subjects at risk of developing a cognitive disorder (to be treated according to the invention) has negative AD biomarkers or does not have positive AD biomarkers.

In certain embodiments, the subjects to be treated according to the invention are at least 18 years old, preferably at least 60 years old, more preferably at least 65 years old, most preferably at least 70 years old.

In certain embodiments, the cognitive disorder is associated with or characterized by an MMSE score of less than 27, preferably less than 26, more preferably less than 25. In certain embodiments, a subject having a cognitive disorder has an MMSE score of less than 27, preferably less than 26, more preferably less than 25.

In certain embodiments, the subjects to be treated according to the invention have an MMSE score ranging from 27 to 30, preferably ranging from 27 to 29, such as 28 to 29.

In certain embodiments, the subjects to be treated according to the invention have an MMSE score ranging from 26 to 30, preferably ranging from 26 to 29.

In certain embodiments, the subjects to be treated according to the invention have an MMSE score ranging from 25 to 30, preferably ranging from 25 to 29.

In certain embodiments, an MMSE score ranging from 25 to 29, 26 to 29, or 27-29 is considered a risk factor for developing a cognitive disorder.

In certain embodiments, the subjects to be treated according to the invention are characterised as having at least 30, preferably at least 40, more preferably at least 50 minutes wake after sleep onset (WASO).

In certain embodiments, the subjects to be treated according to the invention have at least 1, preferably at least 2, more preferably at least 3 clinical relevant restlessness event (during sleep or at night) and/or sleep disorder such as insomnia.

In certain embodiments, the subjects to be treated according to the invention have an MMSE score ranging from 25, 26, or 27 to 30, preferably ranging from 25, 26, or 27 to 29; and have at least 1, preferably at least 2, more preferably at least 3 clinical relevant restlessness event (during sleep or at night) and/or sleep disorder such as insomnia.

In certain embodiments, the subjects to be treated according to the invention have an MMSE score ranging from 25, 26, or 27 to 30, preferably ranging from 25, 26, or 27 to 29; and have at least 30, preferably at least 40, more preferably at least 50 minutes wake after sleep onset (WASO).

In certain embodiments, the subjects to be treated according to the invention have an MMSE score ranging from 25, 26, or 27 to 30, preferably ranging from 25, 26, or 27 to 29; and have a (medical history of) affective disorder, preferably (mild) major depressive disorder.

In certain embodiments, the subjects to be treated according to the invention have at least 1, preferably at least 2, more preferably at least 3 clinical relevant restlessness event (during sleep or at night) and/or sleep disorder such as insomnia; and have at least 30, preferably at least 40, more preferably at least 50 minutes wake after sleep onset (WASO).

In certain embodiments, the subjects to be treated according to the invention have at least 1, preferably at least 2, more preferably at least 3 clinical relevant restlessness event (during sleep or at night) and/or sleep disorder such as insomnia; and have a (medical history of) affective disorder, preferably (mild) major depressive disorder.

In certain embodiments, the subjects to be treated according to the invention are characterised as having at least 30, preferably at least 40, more preferably at least 50 minutes wake after sleep onset (WASO); and have a (medical history of) affective disorder, preferably (mild) major depressive disorder.

In certain embodiments, the subjects to be treated according to the invention have an MMSE score ranging from 25, 26, or 27 to 30, preferably ranging from 25, 26, or 27 to 29; and have at least 1, preferably at least 2, more preferably at least 3 clinical relevant restlessness event (during sleep or at night) and/or sleep disorder such as insomnia; and have at least 30, preferably at least 40, more preferably at least 50 minutes wake after sleep onset (WASO).

In certain embodiments, the subjects to be treated according to the invention have an MMSE score ranging from 25, 26, or 27 to 30, preferably ranging from 25, 26, or 27 to 29; and have at least 1, preferably at least 2, more preferably at least 3 clinical relevant restlessness event (during sleep or at night) and/or sleep disorder such as insomnia; and have a (medical history of) affective disorder, preferably (mild) major depressive disorder.

In certain embodiments, the subjects to be treated according to the invention have an MMSE score ranging from 25, 26, or 27 to 30, preferably ranging from 25, 26, or 27 to 29; and have at least 30, preferably at least 40, more preferably at least 50 minutes wake after sleep onset (WASO); and have a (medical history of) affective disorder, preferably (mild) major depressive disorder.

In certain embodiments, the subjects to be treated according to the invention have at least 30, preferably at least 40, more preferably at least 50 minutes wake after sleep onset (WASO); and have a (medical history of) affective disorder, preferably (mild) major depressive disorder; and have at least 1, preferably at least 2, more preferably at least 3 clinical relevant restlessness event (during sleep or at night) and/or sleep disorder such as insomnia.

In certain embodiments, the subjects to be treated according to the invention have an MMSE score ranging from 25, 26, or 27 to 30, preferably ranging from 25, 26, or 27 to 29; and have at least 1, preferably at least 2, more preferably at least 3 clinical relevant restlessness event (during sleep or at night) and/or sleep disorder such as insomnia; and have (a medical history of) affective disorder, preferably (mild) major depressive disorder; and have at least 30, preferably at least 40, more preferably at least 50 minutes wake after sleep onset (WASO).

As used herein, the term “sleep disorder” has its ordinary meaning known in the art. By means of further guidance, and without limitation, a sleep disorder can be selected from, may be caused by, or may be associated with:

Dyssomnias—A broad category of sleep disorders characterized by either hypersomnia or insomnia. The three major subcategories include intrinsic (i.e., arising from within the body), extrinsic (secondary to environmental conditions or various pathologic conditions), and disturbances of circadian rhythm.
Insomnia: Insomnia may be primary or it may be comorbid with or secondary to another disorder such as a mood disorder (i.e., emotional stress, anxiety, depression) or underlying health condition (i.e., asthma, diabetes, heart disease, pregnancy or neurological conditions).
Primary hypersomnia. Hypersomnia of central or brain origin.
   Narcolepsy: A chronic neurological disorder (or dyssomnia), which is caused by the brain's inability to control sleep and wakefulness.
   Idiopathic hypersomnia: a chronic neurological disease similar to narcolepsy in which there is an increased amount of fatigue and sleep during the day. Patients who suffer from idiopathic hypersomnia cannot obtain a healthy amount of sleep for a regular day of activities. This hinders the patients' ability to perform well, and patients have to deal with this for the rest of their lives.
   Recurrent hypersomnia—including Kleine—Levin syndrome
   Posttraumatic hypersomnia
   Menstrual-related hypersomnia
Sleep disordered breathing (SDB), including (non exhaustive):
   Several types of Sleep apnea
   Snoring
   Upper airway resistance syndrome
Restless leg syndrome
Periodic limb movement disorder

Circadian rhythm sleep disorders
Delayed sleep phase disorder
Advanced sleep phase disorder
Non-24-hour sleep—wake disorder

Parasomnias—A category of sleep disorders that involve abnormal and unnatural movements, behaviors, emotions, perceptions, and dreams in connection with sleep.
Bedwetting or sleep enuresis
Bruxism (Tooth-grinding)
Catathrenia—nocturnal groaning
Exploding head syndrome—Waking up in the night hearing loud noises.
Sleep terror (or Pavor nocturnus)—Characterized by a sudden arousal from deep sleep with a scream or cry, accompanied by some behavioral manifestations of intense fear.
REM sleep behaviour disorder
Sleepwalking (or somnambulism)
Sleep talking (or somniloquy)
Sleep sex (or sexsomnia)

Medical or psychiatric conditions that may produce sleep disorders
22q11.2 deletion syndrome
Alcoholism
Mood disorders
   Depression
Anxiety disorder
Panic
Psychosis (such as Schizophrenia)

Preferably, the sleep disorder as used herein is as defined according to DSM-5 (Diagnostic and Statistical Manual of Mental Disorders).

In certain embodiments, the subject at risk of developing a cognitive disorder has a sleep disorder. Preferably, the sleep disorder is insomnia. In certain embodiments, the insomnia is acute insomnia. In certain embodiments, the insomnia is chronic insomnia. In certain embodiments, the insomnia is primary insomnia. In certain embodiments, the insomnia is secondary insomnia.

Insomnia is a sleep disorder that is characterized by difficulty falling and/or staying asleep. People with insomnia have one or more of the following symptoms: difficulty falling asleep, waking up often during the night and having trouble going back to sleep, waking up too early in the morning, and/or feeling tired upon waking. There are two types of insomnia: primary insomnia and secondary insomnia. Primary insomnia means that a person is having sleep problems that are not directly associated with any other health condition or problem. Secondary insomnia means that a person is having sleep problems because of something else, such as a health condition (like asthma, depression, arthritis, cancer, or heartburn); pain; medication they are taking; or a substance they are using (like alcohol). Insomnia also varies in how long it lasts and how often it occurs. It can be short-term (acute insomnia) or can last a long time (chronic insomnia). It can also come and go, with periods of time when a person has no sleep problems. Acute insomnia can last from one night to a few weeks. Insomnia is called chronic when a person has insomnia at least three nights a week for a month or longer.

The present invention also encompasses pharmaceutical compositions comprising the D4 (and 5-HT2A) receptor antagonist, reverse agonist, or partial agonist, preferably in admixture with one or more suitable pharmaceutically acceptable excipient.

The present invention also encompasses the use of such pharmaceutical compositions in the methods as described herein.

The present invention also encompasses such pharmaceutical compositions for use in the methods as described herein.

The present invention also encompasses the use of such pharmaceutical compositions for the manufacture of a medicament for use in the methods as described herein.

To prepare the pharmaceutical compositions, an effective amount of the active ingredients, for instance in acid or base addition salt form or base form, is combined in admixture with a pharmaceutically acceptable carrier, which can take a wide variety of forms depending on the form of preparation desired for administration. These pharmaceutical compositions are desirably in unitary dosage form suitable, for administration orally, nasal, rectally, percutaneously, transdermally, by parenteral, intramuscular, intravascular injection or intrathecal administration. For example, in preparing the compositions in oral dosage form, any of the usual pharmaceutical media may be employed, such as, for example, water, glycols, oils, alcohols and the like in the case of oral liquid preparations such as suspensions, syrups, elixirs and solutions; or solid carriers such as starches, sugars, kaolin, lubricants, binders, disintegrating agents and the like in the case of powders, pills, capsules and tablets. Because of their ease in administration, tablets and capsules represent the most advantageous oral dosage unit form, in which case solid pharmaceutical carriers are obviously employed. For parenteral compositions, the carrier will usually comprise sterile water, at least in large part, though other ingredients, for example, to aid solubility, may be included.

The pharmaceutical compounds for treatment are intended for parenteral, topical, oral or local administration and generally comprise a pharmaceutically acceptable carrier and an amount of the active ingredient sufficient to reverse or prevent the bad effects of mental disorders. The carrier may be any of those conventionally used and is limited only by chemico-physical considerations, such as solubility and lack of reactivity with the compound, and by the route of administration.

Examples of pharmaceutically acceptable acid addition salts for use in the present inventive pharmaceutical composition include those derived from mineral acids, such as hydrochloric, hydrobromic, phosphoric, metaphosphoric, nitric and sulfuric acids, and organic acids, such as tartaric, acetic, citric, malic, lactic, fumaric, benzoic, glycolic, gluconic, succinic, p-toluenesulphonic acids, and arylsulphonic, for example.

The pharmaceutically acceptable excipients described herein, for example, vehicles, adjuvants, carriers or diluents, are well-known to those who are skilled in the art and are readily available to the public. It is preferred that the pharmaceutically acceptable carrier be one that is chemically inert to the active compounds and one that has no detrimental side effects or toxicity under the conditions of use.

The following formulations for oral, aerosol, parenteral, subcutaneous, intravenous, intramuscular, interperitoneal, rectal, and vaginal administration are merely exemplary and are in no way limiting.

Overall, the requirements for effective pharmaceutical carriers for parenteral compositions are well known to those of ordinary skill in the art. See Pharmaceutics and Pharmacy Practice, J.B. Lippincott Company, Philadelphia, Pa., Banker and Chalmers, eds., pages 238-250, (1982), and ASHP Handbook on Injectable Drugs, Toissel, 4th ed., pages 622-630 (1986). Topical formulations, including those that are useful for transdermal drug release, are well-known to those of skill in the art and are suitable in the context of the present invention for application to skin.

Formulations suitable for oral administration require extra considerations considering the nature of the compounds and the possible breakdown thereof if such compounds are administered orally without protecting them from the digestive secretions of the gastrointestinal tract. Such a formulation can consist of (a) liquid solutions, such as an effective amount of the compound dissolved in diluents, such as water, saline, or orange juice; (b) capsules, sachets, tablets, lozenges, and troches, each containing a predetermined amount of the active ingredient, as solids or granules; (c) powders; (d) suspensions in an appropriate liquid; and (e) suitable emulsions. Liquid formulations may include diluents, such as water and alcohols, for example, ethanol, benzyl alcohol, and the polyethylene alcohols, either with or without the addition of a pharmaceutically acceptable surfactant, suspending agent, or emulsifying agent. Capsule forms can be of the ordinary hard- or soft-shelled gelatin type containing, for example, surfactants, lubricants, and inert fillers, such as lactose, sucrose, calcium phosphate, and corn starch. Tablet forms can include one or more of lactose, sucrose, mannitol, corn starch, potato starch, alginic acid, microcrystalline cellulose, acacia, gelatin, guar gum, colloidal silicon dioxide, croscarmellose sodium, talc, magnesium stearate, calcium stearate, zinc stearate, stearic acid, and other excipients, colorants, diluents, buffering agents, disintegrating agents, moistening agents, preservatives, flavoring agents, and pharmacologically compatible excipients. Lozenge forms can comprise the active ingredient in a flavor, usually sucrose and acacia or tragacanth, as well as pastilles comprising the active ingredient in an inert base, such as gelatin and glycerin, or sucrose and acacia, emulsions, gels, and the like containing, in addition to the active ingredient, such excipients as are known in the art.

The compounds of the present invention, alone or in combination with other suitable components, can be made into aerosol formulations to be administered via inhalation. For aerosol administration, the compounds are preferably supplied in finely divided form along with a surfactant and propellant. Typical percentages of compounds are 0.01%-20% by weight, preferably 1%-10%. The surfactant must, of course, be nontoxic, and preferably soluble in the propellant. Representative of such agents are the esters or partial esters of fatty acids containing from 6 to 22 carbon atoms, such as caproic, octanoic, lauric, palmitic, stearic, linoleic, linolenic, olesteric and oleic acids with an aliphatic polyhydric alcohol or its cyclic anhydride. Mixed esters, such as mixed or natural glycerides may be employed. The surfactant may constitute 0.1%-20% by weight of the compounds, preferably 0.25-5%. The balance of the compounds is ordinarily propellant. A carrier can also be included as desired, e.g., lecithin for intranasal delivery. These aerosol formulations can be placed into acceptable pressurized propellants, such as dichlorodifluoromethane, propane, nitrogen, and the like. They also may be formulated as pharmaceuticals for non-pressured preparations, such as in a nebulizer or an atomizer. Such spray formulations may be used to spray mucosa.

It will be understood that, apart from daily doses, the compounds can be administered by other schedules. For instance, the present invention also contemplates depot injection, in which a long acting form of the active compound is injected into the body, such as the muscles. From there the active compound slowly enters the rest of the body, so one injection can last from 1 to 4 weeks or even multiple months. Other form of dosage administrations relate to “once-a-week” pills, in which the ingredient is slowly released over a period of a week, and slow-release patches, e.g. a CDS

(Continuous Delivery System), or Once-a-Day Transdermal Patches.

The aspects and embodiments of the invention are further supported by the following non-limiting examples.

### EXAMPLES

### Example 1

A human female subject (PT1) was treated with pipamperone at a daily dose of 20 mg/day (6 mg in the morning and 14 mg in the evening) for 31 days. During the last three weeks of this period, the subject was treated with transcranial anodal direct current stimulation (tDCS) in daily sessions of 30 minutes in the morning every day of the week except Saturday and Sunday. The tDCS dose was 2 mA. The anode was placed over the left dorsolateral prefrontal cortex and the cathode was placed over the right dorsolateral prefrontal cortex.

The subject was 54 years old, had a high school diploma, and had a medical history of recurrent major depressive episodes since 23 years, and had been treated with continuous intake of an effective dose of antidepressants since 15 years. Although the subject had been suffering from recurrent major depressive episodes for more than 25 years, she was for more than 3 years in full remission under the continuous intake of an effective dose of the antidepressant venlafaxine (150 mg/d). The subject had developed a severe insomnia accompanied with major cognitive deficits such as problems with recalling of memories and word finding difficulties. While keeping a normal level of mood this could not be clinically qualified as part an acute relapse of major depression. While the level of sleep disturbance got more and more severe, subject developed also a relative agitated behaviour during the night with active movements in bed while being awake after sleep onset and trying to solve crosswords for hours.

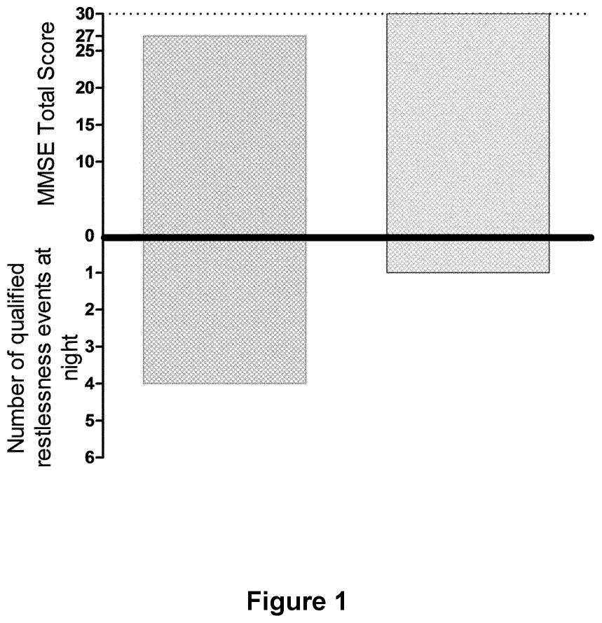

Further clinical examination revealed a significant diminishment of the subject's cognitive performance with a borderline total score on the Mini Mental State Examination (original edition) of 27 (see FIG. 1, left bar). More in dept sleep analysis with the application of a full PSG battery existing of continuous measurement of EEG and EMG combined with a continuous infra-red video- and sound-monitoring during a night stay at our clinical research centre, revealed the occurrence of 4 clinical relevant events of restlessness (see FIG. 1, left bar).

While during sleep the subject had no own noise making behaviour with an observed stable background noise level of +/−40 dB in the room, during these events the subject produced clear-cut enhancement of noise in the room with acute values of more than 50 dB combined with observable muscle activity (as observed by the EMG pattern), a state of being awake (as observed by the EEG) and an agitated behaviour (as observed by the clinical inspection infra-red video images). Surprisingly, these events were very limited in time in terms of several minutes.

To address these clinical phenomena, the subject was initiated on pipamperone 20 mg/day in combination with 15 sessions of tDCS at 2 mA for 30 minutes per session. This combined treatment was continued for a following 3 weeks.

After this acute treatment, we surprisingly observed improvement of the insomnia as well as a full recovery of the subject's cognitive performance as demonstrated with a maximum score of 30 on the MMSE (see FIG. 1, right bar). A re-evaluation of the specific ‘restlessness PSG set-up’ as explained, revealed a significant reduction of the clinical restlessness events a previously detected with 75%, i.e. from 4 to 1 (see FIG. 1, right bar).

During the following 2 months the tDCS treatment was reduced to 2 sessions per week after which tDCS was terminated while the intake of pipamperone 20 mg/d was continued.

Surprisingly, after another 2 months, the subject's cognitive functioning maintained to be fully recovered with a total score on the MMSE of 30/30 revealing a protective effect of a continued intake of a low dose of pipamperone, being a high selective dopamine D4/serotonine 5-HT2A receptor antagonist, on the development of a cognitive deterioration in subjects, such as subjects being at risk to develop a cognitive disorder, for instance due to their combined medical history of major depression and/or the (acute) development of an insomnia accompanied with one or more clinical relevant restlessness events as defined by movement artefacts such as during wake after sleep onset.

### Example 2

A human subject at risk of developing Alzheimer's disease characterised by

(i) an MMSE score of 27;
(ii) insomnia and at least one clinical relevant restlessness event during sleep;
(iii) a medical history of major depressive disorder; and
(iv) a wake after sleep onset (WASO) score of more than 50 minutes
is treated daily with 20 mg pipamperone.

After one month, an increase in cognitive function is evaluated by MMSE.

### Example 3

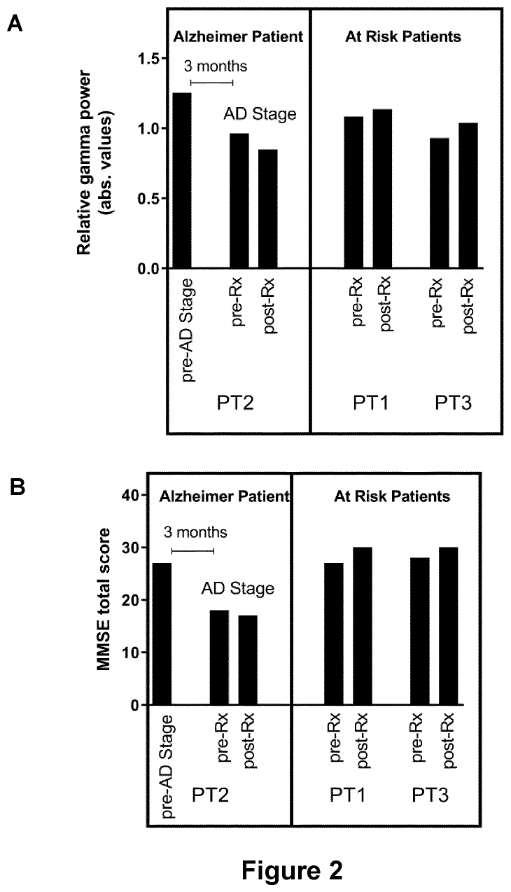

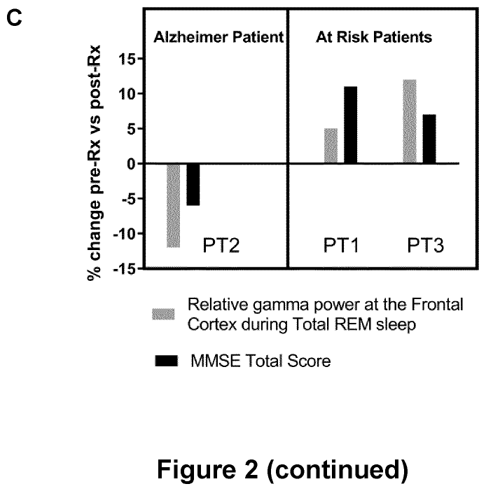

To examine further the surprising finding that subject's cognitive functioning maintained to be fully recovered with a total score on the MMSE of 30/30 (see also FIGS. 2B and 2C) revealing a protective effect of a continued intake of a low dose of pipamperone, being a high selective dopamine D4/serotonine 5-HT2A receptor antagonist, on the development of a cognitive deterioration in subjects, more in depth analysis of the polysomnographic data from this subject (PT1 of Example 1) before and after acute treatment was done by assessing the magnitude of all brain oscillations versus their frequency, the so called ‘EEG spectrum-analysis’.

The recorded signal was decomposed into functionally distinct frequency bands, such as delta (0.5-4 Hz), theta (4-8 Hz), alpha (8-12 Hz), beta (12-30 Hz), and gamma (30-100 Hz), which is one of the most widely used methods.

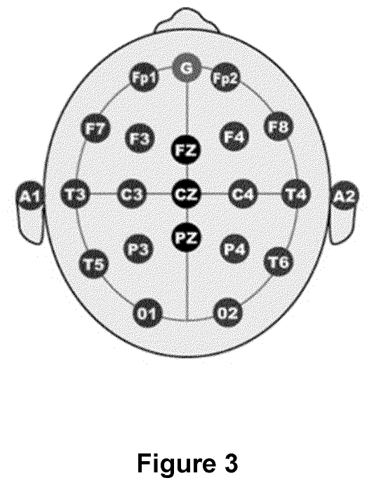

Thereby surprisingly it was found that in accordance with the increase of the cognitive performance—as assessed by the golden standard rating scale the MMSE-, during the REM sleep phase—which is a unique phase of sleep distinguishable by rapid movement of the eyes, accompanied with low muscle tone throughout the body—at the frontal cortex (positions F3, A2, F4, A1; see FIG. 3) the power of the gamma waves over the total power—expressed as the ‘relative gamma power at the frontal cortex’—increased substantially and uniquely since no other changes in brain wave production was found (FIGS. 2A and 2C).

So this present finding indicates that by acting on frontal gamma oscillations, the continuous intake of a high selective Dopamine 4/Selective 5-HT2A receptor antagonist, such as low dose pipamperone, induces a sustained cognitive enhancing effect—as surprisingly found in this subject—by influencing REM sleep-associated cognitive processes on an ongoing basis.

### Example 4

To confirm these findings we applied a similar intervention as described in Example 1 and 3 (PT1) in two other subjects with a medical history of major depressive disorder and current insomnia, whereby in one subject (PT2) a severe cognitive deterioration (as demonstrated by a diminishment of the MMSE total score over the last 3 months prior to starting the pipamperone treatment with 9 points i.e. from 27/30 to 18/30) was observed and whereby in the other subject (PT3) the cognitive difficulties (as expressed by a MMSE of 28/30) were limited.

In contrast with PT1, in PT2 no cognitive improvement could be found (with a MMSE of 17/30 after 6 weeks of treatment, FIGS. 2B and 2C). In addition, a significant decrease of the relative gamma power at the frontal cortex was observed (FIGS. 2A and 2C) and which has been associated with memory decline and as such development of Alzheimer Disease which also took place in this subject as confirmed by further clinical follow-up.

In contrast, in PT3 the same association between improvement/restore of the cognitive performance (as demonstrated by a MMSE of 30/30; FIGS. 2B and 2C) and significant increase of the relative gamma power at the frontal cortex was observed (FIGS. 2A and 2C) as for PT1. Also in this patient the restored cognitive performance sustained under continuous (6 months) intake of low dose pipamperone.

As such, surprisingly we could demonstrate the unique sustained protective effect of a continued intake of a low dose of pipamperone, being a high selective dopamine D4/serotonine 5-HT2A receptor antagonist, on the risk to develop a cognitive deterioration process in subjects, such as subjects being at risk to develop a cognitive disorder, for instance due to their combined medical history of major depression and/or the (acute) development of an insomnia (PT1 and PT3), since in the subject (PT2) with already a clinical relevant cognitive deterioration process in place, no cognitive improving effect with the continuous intake of low dose pipamperone could be detected.

### Example 5

A human subject at risk of developing neurocognitive disorder characterised by

(i) subjective cognitive decline; and
(ii) major depressive disorder or major depressive disorder in remission
is treated daily with 15 mg pipamperone in combination with an effective amount of an antidepressant.

After three months, cognitive function is compared with cognitive function of

(i) a subject treated with 15 mg pipamperone but not at risk of developing neurocognitive disorder; and
(ii) a subject at risk of developing neurocognitive disorder as above but not treated with pipamperone.

### Example 6

A randomized, open-label, rater-blinded, dose-controlled, study is set up in which evaluated flexible dosed low dose pipamperone drops 14 to 20 mg or 4 mg low dose pipamperone drops in elderly participants with major depressive disorder in remission who are experiencing a subjective cognitive decline (SCD) under continuing an adequate antidepressant treatment regimen. Results are compared to similar subjects not treated with pipamperone.

The proportion of participants achieving a clinical meaningful improvement of their cognitive performance as assessed by the Mini Mental State Examination Total Score (MMSE TS) at the Week 8 visit is evaluated.

The primary endpoint is a clinical meaningful improvement of the cognitive performance at the Week 8 visit defined as an improvement of the Mini Mental State Examination (MMSE) total score of up to the maximum of 30/30.

The proportion of participants who achieved a clinical meaningful improvement of their cognitive performance as assessed by the Mini Mental State Examination Total Score (MMSE TS) at the Week 8 visit through the Week 32 visit is evaluated.

The secondary endpoint is an improvement of the Mini Mental State Examination (MMSE) total score of up to the maximum of 30/30 at Week 8 visit through the consecutive 24 weeks until the end of the prospective observation period at Week 32 visit.

Overall Design

This is a randomized, open-label, rater-blinded, dose controlled study to evaluate flexibly dosed low dose pipamperone in the dose range of 14 up to 20 mg per day or low dose pipamperone at the dose of 4 mg per day, both administered under continuing an adequate antidepressant treatment regimen, in participants 60 to 84 years of age, inclusive, with MDD in remission and who are experiencing a Subjective Cognitive Decline (SCD). Results are compared with similar subjects not treated with pipamperone.

The decision for a patient to participate in the study should be determined by a psychiatrist.

The study has 4 phases: an up-to-14-day screening phase, an 8-week acute phase, a 24-week maintenance phase, and a 2-week safety follow-up phase. During the acute phase, participants in the have once-weekly visits from Week 1 to Week 4 and twice-weekly visits from Week 5 to Week 8; during the maintenance phase from Week 9 to Week 32, visits are once monthly.

All participants have a safety follow-up visit 2 weeks following the last dose of study intervention. Participants who discontinue the study intervention early (i.e. discontinue either component of the randomized combination therapy) remain in the study and continue to return for all follow-up study visits through Week 32, according to the Schedule of Activities. The total duration of the study is approximately 36 weeks for all participants. The end of study is considered as the last visit for the last participant in the study.

A total of 40 participants is randomly assigned on Day 1 (baseline) in a 1:1 ratio to 1 of 2 open-label study interventions. The randomization is balanced by using randomly permuted blocks and will be stratified by total number of antidepressant treatments (1; 2 or more [inclusive of current antidepressant treatment at screening used to determine eligibility]).

14-20 mg/day Low Dose Pipamperone Arm: Participants continue to take their current antidepressants in combination with low dose pipamperone 14 mg per day. On Day 1, participants have their first intake at night. During the following visits up to week 32, dosing may be increased at any visit, may remain the same, or may be reduced as determined by the investigator based on efficacy and tolerability. The lowest and highest dose is 14 and 20 mg per day respectively.

If participants cannot tolerate at least 14 mg/d Low Dose Pipamperone by the end of Week 2 (or at any subsequent time during the study), they must have Low Dose Pipamperone discontinued.

4 mg/day Low Dose Pipamperone Arm: Participants continue to take their current antidepressants in combination with low dose pipamperone 4 mg per day. On Day 1, participants have their first intake at night. During the following visits up to week 32, dosing must remain the same.

If participants cannot tolerate 4 mg/d Low Dose Pipamperone by the end of Week 2 (or at any subsequent time during the study), they must have Low Dose Pipamperone discontinued.

Continuing antidepressants: The investigator may optimize the dose of the continuing antidepressants, up to the maximum tolerated dose as per the respective SmPC (or local equivalent, if applicable). Once optimized, a stable dose is maintained; however, dose modifications may be made, if necessary, at the investigator's discretion. The continuing antidepressant being taken by a participant must be labeled for treatment of depression/MDD and the dosage being taken should be according to the respective SmPC (or local equivalent, if applicable).

The study intervention is not considered discontinued if one or more continuing antidepressants are stopped.

Continuing Low Dose Pipamperone:

At the Week 4 visit, therapeutic benefit of the study intervention is evaluated clinically by the investigator. In the evaluation, the full scope of clinical improvement is evaluated including improvement in subjective cognitive decline, general functioning, social functioning, and self-care.

Treatment Continuation Assessment of Study Intervention: Beginning at the Week 8 visit, all participants undergo regular assessments of symptom changes from baseline (i.e. treatment continuation assessment). This is operationalized using the CGI-C clinician-rated scale (referring to study baseline [Day 1]) every 4 weeks.

Study Setting and Patient population:

Inclusion Criteria:

Each potential participant must satisfy all of the following criteria to be enrolled in the study:

1. Male or female, 60 years to 84 years of age, inclusive, at the time of signing the ICF.

2. Participant must meet DSM-5 diagnostic criteria for major depressive disorder (MDD) in full remission, based upon clinical assessment and confirmed by the Mini International Neuropsychiatric Interview (MINI) and the attending psychiatrist. Full remission is defined as no significant MDD symptoms being present directly following the most recent depressed episode for at least 2 months directly following the most recent depressed episode.

3. At screening and baseline, each participant must have an IDS-C30 total score of <34.

4. During screening it must be documented that the participant has had a response within the last moderate to severe depressive episode to at least 1 treatment with an antidepressant taken at an adequate dosage (based on anti-depressive dosages from SmPC [or local equivalent, if applicable]) for an adequate duration of at least 6 weeks.

5. Clinical improvement of a participant on their AD treatment will be retrospectively evaluated in a qualified psychiatric interview performed by an experienced clinician.

At baseline (Day 1) prior to randomization, the investigator will evaluate any changes in the participants signs/symptoms of depression since the screening assessment and confirm that the inclusion criteria for are still met (ie, sustained full remission).

6. Subject must be medically stable based on physical examination, medical history, vital signs (including blood pressure) at screening. If there are any abnormalities that are not specified in the inclusion and exclusion criteria, their clinical significance must be determined by the investigator and recorded in the participant's source documents.

7. Subject must sign an ICF indicating that he or she understands the purpose of, and procedures required for, the study and is willing to participate in the study.

8. Subject is willing and able to adhere to the lifestyle restrictions specified in this protocol.

Exclusion Criteria:

Any potential participant who meets any of the following criteria is excluded from participating in the study:

1. Received ECT, vagal nerve stimulation or deep brain stimulation in any episode of depression.

2. Has a current or prior DSM-5 diagnosis of a psychotic disorder or MDD with psychotic features, bipolar or related disorders (confirmed by the MINI), intellectual disability, autism spectrum disorder, borderline personality disorder, or antisocial personality disorder.

3. Has homicidal ideation or intent, per the investigator's clinical judgment; or has suicidal ideation with some intent to act within 1 month prior to screening, per the investigator's clinical judgment; or based on the C-SSRS, corresponding to a response of “Yes” on Item 4 (active suicidal ideation with some intent to act, without specific plan) or Item 5 (active suicidal ideation with specific plan and intent) for suicidal ideation, or a history of suicidal behavior within the past year prior to screening. Participants reporting suicidal ideation with intent to act or suicidal behavior prior to the start of the acute phase should also be excluded.

4. Has a history (lifetime) of moderate or severe substance use disorder or severe alcohol use disorder according to DSM-5 criteria, except nicotine or caffeine

5. Has a diagnosed neurodegenerative disorder with clinical evidence of cognitive impairment (e.g., Alzheimer's disease, vascular dementia, Parkinson's disease with clinical evidence of cognitive impairment) or any evidence of mild cognitive impairment.

6. Is currently suffering from seizures, has a history of epilepsy, Neuroleptic Malignant Syndrome, or Tardive Dyskinesia.

7. Has one of the following cardiovascular-related conditions:
Cerebrovascular disease with a history of stroke.
History of intracerebral haemorrhage
Uncontrolled brady- or tachyarrhythmias that lead to hemodynamic instability

8. Has clinically significant or unstable respiratory conditions, including, but not limited to:
Uncontrolled pulmonary insufficiency, including chronic obstructive pulmonary disease
Uncontrolled sleep apnea

9. Uncontrolled hypertension despite diet, exercise, or antihypertensive therapy on Day 1 or any history of hypertensive crisis or ongoing evidence of uncontrolled hypertension defined as a supine systolic blood pressure >140 mmHg or diastolic blood pressure >90 mmHg.

Note: On Day 1 (prior to initiation of study intervention) a supine systolic blood pressure >140 mmHg or diastolic blood pressure >90 mmHg is exclusionary.

Participants may have their current antihypertensive medication(s) adjusted during the screening phase and be re-evaluated to assess their blood pressure control prior to randomization.

10. History of additional risk factors for torsade des pointes (e.g. heart failure, hypokalemia, or family history of long QT syndrome).

11. History of, or symptoms and signs suggestive of, liver cirrhosis (e.g. esophageal varices, ascites, and increased prothrombin time) or alanine aminotransferase (ALT) or aspartate aminotransferase (AST) values 3×the upper limit of normal in routine laboratory test or medical record or at screening.

12. Has a fasting triglyceride concentration ≥500 mg/dL at screening.

13. Has positive urine test result(s) for drugs of abuse (including barbiturates, methadone, opiates, cocaine, PCP, and amphetamine/methamphetamine) on Day 1 prior to randomization.

14. Prior intermittent use of cannabinoids prior to the start of the screening phase is not exclusionary as long as the participant does not meet the criteria for substance use disorder. However, a positive urine test result for cannabinoids on Day 1 (baseline) prior to randomization is exclusionary.

15. Has a history (lifetime) of diabetes mellitus, as evidenced by HbA1c >9% in the medical records, routine laboratory test, or history in the 3 months prior to baseline; or diabetic ketoacidosis, hyperglycemic coma, or severe hypoglycemia with loss of consciousness evaluated from medical history.

16. Has untreated glaucoma, current penetrating or perforating eye injury, brain injury, hypertensive encephalopathy, intrathecal therapy with ventricular shunts, or any other condition associated with increased intracranial pressure or increased intraocular pressure or planned eye surgery evaluated from medical history.

17. Has any anatomical or medical condition that, per the investigator's clinical judgment based on assessment, may impede intake or absorption of pipamperone drops.

18. Has a history of malignancy that, in the opinion of the investigator, is not considered cured with minimal risk of recurrence.

19. Has known allergies, hypersensitivity, intolerance, or contraindications to pipamperone and/or any excipients.

20. Is taking any benzodiazepine or has taken any prohibited therapies that would not permit dosing on Day 1 as defined in protocol.

Example 7: Randomized, double-blind, placebo-controlled 2/3-way crossover study to evaluate the effects on cognitive decline in participants with major depressive disorder of repeated administration of oral low dose pipamperone 14 mg, a high selective dopamine 4 and serotonin 2a antagonist, as an add-on medication to an antidepressant

I. Protocol

Depression is a major cause of morbidity and mortality, with global estimates of 300 million treated and untreated individuals worldwide. A depressive state with classical symptoms such as low (depressive/sad) mood, markedly diminished interest in activities, significant weight loss/gain, insomnia or hypersomnia, psychomotor agitation/retardation, excessive fatigue, inappropriate guilt, diminished concentration, and recurrent thoughts of death, persisting for more than 2 weeks is classified as major depressive disorder (MDD).

After a successful treatment with antidepressants known in the art such as SSRI's and SNRI's, many patient diagnosed with a MDD experiences incapacitating residual symptoms such as concentration difficulties related to a higher risk to develop in later stage of life dementia.

Pipamperone acts as an antagonist of the 5-HT2A, 5-HT2B, 5-HT2C, D2, D3, D4, α1-adrenergic and α2-adrenergic receptors. It shows much higher affinity for the 5-HT2A and D4 receptors over the D2 receptor (15-fold in the case of the D4 receptor, and even higher in the case of the 5-HT2A receptor), being regarded as “highly selective” for the former two sites at low doses up to 15 mg per day. ‘Low dose pipamperone’ has low and likely insignificant affinity for the H1 and mACh receptors, as well as for other serotonin and dopamine receptors, but as the molecule is chemically defined as a butyrophenone, it is known in the art as a typical antipsychotic which are related with a negative impact on subjective cognitive performance.

This placebo controlled clinical study was designed to test on a short term i.e. 14 days the effect of low dose pipamperone 14 mg on the subjective cognitive performance in patients diagnosed with a MDD and with a continuous intake of an antidepressant.

Objectives and Endpoints

| | |
|---|---|
| Objectives | Endpoints |

Primary

| | |
|---|---|
| The primary objective of this study is to evaluate | Superior cognitive functioning of the |
| the subjective cognitive performance of | participants defined as a higher weekly |
| participants who have MDD with a current mild | average of the calculated PROMIS T-score |
| or better depressive episode and receiving and | using the PROMIS cognitive function |
| tolerating well any SSRIs or SNRIs for | questionnaire during LOW DOSE |
| depressive symptoms during dosing with LOW | PIPAMPERONE 14 MG AD NOCTUM |
| DOSE PIPAMPERONE 14 MG AD NOCTUM | dosing. |
| compared with placebo dosing. | Note: the questionnaires are completed in |
| | the morning, midday and evening of each |
| | day during the week (21 times/week) |

Key Secondary

| | |
|---|---|
| To assess the sleep quality of the patients of | Superior sleep assessment of the participants |
| participants who have MDD with a current mild | defined as a higher weekly average of the |
| or better depressive episode and receiving and | sleep quality questionnaire during LOW |
| tolerating well any SSRIs or SNRIs for | DOSE PIPAMPERONE 14 MG AD |
| depressive symptoms during dosing with LOW | NOCTUM dosing. |
| DOSE PIPAMPERONE 14 MG AD NOCTUM | Note: the questionnaires are completed in |
| compared with placebo dosing. | the morning of each day during the week (7 |
| | times/week) |

Other Secondary

| | |
|---|---|
| To assess the effect of LOW DOSE | Less sleep-related impairments of the |
| PIPAMPERONE 14 MG AD NOCTUM dosing | participants defined as a higher weekly |
| compared with placebo on following aspects: | average of the calculated PROMIS T- |
| Sleep-related impairments | score using the PROMIS sleep-related |
| Sleepiness during the day by using the | impairments questionnaire during |
| Karolinska Sleepiness Scale | LOW DOSE PIPAMPERONE 14 MG |
| Mood in the morning | AD NOCTUM dosing.^{a)} |
| Mood during the day | Less sleepy during LOW DOSE |
| | PIPAMPERONE 14 MG AD NOCTUM |
| | dosing defined as a lower weekly |
| | average of the Karolinska Sleepiness |
| | Scale.^{a)} |
| | Better mood, more excited and |
| | relaxed, less worried and fearful during |
| | LOW DOSE PIPAMPERONE 14 MG |
| | AD NOCTUM dosing defined as a |
| | lower weekly average of the mood |
| | questionnaire.^{b)} |
| | Note: the questionnaires are completed in: |
| | ^{a)}The morning, midday and evening of |
| | each day during the week (21 times/ |
| | week) |
| | ^{b)}The morning and evening of each |
| | day during the week (14 times/ |
| | week) |

Overall Design

A randomized, double-blind, placebo-controlled 2/3-way crossover study to evaluate the effects of repeated administration of a daily low dose pipamperone 14 mg as an add-on medication in participants with major depressive disorder compared with placebo, both in combination with a continuing SSRI/SNRI, in participants 18 to 74 years of age, inclusive.

Eligible participants were admitted to the study center on Day 1 of Period 1 for first dosing. They were randomly assigned to 1 of the 3 treatment sequences (1 treatment regimen in each of the 3 periods, as shown in Table 1 at the start of the study trial. The sequences were chosen so there will always be two consecutive week of dosing of low dose pipamperone 14 mg.

TABLE 1
Treatment sequence

| Sequence | Period 1 | Period 2 | Period 3 | Period 4 |
|---|---|---|---|---|
| 1 | Treatment A | Treatment A | Treatment B | Treatment B |
| 2 | Treatment B | Treatment A | Treatment A | Treatment B |
| 3 | Treatment B | Treatment B | Treatment A | Treatment A |

All participants randomized to receive Treatment A were administered 14 mg pipamperone before bedtime (i.e. ad noctum). Participants randomized to receive Treatment B were administered placebo containing drinkable water with cassis flavor. Each study week the participants received 10 mL of their study medication in an amber glass bottle with dropper containing 200 droplets. Considering 1 droplet contains 2 mg of pipamperone, the participants were administered 7 droplets of the study medication or placebo bottle in a glass of Peach juice to mask the typical bitter taste of the current formulation of pipamperone.

To ensure the compliance of the participants to the cross-over study design, on day one of period 2, 3, and 4, participants returned to the study center to receive a new glass bottle with dropper, defined by their randomization, and also a new file of questionnaires which they completed during the week. The participants completed these questionnaires during each timepoint of the day as indicated. Participants were considered to have completed the treatment phase of the study if they had administered the medication each day before bedtime and completed all questionnaires during the 4 periods.

Study Population and Number of Participants

Participants met DSM-5 diagnostic criteria for major depressive disorder (MDD) with current mild or better depressive symptoms without psychotic features based upon clinical assessment (DSM-5 296.20, 296.21, 296.25, 296.26, 296.30, 296.31, 296.35 or 296.36) and confirmed by the MINI and the attending psychiatrist.

All participants were receiving and tolerating well any one of the following SSRIs or SNRIs for depressive symptoms in any formulation: citalopram, duloxetine, escitalopram, fluoxetine, paroxetine, sertraline, venlafaxine for at least 12 weeks prior to screening, and expected to continue to take the same drug and dose for the duration of the study.

Participants having any significant primary sleep disorder or receiving medication that causes sedation from 1 week prior to the first dose of study treatment were excluded from participation in this study.

A total of 6 participants were randomly enrolled on Day 1 (baseline) in one of the 3 sequences. The randomization was performed using www.sealedenvelope.com.

Effect Evaluations

The effect of pipamperone 14 mg as add on medication for participants meeting DSM-5 diagnostic criteria for major depressive disorder (MDD) with current mild or better depressive symptoms was assessed by qualitative evaluation of the weekly average scored on the different questionnaires.

The cognitive function was measured using the PROMIS cognitive function questionnaire, completed in the morning, midday and evening. The weekly average of PROMIS T-score of each timepoint was used to assess a cognitive function effect during pipamperone 14 mg dosing compared with placebo. A higher T-score reflects a better cognitive functioning of the participant. This means a participant is a responder when a higher T-score appears during pipamperone 14 mg dosing. Positive responders were further evaluated for the key secondary objective.

The sleep quality of the participants was measured using a scale going from very bad (1) to excellent (5). This questionnaire was only completed in the morning. A weekly average of these results was used to evaluate the sleep quality of the participant. A positive responder, a participant with higher weekly average during pipamperone 14 mg dosing, was further evaluated for the following secondary objectives.

The sleep-related impairments were assessed using the PROMIS Sleep-related impairments questionnaire, completed in the morning, midday and evening. The weekly average of PROMIS T-score of each timepoint was used to assess sleep-related impairments during pipamperone 14 mg dosing compared with placebo. A higher T-score reflects more sleep-related impairments during the day. This means a participant is a positive responder when a lower T-score appears during pipamperone 14 mg dosing.

The sleepiness of the participants was also be measured using the Karolinska Sleepiness Scale going from very sleepy, having great difficulty staying awake, fighting sleep (9) to extremely alert (1). This questionnaire was completed in the morning, midday and evening. A weekly average of these results was used to evaluate the sleepiness of the participant during the day. A positive responder is a participant with a lower weekly average during pipamperone 14 mg dosing compared with placebo.

The mood of the participants was also be measured using a questionnaire which evaluated going from very happy, relaxed, excited, not worried and fearful at all (1) to very sad, not relaxed and excited at all, extremely worried and fearful (5). This questionnaire was completed in the morning and in the evening. A weekly average of these results was used to evaluate the participants mood in the morning and during the day. A positive responder is a participant with a higher weekly average during pipamperone 14 mg dosing compared with placebo.

II. Clinical Study Report

Sceening Results—Baseline Measurments

A total of 6 participants were included. In this section, the participants socio-biographic and medical history information (Table 2) and baseline measurements in relation to their baseline depressive state (as expressed by the total score on the Montgommery-Asberg Depression Rating Scale (MADRS)) and cognitive state (as expressed on the score on the item of ‘concentration difficulties’ of the MADRS—Item 6) (Table 3) is described.

TABLE 2
General information of the participants

| | WL4 | LY1 | EY0 | UU0 | BN6 | XR0 |
|---|---|---|---|---|---|---|
| Male/Female | F | M | F | F | M | M |
| Year of Birth | 1964 | 1965 | 1985 | 1960 | 1963 | 1962 |
| Ongoing | SNRI | SSRI | SNRI | SNRI | SSRI | SNRI |
| Antidepressant | | | | | | |
| Education | More | More | More | More | College | Some |
| level | than | than | than | than | or | secondary |
| | high | high | high | high | Baccalaureate | or high |
| | school | school | school | school | | school |
| | | | | | | education |

History of depression

| | | | | | | |
|---|---|---|---|---|---|---|
| 1^{st} episode | Mid | 2006 | 2015 | 1995 | 2015 | 1981 |
| | 2015 | | | | | |
| 2^{nd} episode | Mid | 2020 | January | 2003 | March | March |
| | 2017 | | 2019 | | 2020 | 2020 |
| 3^{rd} episode | End | NA | September | 2009 | NA | NA |
| | 2018 | | 2019 | | | |
| 4^{th} episode | April | NA | NA | 2019 | NA | NA |
| | 2020 | | | | | |
| Current | No | No | No | Yes | Partial | No |
| remission? | | | | | | |
| Comorbidity | None | Obstructive | None | Hyper- | None | None |
| | | sleep disorder, | | thyroidic | | |
| | | in remission | | | | |
| | | Asthma | | | | |

TABLE 3
Baseline measurements

| | WL4 | LY1 | EY0 | UU0 | BN6 | XR0 |
|---|---|---|---|---|---|---|
| MADRAS | 32 | 1 | 18 | 0 | 0 | 11 |
| Total score | | | | | | |
| MADRS | 4 | 0 | 1 | 0 | 0 | 3 |
| Cognitive item | | | | | | |

Primary Endpoint—Cognitive Function

The two participants with a clinical relevant high score on the MADRS item 6 (concentration difficulties) (XR0 and WL4) had the highest improvement of their cognitive functioning during pipamperone 14 mg dosing compared with placebo.

Key Secondary Endpoint—Sleep Quality

All participants were reporting better quality of sleep better during pipamperone 14 mg dosing compared with placebo which is in line with in the art known sleep improving effect of pipamperone.

Additional Secondary Endpoints
1. Sleep-related impairments: no clear difference between the two treatment paradigms were detected.
2. Karolinska Sleepiness Scale: no clear difference between the two treatment paradigms were detected.
3. Mood: no clear difference between the two treatment paradigms were detected.

Statistical Testing & Results

Since the in this trial used qualitative outcome measurement responder vs. non-responder for both treatment paradigms pipamperone 14 mg versus Placebo, statistical testing of the primary end point using the Fisher exact test was executed.

Surprisingly, taken in to account that low dose pipamperone 14 mg showed in all participants a known in the art and thus expected sleep improving effect but without any improvement during the day in sleep related impairments, sleepiness and mood status, an unknown clinical relevant and statistical significant (p<0.05) improvement of the subjective cognitive function versus placebo was detected.

SUBJECTIVE COGNITIVE DECLINE

| | | NON | Marginal |
|---|---|---|---|
| | RESPONDER | RESPONDER | Row Totals |
| LOW DOSE | 50% | 17% | 67% |
| PIPAMPERONE 14 MG | | | |
| AD NOCTUM | | | |
| Placebo | 33% | 0% | 33% |
| Marginal Column Totals | 83% | 17% | 100 |
| | | | (Grand Total) |

The Fisher exact test statistic value is 0.0006.

The result is significant at p<0.05.

Since the observed subjective cognitive improving effect of pipamperone 14 mg was not related to the impact of better sleep on other sleep related daily functioning such as sleepiness and mood and since this cognitive enhancing effect was most relevant in these patients with the most severe status of subjective cognitive decline at baseline (MADRS item 6 score), a not in the art known direct subjective cognitive enhancing effect of low dose pipamperone 14 mg in patients with a residual major depressive state was found.

### Example 8

An international, double-blind, centrally randomized (stratified), multi-centre study in more than 400 patients suffering from moderate to severe MDD in up to 40 sites in the USA was conducted. Eligible out-patients were treated once daily (QD) with a fixed dose of either pipamperone 15 mg/citalopram 20 mg (Week 1)—pipamperone 15 mg/citalopram 40 mg (Week 2-10); citalopram 20 mg (Week 1)—citalopram 40 mg (Week 2-10); or pipamperone 15 mg alone (Week 1-10) in a 1:1:1 ratio in a double-blind fashion for 10 weeks. Study visits were conducted 1, 2, 3, 4, 6, 8 and 10 weeks after study treatment initiation. Possible withdrawal effects were assessed 1 week after study treatment withdrawal.

A blood sample for pharmacokinetic analysis was collected when drawing blood for routine biochemistry. Patients who provided written informed consent to participate to the study were asked to provide their consent to participate also to the non-mandatory pharmacogenetic study. Patient related outcomes were collected electronically (ePRO) at study visits prior to visiting the investigator by using an Interactive Voice Response System (IVRS) via telephone. Patients wishing or choosing to discontinue the study treatment prematurely were encouraged to continue to provide their scores, safety data and medications taken, up to the scheduled study end, by telephone.

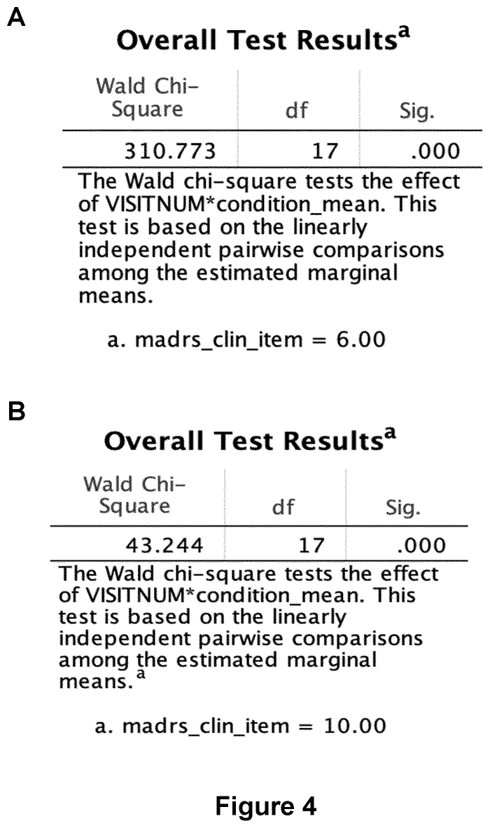

After 2, 3, 4, 6, and 10 weeks, patients were evaluated. The results are provided in FIGS. 4 to 7.

FIG. 4 demonstrates a statistically significant enhancement in cognitive function after 10 weeks, as well as a significant reduction in suicidal thoughts after 10 weeks in subjects treated with pipamperone compared to subjects treated with citalopram.

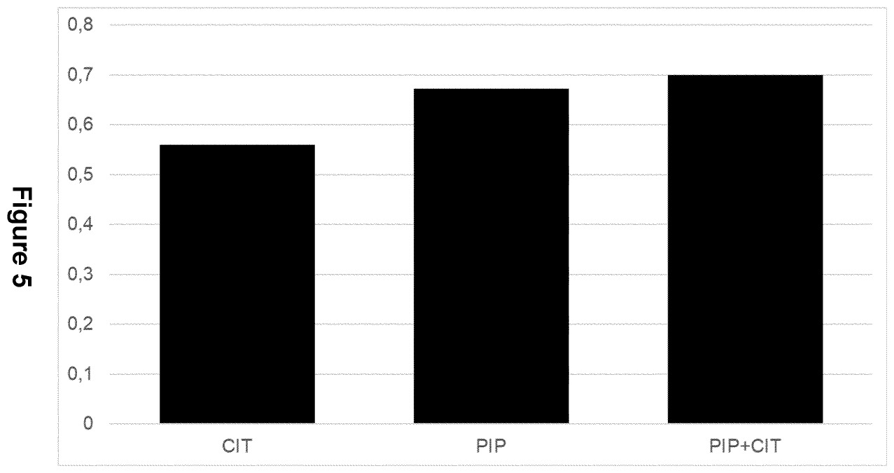

FIG. 5 demonstrates the superior cognitive enhancement rate (determined as remission of reduced ability to read/communicate evaluated according to MADRS item 6) of pipamperone treatment (and combined pipamperone/citalopram treatment) compared to citalopram treatment after 10 weeks of treatment.

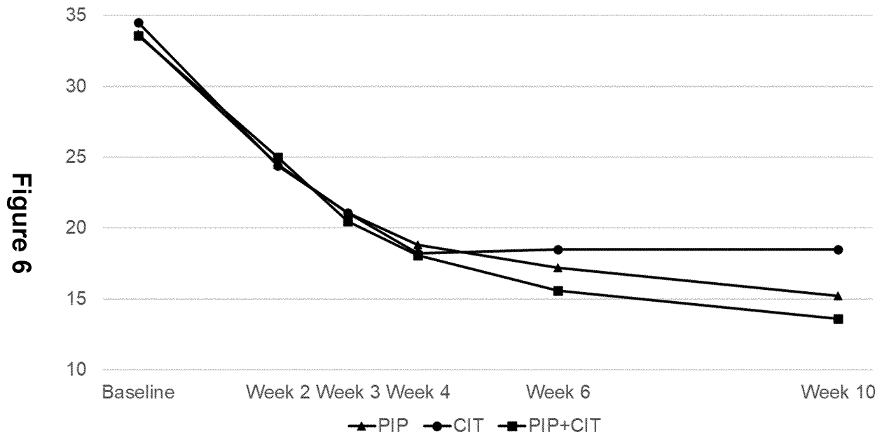

FIG. 6 demonstrates a superior decrease of severity of major depressive disorder, in particular after 6 weeks, even more so after 10 weeks, after treatment with pipamperone (and pipamperone combined with citalopram) compared to treatment with citalopram, as evaluated by the MADRS total score.

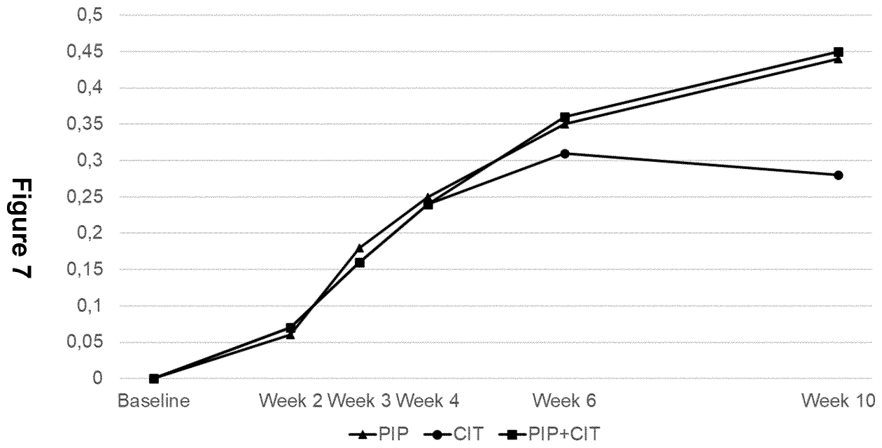

FIG. 7 demonstrates a superior increase in the proportion of patients in remission of major depressive disorder, in particular after 6 weeks, even more so after 10 weeks, after treatment with pipamperone (and pipamperone combined with citalopram) compared to treatment with citalopram, as evaluated by the MADRS total score.

1. A D4 and preferably also 5-HT2A receptor antagonist, reverse agonist, or partial agonist for use in preventing, delaying or postponing the onset or progression, and/or treating of a cognitive disorder in a subject, preferably for delaying or postponing the onset of a cognitive disorder, and/or for use in maintaining or improving cognitive function in a subject, and/or for maintaining or increasing (relative) gamma power in a subject, preferably at the (frontal) cortex and/or during (REM) sleep.

2. A D4 and preferably also 5-HT2A receptor antagonist, reverse agonist, or partial agonist for use in increasing the MMSE score, and/or decreasing the number of qualified clinical restlessness events during sleep in a subject, and/or for maintaining or increasing (relative) gamma power in a subject, preferably at the (frontal) cortex and/or during (REM) sleep.

3. The D4 and preferably also 5-HT2A receptor antagonist, reverse agonist, or partial agonist for use according to any of claims 1 to 2 which is pipamperone.

4. The D4 and preferably also 5-HT2A receptor antagonist, reverse agonist, or partial agonist for use according to claim 3, wherein said pipamperone is to be administered at a daily dose ranging from 5 to 20 mg or 4 to 20 mg, preferably 5 to 20 mg.

5. The D4 and preferably also 5-HT2A receptor antagonist, reverse agonist, or partial agonist for use according to any of claims 1 to 4, wherein said cognitive disorder is (dementia) caused by Alzheimer's Disease, substance-related persisting dementia, vascular dementia, dementia due to HIV disease, dementia with Lewy bodies, dementia due to head trauma/chronic traumatic encephalopathy, dementia due to Parkinson Disease, dementia due to Huntington Disease, dementia due to Pick Disease (frontotemporal dementia), dementia due to prions (e.g. Creutzfedt-Jacob Disease), Normal Pressure Hydrocephalus, subdural hematoma, posterior cortical atrophy, corticobasal degeneration, progressive supranuclear palsy, mixed dementia, medication side effects, chronic alcoholism, tumors or infections in the brain, toxins (e.g. lead), Wernicke-Korsakoff, hypothyroidism, vitamin B12 deficiency, Lyme disease, and neurosyphillis, Behcet's disease, multiple sclerosis, sarcoidosis, Sjögren's syndrome, systemic lupus erythematosus, celiac disease, and non-celiac gluten sensitivity, vitamin B12 deficiency, folate deficiency, niacin deficiency, and infective causes including cryptococcal meningitis, AIDS, Lyme disease, progressive multifocal leukoencephalopathy, subacute sclerosing panencephalitis, syphilis, and Whipple's disease, Alexander disease, Canavan disease, Cerebrotendinous xanthomatosis, Dentatorubral-pallidoluysian atrophy, Epilepsy, Fatal familial insomnia, Fragile X-associated tremor/ataxia syndrome, Glutaric aciduria type 1, Krabbe's disease, Maple syrup urine disease, Niemann-Pick disease type C, Neuronal ceroid lipofuscinosis, Neuroacanthocytosis, Organic acidemias, Pelizaeus-Merzbacher disease, Sanfilippo syndrome type B, Spinocerebellar ataxia type 2, Urea cycle disorders, amnestic disorders due to a general medical condition, substance-induced persisting amnestic disorder, mild cognitive impairment disorder, preferably (dementia) caused by Alzheimer's Disease.

6. The D4 and preferably also 5-HT2A receptor antagonist, reverse agonist, or partial agonist for use according to any of claims 1 to 5, wherein said subject has an increased risk of developing said cognitive disorder.

7. The D4 and preferably also 5-HT2A receptor antagonist, reverse agonist, or partial agonist for use according to any of claims 1 to 6, wherein said subject has subjective cognitive decline (SCD).

8. The D4 and preferably also 5-HT2A receptor antagonist, reverse agonist, or partial agonist for use according to any of claims 1 to 7, wherein said subject has pre-mild cognitive impairment (pre-MCI) or mild cognitive impairment (MCI).

9. The D4 and preferably also 5-HT2A receptor antagonist, reverse agonist, or partial agonist for use according to any of claims 1 to 8, wherein said subject has a pre-prodromal neurodegenerative disease or a prodromal neurodegenerative disease, preferably wherein said neurodegenerative disease in Alzheimer's disease.

10. The D4 and preferably also 5-HT2A receptor antagonist, reverse agonist, or partial agonist for use according to any of claims 1 to 8, wherein said subject has an pre-clinical or asymptomatic neurodegenerative disease, preferably wherein said neurodegenerative disease in Alzheimer's disease.

11. The D4 and preferably also 5-HT2A receptor antagonist, reverse agonist, or partial agonist for use according to any of claims 1 to 10, wherein said subject has an MMSE score ranging from 25 to 29, preferably 26 to 29, more preferably 27 to 29 or ranging from 25 to 30, preferably 26 to 30, more preferably 27 to 30.

12. The D4 and preferably also 5-HT2A receptor antagonist, reverse agonist, or partial agonist for use according to any of claims 1 to 11, wherein said subject has a sleep disorder, preferably (acute) insomnia.

13. The D4 and preferably also 5-HT2A receptor antagonist, reverse agonist, or partial agonist for use according to any of claims 1 to 12, wherein said subject exhibits at least one clinical relevant restlessness event during sleep.

14. The D4 and preferably also 5-HT2A receptor antagonist, reverse agonist, or partial agonist for use according to claim 13, wherein said clinical relevant restlessness event is determined by polysomnography (PSG).

15. The D4 and preferably also 5-HT2A receptor antagonist, reverse agonist, or partial agonist for use according to claim 13 or 14, wherein said clinical relevant restlessness event is or includes movement artefact(s).

16. The D4 and preferably also 5-HT2A receptor antagonist, reverse agonist, or partial agonist for use according to any of claims 1 to 15, wherein said clinical restlessness event during sleep is characterized by one or more, preferably all, of (i) noise-making (>50 db), (ii) an EMG signal, (iii) an EEG alpha signal which precedes, is part of or follows the period of movement artefact, or (iv) visible movement.

17. The D4 and preferably also 5-HT2A receptor antagonist, reverse agonist, or partial agonist for use according to any of claims 1 to 16, wherein said subject has (a medical history of) an affective disorder.

18. The D4 and preferably also 5-HT2A receptor antagonist, reverse agonist, or partial agonist for use according to claim 17, wherein said affective disorder is (mild) major depressive disorder, bipolar disorder, or anxiety disorder, preferably (mild) major depressive disorder.

19. The D4 and preferably also 5-HT2A receptor antagonist, reverse agonist, or partial agonist for use according to any of claims 1 to 18, wherein said subject has a wake after sleep onset (WASO) score of at least 30 minutes, preferably at least 40 minutes, more preferably at least 50 minutes.

20. The D4 and preferably also 5-HT2A receptor antagonist, reverse agonist, or partial agonist for use according to any of claims 1 to 19, wherein said subject has:
(i) an MMSE score ranging from 25 to 29, preferably 26 to 29, more preferably 27 to 29, or ranging from 25 to 30, preferably 26 to 30, more preferably 27 to 30;
(ii) a sleep disorder, preferably (acute) insomnia, and/or exhibits at least one clinical relevant restlessness event during sleep;
(iii) a medical history of an affective disorder, preferably (mild) major depressive disorder; and
(iv) a wake after sleep onset (WASO) score of at least 30 minutes, preferably at least 40 minutes, more preferably at least 50 minutes.

21. The D4 and preferably also 5-HT2A receptor antagonist, reverse agonist, or partial agonist for use according to any of claims 1 to 20, wherein said subject:
(i) has an affective disorder, preferably (mild) major depressive disorder or major depressive disorder in remission; and
(ii) further has
(a) subjective cognitive decline;
(b) (pre-)mild cognitive disorder;
(c) (pre-)prodomal neurodegenerative disorder, preferably wherein said disorder is Alzheimer's disease; and/or
(d) pre-clinical or asymptomatic neurodegenerative disorder, preferably wherein said disorder is Alzheimer's disease.

22. The D4 and preferably also 5-HT2A receptor antagonist, reverse agonist, or partial agonist for use according to any of claims 1 to 20, wherein said subject has:
(i) (mild) major depressive disorder or major depressive disorder in remission; and
(ii) subjective cognitive decline.
